(19) United States
(12) Patent Application Publication
(10) Pub. No.: US 2026/0351340 A1
(43) Pub. Date: Nov. 5, 2026

(54) METHODS AND SYSTEMS FOR IDENTIFYING HLA-I LOSS OF HETEROZYGOSITY

(71) Applicant: Foundation Medicine, Inc., Boston, MA (US)

(72) Inventor: Meagan Kathleen MONTESION, Somerville, MA (US)

(73) Assignee: Foundation Medicine, Inc., Boston, MA (US)

(21) Appl. No.: 19/099,413

(22) PCT Filed: Jul. 24, 2023
(86) PCT No.: PCT/US2023/070866

§ 371 Date: Jan. 28, 2025

Related U.S. Application Data
(60) Provisional application No. 63/393,487, filed on Jul. 29, 2022.
(60) Provisional application No. 63/397,718, filed on Aug. 12, 2022.

(51) Int. Cl. G16B 20/10; G16B 20/20

(52) U.S. Cl. CPC G16B 20/10; G16B 20/20

(57) ABSTRACT

Methods and systems for determining a loss of heterozygosity (LOH) status of the one or more HLA-T genes are described. The methods may comprise, for example: identifying, using one or more processors, a plurality of genomic segments in a sample, selecting, from the plurality of genomic segments, one or more genomic segments that overlap with one or more HLA-I genes in the sample, and determining whether a tumor purity of the sample is in a predetermined range. In accordance with a determination that the tumor purity of the sample is in the predetermined range, the system can obtain a copy number value of the one or more selected genomic segments. The system can then determine a loss of heterozygosity (LOH) status of the one or more HLA-I genes based on the copy number value.

### CROSS REFERENCE TO RELATED APPLICATIONS

This application claims the benefit of U.S. Provisional Application No. 63/393,487, filed Jul. 29, 2022, and U.S. Provisional Application No. 63/397,718, filed Aug. 12, 2022, the contents of which are hereby incorporated by reference in their entirety.

### FIELD OF THE DISCLOSURE

The present disclosure relates generally to methods and systems for analyzing genomic profiling data, and more specifically to methods and systems for identifying HLA-I loss of heterozygosity (LOH) in a sample.

### BACKGROUND

Loss of heterozygosity (LOH) of the HLA class I (HLA-I) genes, e.g., HLA-A, HLA-B, or HLA-C, can be understood as a mechanism of immune evasion and may be correlated with worse outcomes in patients treated with immune checkpoint inhibitors. Thus, determining the HLA-I LOH status of a sample in instances may be helpful for identifying treatment options for patients. While the HLA-I LOH status may be determined based on allelic imbalance information and copy number modeling information, there are instances where both are not available. For example, certain diagnostic tests may not provide information regarding both allelic imbalance and copy number modeling. Thus, systems and methods for determining the HLA-I LOH status of a sample, particularly when at least one of allelic imbalance or copy number modeling data is unavailable, would be useful to inform patient care and improve patient outcomes.

### BRIEF SUMMARY

Disclosed herein are methods and systems for determining HLA-I loss of heterozygosity (LOH) of a sample. While HLA-I LOH status of tumor samples has been determined using a combination of allelic imbalance and copy number modeling, there may be instances where both allelic imbalance information and copy number modeling information are not available. One or more embodiments of this disclosure provide methods for identifying HLA-I LOH when allelic imbalance information is available, but copy number variation information is unavailable. One or more embodiments of this disclosure provide methods for identifying HLA-I LOH when copy number variation information is available, but allelic imbalance information is unavailable.

Embodiments of the present disclosure are directed to determining a loss of heterozygosity (LOH) status of a sample based on a copy number value. In one or more embodiments, these methods can include: providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, at one or more processors, sequence read data for the plurality of sequence reads; identifying, using one or more processors, a plurality of genomic segments in the sample based on the sequence read data; selecting, using the one or more processors, selecting, using the one or more processors, from the plurality of genomic segments, one or more genomic segments that overlap with one or more HLA-I genes in the sample; determining, using the one or more processors, whether a tumor purity of the sample is in a predetermined range; when the tumor purity of the sample is in the predetermined range, obtaining, using the one or more processors, a copy number value of the one or more selected genomic segments; and determining, using the one or more processors, a loss of heterozygosity (LOH) status of the one or more HLA-I genes based on the copy number value.

In one or more embodiments, the method for determining a LOH status of the one or more HLA-I genes further comprises performing, using the one or more processors, a quality control procedure prior to obtaining the copy number value; and foregoing determining the LOH status of the one or more HLA-I genes in accordance with a determination that the sample is associated with a quality control issue. In such embodiments, wherein the quality control issue includes a sample contamination, a confirmed transplant, a noisy copy number, or a combination thereof.

In one or more embodiments, the sample may be at least one of a tissue biopsy sample or a liquid biopsy sample. In such embodiments, the biopsy sample may comprise a normal tissue and a tumor tissue. In one or more embodiments, the predetermined range comprises a tumor purity between 20-95%. In one or more embodiments, the LOH status may be determined without baiting the one or more HLA-I genes. In one or more embodiments, the one or more HLA-I genes comprise an HLA-A gene, an HLA-B gene, an HLA-C gene, or a combination thereof. In one or more embodiments, the method further comprises determining a zygosity score based on a copy number of the one or more selected genomic segments. In one or more embodiments, a zygosity score of about 1 corresponds to a positive LOH status and a zygosity score of about 2 indicates a non-positive LOH status.

In one or more embodiments, the subject is suspected of having or is determined to have cancer. In one or more embodiments, the cancer is a B cell cancer, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In one or more embodiments, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia. In such embodiments, the method further comprises treating the subject with an anti-cancer therapy.

In one or more embodiments, the anti-cancer therapy comprises a targeted anti-cancer therapy. In such embodiments, the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In one or more embodiments, the method further comprises obtaining the sample from the subject. In one or more embodiments, the sample comprises one or more samples. In one or more embodiments, the sample comprises a tissue biopsy sample, a liquid biopsy sample, or a normal tissue sample. In one or more embodiments, the sample is a liquid biopsy sample and comprises blood, plasma, cerebrospinal fluid, sputum, stool, urine, or saliva. In one or more embodiments, the sample is a liquid biopsy sample and comprises circulating tumor cells (CTCs). In one or more embodiments, the sample is a liquid biopsy sample and comprises cell-free DNA (cfDNA), circulating tumor DNA (ctDNA), or any combination thereof.

In one or more embodiments, determining the copy number value determining, using the one or more processors, a ploidy of the sample, coverage ratio data of the sample, allele fraction data of the sample, segmentation data of the sample, and a copy number model for the one or more HLA-I genes; determining, using the one or more processors, segment copy numbers for the one or more selected genomic segments that overlap with the one or more HLA-I genes in the sample based on at least the coverage ratio data, the allele fraction data, the segmentation data, and the copy number model; detecting, using the one or more processors, a presence of amplifications or deletions for one or more gene loci of the one or more HLA-I genes based on a segment copy number of the segment copy numbers for a corresponding genomic segment of the one or more selected genomic segments; and calling, using the one or more processors, the copy number of the one or more selected genomic segments based on the detected presence of amplifications or deletions for the one or more gene loci of the one or more HLA-I genes.

In one or more embodiments, the plurality of nucleic acid molecules comprises a mixture of tumor nucleic acid molecules and non-tumor nucleic acid molecules. In such embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In one or more embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In one or more embodiments, the one or more adapters comprise amplification primers, flow cell adaptor sequences, substrate adapter sequences, or sample index sequences. In one or more embodiments, the captured nucleic acid molecules are captured from the amplified nucleic acid molecules by hybridization to one or more bait molecules. In such embodiments, the one or more bait molecules comprise one or more nucleic acid molecules, each comprising a region that is complementary to a region of a captured nucleic acid molecule.

In one or more embodiments, amplifying nucleic acid molecules comprises performing a polymerase chain reaction (PCR) amplification technique, a non-PCR amplification technique, or an isothermal amplification technique. In one or more embodiments, the sequencing comprises use of a massively parallel sequencing (MPS) technique, whole genome sequencing (WGS), whole exome sequencing, targeted sequencing, direct sequencing, or Sanger sequencing technique. In such embodiments, the sequencing comprises massively parallel sequencing, and the massively parallel sequencing technique comprises next generation sequencing (NGS). In one or more embodiments, the sequencer comprises a next generation sequencer.

In one or more embodiments, one or more of the plurality of sequencing reads overlap one or more gene loci within one or more subgenomic intervals in the sample. In such embodiments, the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In one or more embodiments, the one or more gene loci comprise ABLI, ACVRIB, AKT1, AKT2, AKT3, ALK, ALOX12B, AMER1, APC, AR, ARAF, ARFRP1, ARIDIA, ASXL1, ATM, ATR, ATRX, AURKA, AURKB, AXIN1, AXL, BAP1, BARDI, BCL2, BCL2L1, BCL2L2, BCL6, BCOR, BCORLI, BCR, BRAF, BRCA1, BRCA2, BRD4, BRIP1, BTG1, BTG2, BTK, CALR, CARD11, CASP8, CBFB, CBL, CCND1, CCND2, CCND3, CCNEI, CD22, CD274, CD70, CD74, CD79A, CD79B, CDC73, CDH1, CDK12, CDK4, CDK6, CDK8, CDKNIA, CDKNIB, CDKN2A, CDKN2B, CDKN2C, CEBPA, CHEK1, CHEK2, CIC, CREBBP, CRKL, CSFIR, CSF3R, CTCF, CTNNAI, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFII, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNFIA, HRAS, HSD3B1, ID3, IDH1, IDH2, IGFIR, IKBKE, IKZF1, INPP4B, IRF2, IRF4, IRS2, JAK1, JAK2, JAK3, JUN, KDMSA, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKB1A, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCDILG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKARIA, PRKCI, PTCH1, PTEN, PTPN11, PTPRO, QKI, RACI, RAD21, RAD51, RAD51B, RAD51C, RADSID, RADS2, RAD54L, RAFI, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSCI, WHSCIL1, WT1, XPO1, XRCC2, ZNF217, ZNF703, or any combination thereof.

In one or more embodiments, the one or more gene loci comprise ABL, ALK, ALL, B4GALNT1, BAFF, BCL2, BRAF, BRCA, BTK, CD19, CD20, CD3, CD30, CD319, CD38, CDS2, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAKI, JAK2, JAK3, KIT, KRAS, MEK, MET, MSI-H, mTOR, PARP, PD-1, PDGFR, PDGFRa, PDGFRB, PD-L1, PI3Kõ, PIGF, PTCH, RAF, RANKL, RET, ROS1, SLAMF7, VEGF, VEGFA, VEGFB, or any combination thereof.

In one or more embodiments, the method further comprises generating, by the one or more processors, a report indicating the LOH status of the one or more HLA-I genes. In such embodiments the method further comprises transmitting the report to a healthcare provider. In one or more embodiments, the report is transmitted via a computer network or a peer-to-peer connection.

Embodiments of the present disclosure comprise systems and methods for determining a loss of heterozygosity (LOH) status of the one or more HLA-I genes based on the copy number value. The methods comprise identifying, using one or more processors, a plurality of genomic segments in a sample; selecting, using the one or more processors, from the plurality of genomic segments, one or more genomic segments that overlap with one or more HLA-I genes in the sample; determining, using the one or more processors, whether a tumor purity of the sample is in a predetermined range; when the tumor purity of the sample is in the predetermined range, obtaining, using the one or more processors, a copy number value of the one or more selected genomic segments; and determining, using the one or more processors, a loss of heterozygosity (LOH) status of the one or more HLA-I genes based on the copy number value.

In one or more embodiments, the method for determining a LOH status of the one or more HLA-I genes further comprises performing, using the one or more processors, a quality control procedure prior to obtaining the copy number value; and foregoing determining the LOH status of the one or more HLA-I genes in accordance with a determination that the sample is associated with a quality control issue. In such embodiments, the quality control issue includes a sample contamination, a confirmed transplant, a noisy copy number, or a combination thereof.

In one or more embodiments, the sample may be at least one of a tissue biopsy sample or a liquid biopsy sample. In such embodiments, the tissue biopsy sample may comprise a normal tissue sample and/or a tumor tissue sample.

In one or more embodiments, the predetermined range comprises a tumor purity between 20-95%. In one or more embodiments, the LOH status may be determined without baiting the one or more HLA-I genes. In one or more embodiments, the one or more HLA-I genes comprise an HLA-A gene, an HLA-B gene, an HLA-C gene, or a combination thereof.

In one or more embodiments, determining the loss of heterozygosity (LOH) status can comprise: determining the LOH status to be indeterminate if the copy number value is at about a first threshold; determining the LOH status to be positive if the copy number value is at about a second threshold; and determining the LOH status to be negative if the copy number value is above a third threshold. In such embodiments, the first threshold can be about 0, the second threshold can be about 1, and the third threshold can be about 2. In one or more embodiments, when the copy number value is at the third threshold, the system can determine, using the one or more processors, whether the LOH status is a neutral LOH; and the system can determine, using the one or more processors, the LOH status to be (1) non-positive (e.g., negative) if the LOH status does not correspond to the neutral LOH, or (2) positive if the LOH status corresponds to the neutral LOH.

In one or more embodiments, determining the copy number comprises: determining, using the one or more processors, a ploidy of the sample, coverage ratio data of the sample, allele fraction data of the sample, segmentation data of the sample, and a copy number model for the one or more HLA-I genes; determining, using the one or more processors, segment copy numbers for the one or more selected genomic segments that overlap with the one or more HLA-I genes in the sample based on at least the coverage ratio data, the allele fraction data, the segmentation data, and the copy number model; detecting, using the one or more processors, a presence of amplifications or deletions for one or more gene loci of the one or more HLA-I genes based on a segment copy number for a corresponding genomic segment of the one or more selected genomic segments; and calling, using the one or more processors, the copy number of the one or more selected genomic segments based on the detected presence of amplifications or deletions for the one or more gene loci of the one or more HLA-I genes. In one or more examples, the copy number of the one or more selected genomic segments matches the sample ploidy and neither an amplification nor a deletion is detected. In such embodiments, the system may determine a negative LOH status or a positive LOH status for a neutral LOH.

In such embodiments, determining the coverage ratio data can comprise: aligning, using the one or more processors, a plurality of sequence reads of the one or more selected genomic segments that overlap with the one or more HLA-I genes in the sample to a reference genome; aligning, using the one or more processors, a plurality of sequence reads of one or more selected genomic segments that overlap with the one or more HLA-I genes in a control sample to the reference genome; determining, using the one or more processors, a number of sequence reads that overlap each of the one or more gene loci of the one or more HLA-I genes within the one or more selected genomic segments in the sample; and determining, using the one or more processors, a number of sequence reads that overlap each of the one or more gene loci of the one or more HLA-I genes within the one or more selected genomic segments in the control sample.

In one or more embodiments, determining the allele fraction data can comprise: aligning, using the one or more processors, a plurality of sequence reads of the one or more selected genomic segments that overlap with the one or more HLA-I genes in the sample in the sample to a reference genome; detecting a number of alleles present at a gene locus of the one or more gene loci of the one or more HLA-I genes; and determining an allele fraction for at least one of the alleles present at the gene locus.

In one or more embodiments, determining the segmentation data can comprise: aligning, using the one or more processors, a plurality of sequence reads of the one or more selected genomic segments that overlap with the one or more HLA-I genes in the sample to a reference genome; obtaining, using the one or more processors, aligned sequence read data based on the aligned plurality of sequence reads of the one or more selected genomic segments in the sample; processing, using the one or more processors, the aligned sequence read data of the sample, the coverage ratio data of the sample, and the allele fraction data of the sample using a pruned exact linear time (PELT) method; and determining a number of partial segments of the one or more selected genomic segments in the sample, wherein each partial segment has a same copy number.

In one or more embodiments, determining the copy number can comprise: determining, using the one or more processors, normalized sequence read data and minor allele frequency (MAF) data based on the one or more selected genomic segments that overlap with the one or more HLA-I genes in the sample; segmenting, using the one or more processors, the normalized sequence read data using whole-genome segmentation; fitting, using the one or more processors, one or more copy number models to the segmented normalized sequence read data and the MAF data; and determining the copy number based on the fitted segmented normalized sequence read data and the MAF data. In such embodiments, the genome segmentation comprises a circular binary segmentation algorithm, an HMM based method, a Wavelett based method, or a Cluster along Chromosomes method. In one or more embodiments, the method for determining the copy number can comprise segmenting, using the one or more processors, a genomic sequence of the one or more selected genomic sequences that overlap with the one or more HLA-I genes in the sample into partial genomic segments of equal copy number.

In one or more embodiments, the method for determining the LOH status of the one or more HLA-I genes can further comprise determining a zygosity score based on the copy number of the one or more selected genomic segments. In such embodiments, a zygosity score of about 1 corresponds to a positive LOH status and a zygosity score of about 2 indicates a non-positive LOH status.

In one or more embodiments, determining the zygosity score comprises: determining, using the one or more processors, a sequence coverage input (SCI), based on a number of reads of a selected genomic segment of the one or more selected genomic segments that overlap the one or more HLA-I genes in the sample; determining, using the one or more processors, a single nucleotide polymorphism (SNP) allele frequency input (SAFI), based on a SNP allele frequency for each of a plurality of selected germline SNPs in the sample; and determining, using the one or more processors, a variant allele frequency input (VAFI), based on an allele frequency for a variant associated with the LOH status; obtaining, using the one or more processors, values based on the SCI and the SAFI for: a genomic segment total copy number (C value), for each of the one or more selected genomic segments in the sample; a genomic segment minor allele copy number (M value), for each of the one or more selected genomic segments in the sample; and sample purity (p); and obtaining, using the one or more processors, the zygosity score as a function of the C value and the M value. In such embodiments, a genomic segment of the one or more selected genomic segments in the sample comprises a plurality of subgenomic intervals, and wherein determining the zygosity score further comprises assigning, using the one or more processors, SCI values to the plurality of subgenomic intervals.

In one or more embodiments, the methods in accordance with embodiments of this disclosure can further comprise determining, using the one or more processors, a treatment decision for a subject based on the LOH status of the one or more HLA-I genes. In such embodiments, if the LOH status of the one or more HLA-I genes is a non-positive LOH status, the treatment comprises an immune checkpoint inhibitor (ICI). In one or more embodiments, if the LOH status of the one or more HLA-I genes is a positive LOH status, the treatment comprises a chemotherapy treatment, a non-ICI targeted treatment, a radiation therapy, a hormone therapy, or a combination thereof.

In one or more embodiments, the methods in accordance with embodiments of this disclosure can further comprise administering, using the one or more processors, a treatment to a subject based on the LOH status of the one or more HLA-I genes. In such embodiments, if the LOH status of the one or more HLA-I genes is a non-positive LOH status, the treatment comprises an immune checkpoint inhibitor (ICI) treatment. In one or more embodiments, if the LOH status of the one or more HLA-I genes is a positive LOH status, the treatment comprises a chemotherapy treatment, a non-ICI targeted treatment, a radiation therapy, a hormone therapy, or a combination thereof.

In one or more embodiments, the methods in accordance with embodiments of this disclosure can further comprise assessing, using the one or more processors, an immunotherapy resistance of a subject based on the LOH status of the one or more HLA-I genes. In one or more embodiments, the methods in accordance with embodiments of this disclosure can further comprise monitoring, using the one or more processors, an immunotherapy resistance of a subject based on the LOH status of the one or more HLA-I genes over time. In one or more embodiments, the methods in accordance with embodiments of this disclosure can further comprise predicting, using the one or more processors, one or more clinical outcomes based on the LOH status of the one or more HLA-I genes. In one or more embodiments, the methods in accordance with embodiments of this disclosure can further comprise predicting, using the one or more processors, an overall survival of the subject based on the LOH status of the one or more HLA-I genes.

Embodiments of the present disclosure comprise methods for diagnosing a disease, the method comprising diagnosing that a subject has the disease based on a determination of a loss of heterozygosity (LOH) status for a sample from the subject, wherein the LOH status is determined according to any of the methods for determining the LOH status described above.

Embodiments of the present disclosure comprise methods for selecting an anti-cancer therapy, the method comprising responsive to determining a loss of heterozygosity (LOH) status for a sample from a subject, selecting an anti-cancer therapy for the subject, wherein the LOH status is determined according to any of the methods for determining the LOH status described above.

Embodiments of the present disclosure comprise methods for treating a cancer in a subject, comprising responsive to determining a loss of heterozygosity (LOH) for a sample from the subject, administering an effective amount of an anti-cancer therapy to the subject, wherein the LOH status is determined according to any of the methods for determining the LOH status described above.

Embodiments of the present disclosure comprise methods for monitoring cancer progression or recurrence in a subject, the method comprising: determining a first loss of heterozygosity (LOH) status in a first sample obtained from the subject at a first time point according to any of the methods for determining the LOH status described above; determining a second LOH status in a second sample obtained from the subject at a second time point; and comparing the first LOH status to the second LOH status, thereby monitoring the cancer progression or recurrence.

In one or more embodiments, any of the methods for determining the LOH status described above further comprise determining, identifying, or applying a value of a loss of heterozygosity (LOH) status for the sample as a diagnostic value associated with the sample. In one or more embodiments, any of the methods for determining the LOH status described can be used to make suggested treatment decisions for the subject.

Embodiments of the present disclosure further comprise a system comprising: one or more processors; and a memory communicatively coupled to the one or more processors and configured to store instructions that, when executed by the one or more processors, cause the system to: identify, using the one or more processors, a plurality of genomic segments in a sample; select, using the one or more processors, from the plurality of genomic segments, one or more genomic segments that overlap with one or more HLA-I genes in the sample; determine, using the one or more processors, whether a tumor purity of the sample is in a predetermined range; when the tumor purity of the sample is in the predetermined range, obtain, using the one or more processors, a copy number value of the one or more selected genomic segments; determine, using the one or more processors, a loss of heterozygosity (LOH) status of the one or more HLA-I genes based on the copy number value.

Embodiments of the present disclosure further comprise a non-transitory computer-readable storage medium storing one or more programs, the one or more programs comprising instructions, which when executed by one or more processors of a system, cause the system to: identify, using the one or more processors, a plurality of genomic segments in a sample; select, using the one or more processors, from the plurality of genomic segments, one or more genomic segments that overlap with one or more HLA-I genes in the sample; determine, using the one or more processors, whether a tumor purity of the sample is in a predetermined range; when the tumor purity of the sample is in the predetermined range, obtain, using the one or more processors, a copy number value of the one or more selected genomic segments; and determine, using the one or more processors, a loss of heterozygosity (LOH) status of the one or more HLA-I genes based on the copy number value.

Embodiments of the present disclosure can further comprise methods comprising: identifying, using one or more processors, a plurality of genomic segments in a sample; selecting, using the one or more processors, from the plurality of genomic segments, one or more genomic segments that overlap with one or more HLA-I genes in the sample without baiting the one or more HLA-I genes; determining, using the one or more processors, whether a tumor purity of the sample is in a predetermined range; when the tumor purity of the sample is in the predetermined range, obtaining, using the one or more processors, a copy number value of the one or more selected genomic segments; determining, using the one or more processors, a loss of heterozygosity (LOH) status of the one or more HLA-I genes.

Embodiments of the present disclosure are directed to determining a loss of heterozygosity (LOH) status based on the gene MAF of the sample. In one or more embodiments, these methods can include: providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, at one or more processors, sequence read data for the plurality of sequence reads; identifying, using one or more processors, a plurality of genomic segments in the sample based on the sequence read data; selecting, using the one or more processors, receiving, at one or more processors, sequence read data for the plurality of sequence reads, wherein one or more reads of the plurality of sequence reads correspond to a plurality of genomic segments in a sample, wherein the one or more sequence reads are aligned with a reference sequence; determining, using the one or more processors: whether a number of sequence reads in the one or more sequence reads reads exceeds a predetermined sequence read threshold; whether the plurality of genomic segments are germline heterozygous at one or more HLA-I gene loci in the sample; and whether a tumor content of the sample is above a predetermined tumor content threshold; when the plurality of genomic segments are germline heterozygous, the number of sequence reads in the one or more sequence reads exceeds the predetermined sequence read threshold, and the tumor content is above the predetermined tumor content threshold: determining, using the one or more processors, a gene minor allele frequency (MAF) of the sample and a segment MAF of the sample; and determining, using the one or more processors, a LOH status based on the gene MAF of the sample, the segment MAF of the sample, and the tumor content of the sample.

In one or more embodiments of the present disclosure, the sample comprises at least one of a tissue biopsy sample, a liquid biopsy sample, or a normal control. In one or more embodiments of the present disclosure, the tumor content corresponds to a maximum somatic allele frequency (MSAF). In one or more embodiments of the present disclosure, the tumor content corresponds to a tumor purity.

In one or more embodiments of the present disclosure, the one or more HLA-I genes comprise an HLA-A gene, an HLA-B gene, an HLA-C gene, or a combination thereof. In one or more embodiments, the method further comprises sequencing the one or more sequence reads corresponding to the plurality of genomic segments in the sample that have been baited for the one or more HLA-I genes. In one or more embodiments, the method further comprises aligning, using the one or more processors, the one or more sequence reads corresponding to a plurality of genomic segments in the sample to the reference sequence, the reference sequence associated with the one or more HLA-I gene loci. In one or more embodiments, the method further comprises obtaining a sample from the subject. In one or more embodiments, determining whether the plurality of genomic segments in the sample are germline heterozygous at the one or more HLA-I genes in the sample comprises determining, using the one or more processors, an allele fraction of the sample at the one or more HLA-I gene loci is about 0.5.

In one or more embodiments, determining whether the one or more sequence reads derived from the sample exceeds the predetermined sequence read threshold comprises: aligning, using the one or more processors, the one or more sequence reads corresponding to the plurality of genomic segments in the sample to the reference sequence, the reference sequence comprising one or more sequences for an HLA allele of the one or more HLA-I genes; determining, using the one or more processors, the number of sequence reads in the one or more sequence reads aligned with the reference sequence at the one or more HLA-I gene loci; and comparing, using the one or more processors, the number of sequence reads to the predetermined sequence read threshold.

In one or more embodiments, the sequence read threshold is one of 1000 reads, 1100 reads, 1200 reads, 1300 reads, 1400 reads, 1500 reads, 1600 reads, 1700 reads, 1800 reads, 1900 reads, 2000 reads, 2100 reads, 2200 reads, 2300 reads, 2400 reads, 2500 reads, 2600 reads, 2700 reads, 2800 reads, 2900 reads, or 3000 reads. The method of any of claims 79 to 88, wherein the tumor content threshold corresponds to a tumor content of 1%, 5%, or 10%.

In one or more embodiments, the determining the second estimate of the tumor content of the sample based on the allele frequency determination comprises: determining whether a quality metric for the plurality of values is greater than a second threshold; based on a determination that the quality metric for the plurality of values is greater than the second threshold, determining the second estimate for the tumor content of the sample based on a first determination of somatic allele frequency, and based on a determination that the quality metric for the plurality of values is less than or equal to the second threshold, determining the second estimate for the tumor content of the sample based on a second determination of somatic allele frequency. In one or more embodiments, the tumor content is a value indicative of a ratio of circulating tumor DNA (ctDNA) to total cell-free DNA (cfDNA) in the sample.

In one or more embodiments, determining the second estimate of the tumor content of the sample based on the allele frequency determination comprises: determining whether a quality metric for the plurality of values is greater than a second threshold; based on a determination that the quality metric for the plurality of values is greater than the second threshold, determining the second estimate for the tumor content of the sample based on a first determination of somatic allele frequency; and based on a determination that the quality metric for the plurality of values is less than or equal to the second threshold, determining the second estimate for the tumor content of the sample based on a second determination of somatic allele frequency.

In one or more embodiments, the subject is suspected of having or is determined to have cancer. In one or more embodiments, the cancer is a B cell cancer, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, bepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar bypereosinophilia, chronic eosinophilic leukemia, neuroendocrine cancers, or a carcinoid tumor.

In one or more embodiments, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia. In such embodiments, the method further comprises treating the subject with an anti-cancer therapy.

In one or more embodiments, the anti-cancer therapy comprises a targeted anti-cancer therapy. In such embodiments, the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fibj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In one or more embodiments, the method further comprises obtaining the sample from the subject. In one or more embodiments, the sample comprises one or more samples. In one or more embodiments, the sample comprises a tissue biopsy sample, a liquid biopsy sample, or a normal tissue sample. In one or more embodiments, the sample is a liquid biopsy sample and comprises blood, plasma, cerebrospinal fluid, sputum, stool, urine, or saliva. In one or more embodiments, the sample is a liquid biopsy sample and comprises circulating tumor cells (CTCs). In one or more embodiments, the sample is a liquid biopsy sample and comprises cell-free DNA (cfDNA), circulating tumor DNA (ctDNA), or any combination thereof.

In one or more embodiments, the plurality of nucleic acid molecules comprises a mixture of tumor nucleic acid molecules and non-tumor nucleic acid molecules. In such embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In one or more embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In one or more embodiments, the one or more adapters comprise amplification primers, flow cell adaptor sequences, substrate adapter sequences, or sample index sequences. In one or more embodiments, the captured nucleic acid molecules are captured from the amplified nucleic acid molecules by hybridization to one or more bait molecules. In such embodiments, the one or more bait molecules comprise one or more nucleic acid molecules, each comprising a region that is complementary to a region of a captured nucleic acid molecule.

In one or more embodiments, amplifying nucleic acid molecules comprises performing a polymerase chain reaction (PCR) amplification technique, a non-PCR amplification technique, or an isothermal amplification technique. In one or more embodiments, the sequencing comprises use of a massively parallel sequencing (MPS) technique, whole genome sequencing (WGS), whole exome sequencing, targeted sequencing, direct sequencing, or Sanger sequencing technique. In such embodiments, the sequencing comprises massively parallel sequencing, and the massively parallel sequencing technique comprises next generation sequencing (NGS). In one or more embodiments, the sequencer comprises a next generation sequencer.

In one or more embodiments, the one or more sequence reads overlap one or more gene loci within one or more subgenomic intervals in the sample. In such embodiments, the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In one or more embodiments, the one or more gene loci comprise ABLI, ACVR1B, AKT1, AKT2, AKT3, ALK, ALOX12B, AMER1, APC, AR, ARAF, ARFRP1, ARIDIA, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCNDI, CCND2, CCND3, CCNE1, CD22, CD274, CD70, CD74, CD79A, CD79B, CDC73, CDH1, CDK12, CDK4, CDK6, CDK8, CDKNIA, CDKNIB, CDKN2A, CDKN2B, CDKN2C, CEBPA, CHEK1, CHEK2, CIC, CREBBP, CRKL, CSFIR, CSF3R, CTCF, CTNNAI, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFII, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNFIA, HRAS, HSD3B1, ID3, IDH1, IDH2, IGFIR, IKBKE, IKZF1, INPP4B, IRF2, IRF4, IRS2, JAK1, JAK2, JAK3, JUN, KDMSA, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPKI, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKB1A, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCDILG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKARIA, PRKCI, PTCH1, PTEN, PTPN11, PTPRO, QKI, RACI, RAD21, RAD51, RAD51B, RAD51C, RADSID, RAD52, RAD54L, RAFI, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCIL1, WT1, XPO1, XRCC2, ZNF217, ZNF703, or any combination thereof.

In one or more embodiments, the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.

In one or more embodiments, the method further comprises generating, by the one or more processors, a report indicating the LOH status of the one or more HLA-I genes in the sample. In such embodiments the method further comprises transmitting the report to a healthcare provider. In one or more embodiments, the report is transmitted via a computer network or a peer-to-peer connection.

Embodiments of the present disclosure can further comprise methods comprising: receiving, at the one or more processors, one or more sequence reads corresponding to a plurality of genomic segments in a sample, wherein the one or more sequence reads are aligned with a reference sequence; determining, using the one or more processors: whether a number of one or more sequence reads exceeds a predetermined sequence read threshold; whether the plurality of genomic segments are germline heterozygous at one or more HLA-I genes in the sample; and whether a tumor content of the sample is above a predetermined tumor content threshold; when the plurality of genomic segments are germline heterozygous, the number of sequence reads in the one or more sequence reads exceeds the predetermined read threshold, and the tumor content is above the predetermined tumor content threshold: determining, using the one or more processors, a gene minor allele frequency (MAF) of the sample and a segment MAF of the sample; and determining, using the one or more processors, a loss of heterozygosity (LOH) status based on the gene MAF of the sample, the segment MAF of the sample, and the tumor content of the sample.

In one or more embodiments of the present disclosure, the sample comprises at least one of a tissue biopsy sample, a liquid biopsy sample, or a normal control. In one or more embodiments, the sample comprises one or more samples. In one or more embodiments of the present disclosure, the tumor content corresponds to a maximum somatic allele frequency (MSAF). In one or more embodiments of the present disclosure, the tumor content corresponds to a tumor purity. In one or more embodiments of the present disclosure, the one or more HLA-I genes comprise an HLA-A gene, an HLA-B gene, an HLA-C gene, or a combination thereof.

In one or more embodiments of the present disclosure the methods can further comprise sequencing, using the one or more processors, the one or more reads corresponding to the plurality of genomic segments in the sample that have been baited for the one or more HLA-I genes.

In one or more embodiments of the present disclosure the methods can further comprise aligning, using the one or more processors, the one or more reads corresponding to a plurality of genomic segments in the sample to the reference sequence.

In one or more embodiments of the present disclosure determining whether the plurality of genomic segments in the sample are germline heterozygous at the one or more HLA-I gene loci in the sample comprises determining, using the one or more processors, an allele fraction of the sample at the one or more HLA-I gene gene loci is about 0.5.

In one or more embodiments of the present disclosure, determining whether the one or more sequence reads derived from the sample exceeds the predetermined sequence read threshold comprises: aligning, using the one or more processors, the one or more sequence reads corresponding to the plurality of genomic segments in the sample to the reference sequence, the reference sequence comprising one or more sequences for an HLA allele of the one or more HLA-I genes; and determining, using the one or more processors, the number of sequence reads aligned with the reference sequence; and comparing, using the one or more processors, the number of sequence reads to the predetermined sequence read threshold.

In one or more embodiments of the present disclosure, the sequence read threshold is one of 1000 reads, 1100 reads, 1200 reads, 1300 reads, 1400 reads, 1500 reads, 1600 reads, 1700 reads, 1800 reads, 1900 reads, 2000 reads, 2100 reads, 2200 reads, 2300 reads, 2400 reads, 2500 reads, 2600 reads, 2700 reads, 2800 reads, 2900 reads, or 3000 reads.

In one or more embodiments of the present disclosure, the tumor content threshold corresponds to a tumor content of 1%, 5%, or 10%.

In one or more embodiments of the present disclosure, determining whether the tumor content of the sample is above a predetermined tumor content threshold comprises: receiving, at the one or more processors, a plurality of values, each value indicative of an allele fraction at a gene locus within the plurality of genomic segments in the sample; determining, by the one or more processors, a certainty metric value indicative of a dispersion of the plurality of values; determining, by the one or more processors, a first estimate of the tumor content of the sample, the first estimate based on the certainty metric value for the sample and a predetermined relationship between one or more stored certainty metric values and one or more stored tumor fraction values; determining, by the one or more processors, whether a value associated with the first estimate is greater than a first threshold; based on a determination that the value associated with the first estimate is greater than the first threshold, outputting, by the one or more processors, the first estimate as the tumor content of the sample; and based on a determination that the value associated with the first estimate is less than or equal to the first threshold: determining, by the one or more processors, a second estimate of the tumor content of the sample; outputting, by the one or more processors, the second estimate as the tumor content of the sample; and comparing the tumor content of the sample to the tumor content threshold.

In one or more embodiments of the present disclosure, the tumor content is a value indicative of a ratio of circulating tumor DNA (ctDNA) to total cell-free DNA (cfDNA) in the sample.

In one or more embodiments of the present disclosure, determining the second estimate of the tumor content of the sample based on the allele frequency determination comprises: determining whether a quality metric for the plurality of values is greater than a second threshold; based on a determination that the quality metric for the plurality of values is greater than the second threshold, determining the second estimate for the tumor content of the sample based on a first determination of somatic allele frequency, and based on a determination that the quality metric for the plurality of values is less than or equal to the second threshold, determining the second estimate for the tumor content of the sample based on a second determination of somatic allele frequency.

In one or more embodiments methods in accordance with the present disclosure, can further comprise training a model for determining the LOH status of a sample, the training comprising: receiving, at the one or more processors, one or more reads corresponding to one or more selected genomic segments in a paired sample, wherein the one or more reads corresponding to the one or more selected genomic segments are aligned with the reference sequence; for each paired sample: fitting, using the one or more processors, one or more values associated with the one or more reads corresponding to the one or more selected genomic segments to a model; determining, using the one or more processors, a LOH status of the corresponding paired sample based on the fitted model; and determining, using the one or more processors, the predetermined sequence read threshold and the tumor content threshold based on the fitted model and the LOH status for the one or more selected genomic segments in the paired sample.

In one or more embodiments, the methods in accordance with embodiments of this disclosure can further comprise determining, using the one or more processors, a treatment decision for a subject based on the LOH status of the one or more HLA-I genes. In such embodiments, if the LOH status of the one or more HLA-I genes is a non-positive LOH status, the treatment comprises an immune checkpoint inhibitor (ICI). In one or more embodiments, if the LOH status of the one or more HLA-I genes is a positive LOH status, the treatment comprises a chemotherapy treatment, a non-ICI targeted treatment, a radiation therapy, a hormone therapy, or a combination thereof.

In one or more embodiments, the methods in accordance with embodiments of this disclosure can further comprise administering, using the one or more processors, a treatment to a subject based on the LOH status of the one or more HLA-I genes. In such embodiments, if the LOH status of the one or more HLA-I genes is a non-positive LOH status, the treatment comprises an immune checkpoint inhibitor (ICI) treatment. In one or more embodiments, if the LOH status of the one or more HLA-I genes is a positive LOH status, the treatment comprises a chemotherapy treatment, a non-ICI targeted treatment, a hormone therapy, a radiation therapy, or a combination thereof.

In one or more embodiments, the methods in accordance with embodiments of this disclosure can further comprise assessing, using the one or more processors, an immunotherapy resistance of a subject based on the LOH status of the one or more HLA-I genes. In one or more embodiments, the methods in accordance with embodiments of this disclosure can further comprise monitoring, using the one or more processors, an immunotherapy resistance of a subject based on the LOH status of the one or more HLA-I genes over time. In one or more embodiments, the methods in accordance with embodiments of this disclosure can further comprise predicting, using the one or more processors, one or more clinical outcomes based on the LOH status of the one or more HLA-I genes. In one or more embodiments, the methods in accordance with embodiments of this disclosure can further comprise predicting, using the one or more processors, an overall survival of the subject based on the LOH status of the one or more HLA-I genes.

Embodiments of the present disclosure comprise methods for diagnosing a disease, the method comprising diagnosing that a subject has the disease based on a determination of a loss of heterozygosity (LOH) status for a sample from the subject, wherein the LOH status is determined according to any of the methods for determining the LOH status described above.

Embodiments of the present disclosure comprise methods for selecting an anti-cancer therapy, the method comprising responsive to determining a loss of heterozygosity (LOH) status for a sample from a subject, selecting an anti-cancer therapy for the subject, wherein the LOH status is determined according to any of the methods for determining the LOH status described above.

Embodiments of the present disclosure comprise methods for treating a cancer in a subject, comprising responsive to determining a loss of heterozygosity (LOH) for a sample from the subject, administering an effective amount of an anti-cancer therapy to the subject, wherein the LOH status is determined according to any of the methods for determining the LOH status described above.

Embodiments of the present disclosure comprise methods for monitoring cancer progression or recurrence in a subject, the method comprising: determining a first loss of heterozygosity (LOH) status in a first sample obtained from the subject at a first time point according to any of the methods for determining the LOH status described above; determining a second LOH status in a second sample obtained from the subject at a second time point; and comparing the first LOH status to the second LOH status, thereby monitoring the cancer progression or recurrence.

In one or more embodiments, any of the methods for determining the LOH status described above further comprise determining, identifying, or applying a value of a loss of heterozygosity (LOH) status for the sample as a diagnostic value associated with the sample. In one or more embodiments, any of the methods for determining the LOH status described can be used to make suggested treatment decisions for the subject.

Embodiments of the present disclosure further comprise systems for determining the HLA-I LOH status of a sample, the system comprising: one or more processors; and a memory communicatively coupled to the one or more processors and configured to store instructions that, when executed by the one or more processors, cause the system to: receive, at the one or more processors, one or more sequence reads corresponding to a plurality of genomic segments in a sample, wherein the one or more sequence reads are aligned with a reference sequence; determine, using the one or more processors: whether a number of sequence reads in the one or more reads exceeds a predetermined sequence read threshold; whether the plurality of genomic segments are germline heterozygous at one or more HLA-I gene loci in the sample; and whether a tumor content of the sample is above a predetermined tumor content threshold; when the plurality of genomic segments are germline heterozygous, the number of sequence reads in the one or more sequence reads exceeds the predetermined sequence read threshold, and the tumor content is above the predetermined tumor content threshold: determine, using the one or more processors, a gene minor allele frequency (MAF) of the sample and a segment MAF of the sample; and determine, using the one or more processors, a loss of heterozygosity (LOH) status of the sample based on the gene MAF of the sample, the segment MAF of the sample, and the tumor content of the sample.

Embodiments of the present disclosure further comprise non-transitory computer-readable storage mediums storing one or more programs, the one or more programs comprising instructions, which when executed by one or more processors of a system, cause the system to: receive, at the one or more processors, one or more sequence reads corresponding to a plurality of genomic segments in a sample, wherein the one or more sequence reads are aligned with a reference sequence; determine, using the one or more processors: whether a number of sequence reads in the one or more sequence reads exceeds a predetermined read threshold; whether the plurality of genomic segments are germline heterozygous at one or more HLA-I gene loci in the sample; and whether a tumor content of the sample is above a predetermined tumor content threshold; when the plurality of genomic segments are germline heterozygous, the number of sequence reads in the one or more reads exceeds the predetermined sequence read threshold, and the tumor content is above the predetermined tumor content threshold: determine, using the one or more processors, a gene minor allele frequency (MAF) of the sample and a segment MAF of the sample; and determine, using the one or more processors, a loss of heterozygosity (LOH) status of the sample based on the gene MAF of the sample, the segment MAF of the sample, and the tumor content of the sample.

Embodiments of the present disclosure provide methods for treating a subject having a cancer, comprising determining a loss of heterozygosity (LOH) status of HLA-I gene in a sample from the subject. In one or more embodiments, the method comprises: identifying a plurality of genomic segments in a sample; selecting from the plurality of genomic segments, one or more genomic segments that overlap with one or more HLA-I genes in the sample; determining whether a tumor purity of the sample is in a predetermined range; when the tumor purity of the sample is in the predetermined range, obtaining a copy number value of the one or more selected genomic segments; determining a loss of heterozygosity (LOH) status of the one or more HLA-I genes based on the copy number value, wherein an HLA-I LOH positive status is determined if the copy number value is associated with a first threshold; and treating the subject with a non-immune-oncology (IO) therapy if the subject is determined to have the HLA-I LOH positive status.

Embodiments of the present disclosure provide methods for selecting a treatment for a subject having a cancer, comprising determining a loss of heterozygosity (LOH) status of HLA-I gene in a sample from the subject. In one or more embodiments, the method comprises: identifying a plurality of genomic segments in a sample; selecting from the plurality of genomic segments, one or more genomic segments that overlap with one or more HLA-I genes in the sample; determining whether a tumor purity of the sample is in a predetermined range; when the tumor purity of the sample is in the predetermined range, obtaining a copy number value of the one or more selected genomic segments; determining a loss of heterozygosity (LOH) status of the one or more HLA-I genes based on the copy number value, wherein an HLA-I LOH positive status is determined if the copy number value is associated with a first threshold; and identifying the subject for treatment with a non-immune-oncology (IO) therapy if the subject is determined to have the HLA-I LOH positive status.

Embodiments of the present disclosure provide methods for identifying a subject having a cancer for treatment with an immune oncology (IO) therapy, the method comprising determining a loss of heterozygosity (LOH) status of HLA-I gene in a sample from the subject. In one or more embodiments, the method comprises: identifying a plurality of genomic segments in a sample; selecting from the plurality of genomic segments, one or more genomic segments that overlap with one or more HLA-I genes in the sample; determining whether a tumor purity of the sample is in a predetermined range; when the tumor purity of the sample is in the predetermined range, obtaining a copy number value of the one or more selected genomic segments; determining a loss of heterozygosity (LOH) status of the one or more HLA-I genes based on the copy number value, wherein an HLA-I LOH positive status is determined if the copy number value is associated with a first threshold; and identifying the subject for treatment with a non-immune-oncology (IO) therapy if the subject is determined to have the HLA-I LOH positive status.

Embodiments of the present disclosure provide methods for identifying one or more treatment options for a subject having a cancer, the method comprising: identifying a plurality of genomic segments in a sample; selecting from the plurality of genomic segments, one or more genomic segments that overlap with one or more HLA-I genes in the sample; determining whether a tumor purity of the sample is in a predetermined range; when the tumor purity of the sample is in the predetermined range, obtaining a copy number value of the one or more selected genomic segments; determining a loss of heterozygosity (LOH) status of the one or more HLA-I genes based on the copy number value; and generating a report comprising one or more treatment options identified for the subject based at least in part on the HLA-I LOH status determined for the sample, wherein: the subject is identified as one who may benefit from treatment with an immuno-oncology (IO) therapy if the subject is determined to have an HLA-I LOH non-positive status, and the subject is identified as one who may benefit from treatment with a non-IO therapy if the subject is determined to have the HLA-I LOH positive status.

Embodiments of the present disclosure provide methods for stratifying a subject having cancer for treatment with an immuno-oncology (IO) therapy, the method comprising: identifying a plurality of genomic segments in a sample; selecting from the plurality of genomic segments, one or more genomic segments that overlap with one or more HLA-I genes in the sample; determining whether a tumor purity of the sample is in a predetermined range; when the tumor purity of the sample is in the predetermined range, obtaining a copy number value of the one or more selected genomic segments; determining a loss of heterozygosity (LOH) status of the one or more HLA-I genes based on the copy number value, wherein an HLA-I LOH positive status is determined if the copy number value is associated with a first threshold; and treating the subject with a non-IO therapy if the subject is determined to have the HLA-I LOH positive status.

Embodiments of the present disclosure provide methods for predicting survival of a subject having cancer, the method comprising: identifying a plurality of genomic segments in a sample; selecting from the plurality of genomic segments, one or more genomic segments that overlap with one or more HLA-I genes in the sample; determining whether a tumor purity of the sample is in a predetermined range; when the tumor purity of the sample is in the predetermined range, obtaining a copy number value of the one or more selected genomic segments; determining a loss of heterozygosity (LOH) status of the one or more HLA-I genes based on the copy number value, wherein an HLA-I LOH positive status is determined if the copy number value is associated with a first threshold and wherein: if the subject is determined to have the HLA-I LOH positive status, the subject is predicted to have shorter survival when treated with an IO therapy, as compared to a subject that was determined to have an HLA-I LOH non-positive status.

Embodiments of the present disclosure provide methods for predicting survival of a subject having cancer, the method comprising: identifying a plurality of genomic segments in a sample; selecting from the plurality of genomic segments, one or more genomic segments that overlap with one or more HLA-I genes in the sample; determining whether a tumor purity of the sample is in a predetermined range; when the tumor purity of the sample is in the predetermined range, obtaining a copy number value of the one or more selected genomic segments; and determining a loss of heterozygosity (LOH) status of the one or more HLA-I genes based on the copy number value, wherein an HLA-I LOH positive status is determined if the copy number value is associated with a first threshold and wherein if the subject is determined to have the HLA-I LOH positive status, the subject is predicted to have longer survival when treated with a non-immuno-oncology (IO) therapy, as compared to a subject that was treated with an IO therapy.

Embodiments of the present disclosure comprise methods for treating a subject having a cancer, comprising: receiving a plurality of sequence reads corresponding to a plurality of genomic segments in a sample from a subject, wherein the plurality of sequence reads are aligned with a reference sequence; determining: whether a number of sequence reads in the plurality of sequence reads exceeds a predetermined sequence read threshold; whether the plurality of genomic segments are germline heterozygous at one or more HLA-I gene loci in the sample; and whether a tumor content of the sample is above a predetermined tumor content threshold; when the plurality of genomic segments are germline heterozygous, the number of sequence reads in the plurality of sequence reads exceeds the predetermined sequence read threshold, and the tumor content is above the predetermined tumor content threshold: determining a gene minor allele frequency (MAF) of the sample and a segment MAF of the sample; and determining a loss of heterozygosity (LOH) status based on the gene MAF of the sample, the segment MAF of the sample, and the tumor content of the sample, wherein an HLA-I LOH positive status is determined if the tumor content is above the predetermined tumor content threshold and the gene MAF is below a first gene threshold and the segment MAF is below a first segment threshold; treating the subject with a non-immune-oncology (IO) therapy if the subject is determined to have an HLA-I LOH positive status; or treating the subject with an IO therapy if the subject is determined to have an HLA-I LOH non-positive status.

Embodiments of the present disclosure comprise methods for selecting a treatment for a subject having a cancer, the method comprising: receiving, at one or more processors, a plurality of sequence reads corresponding to a plurality of genomic segments in a sample from a subject, wherein the plurality of sequence reads are aligned with a reference sequence; determining: whether a number of sequence reads in the plurality of sequence reads exceeds a predetermined sequence read threshold; whether the plurality of genomic segments are germline heterozygous at one or more HLA-I gene loci in the sample; and whether a tumor content of the sample is above a predetermined tumor content threshold; when the plurality of genomic segments are germline heterozygous, the number of sequence reads in the plurality of sequence reads exceeds the predetermined sequence read threshold, and the tumor content is above the predetermined tumor content threshold: determining a gene minor allele frequency (MAF) of the sample and a segment MAF of the sample; and determining a loss of heterozygosity (LOH) status based on the gene MAF of the sample, the segment MAF of the sample, and the tumor content of the sample, wherein an HLA-I LOH positive status is determined if the tumor content is above the predetermined tumor content threshold and the gene MAF is below a first gene threshold and the segment MAF is below a first segment threshold; and identifying the subject for treatment with a non-IO therapy if the subject is determined to have the HLA-I LOH positive status.

Embodiments of the present disclosure comprise methods for identifying a subject having a cancer for treatment with an immune oncology (IO) therapy, the method comprising: receiving, at one or more processors, a plurality of sequence reads corresponding to a plurality of genomic segments in a sample from a subject, wherein the plurality of sequence reads are aligned with a reference sequence; determining: whether a number of sequence reads in the plurality of sequence reads exceeds a predetermined sequence read threshold; whether the plurality of genomic segments are germline heterozygous at one or more HLA-I gene loci in the sample; and whether a tumor content of the sample is above a predetermined tumor content threshold; when the plurality of genomic segments are germline heterozygous, the number of sequence reads in the plurality of sequence reads exceeds the predetermined sequence read threshold, and the tumor content is above the predetermined tumor content threshold: determining a gene minor allele frequency (MAF) of the sample and a segment MAF of the sample; and determining a loss of heterozygosity (LOH) status based on the gene MAF of the sample, the segment MAF of the sample, and the tumor content of the sample, wherein an HLA-I LOH positive status is determined if the tumor content is above the predetermined tumor content threshold and the gene MAF is below a first gene threshold and the segment MAF is below a first segment threshold; and identifying the subject for treatment with a non-IO therapy if the subject is determined to have the HLA-I LOH positive status.

Embodiments of the present disclosure comprise methods of identifying one or more treatment options for a subject having a cancer, the method comprising: receiving, at one or more processors, a plurality of sequence reads corresponding to a plurality of genomic segments in a sample from a subject, wherein the plurality of sequence reads are aligned with a reference sequence; determining: whether a number of sequence reads in the plurality of sequence reads exceeds a predetermined sequence read threshold; whether the plurality of genomic segments are germline heterozygous at one or more HLA-I gene loci in the sample; and whether a tumor content of the sample is above a predetermined tumor content threshold; when the plurality of genomic segments are germline heterozygous, the number of sequence reads in the plurality of sequence reads exceeds the predetermined sequence read threshold, and the tumor content is above the predetermined tumor content threshold: determining a gene minor allele frequency (MAF) of the sample and a segment MAF of the sample; and determining a loss of heterozygosity (LOH) status based on the gene MAF of the sample, the segment MAF of the sample, and the tumor content of the sample, wherein an HLA-I LOH positive status is determined if the tumor content is above the predetermined tumor content threshold and the gene MAF is below a first gene threshold and the segment MAF is below a first segment threshold; generating a report comprising one or more treatment options identified for the subject based at least in part on the HLA-I LOH status determined for the sample, wherein: the subject is identified as one who may benefit from treatment with a non-IO therapy if the subject is determined to have the HLA-I LOH positive status.

Embodiments of the present disclosure comprise methods of stratifying a subject having cancer for treatment with an immuno-oncology (IO) therapy, the method comprising receiving, at one or more processors, a plurality of sequence reads corresponding to a plurality of genomic segments in a sample from a subject, wherein the plurality of sequence reads are aligned with a reference sequence; determining: whether a number of sequence reads in the plurality of sequence reads exceeds a predetermined sequence read threshold; whether the plurality of genomic segments are germline heterozygous at one or more HLA-I gene loci in the sample; and whether a tumor content of the sample is above a predetermined tumor content threshold; when the plurality of genomic segments are germline heterozygous, the number of sequence reads in the plurality of sequence reads exceeds the predetermined sequence read threshold, and the tumor content is above the predetermined tumor content threshold: determining a gene minor allele frequency (MAF) of the sample and a segment MAF of the sample; and determining a loss of heterozygosity (LOH) status based on the gene MAF of the sample, the segment MAF of the sample, and the tumor content of the sample, wherein an HLA-I LOH positive status is determined if the tumor content is above the predetermined tumor content threshold and the gene MAF is below a first gene threshold and the segment MAF is below a first segment threshold; and treating the subject with a non-IO therapy if the subject is determined to have the HLA-I LOH positive status.

Embodiments of the present disclosure comprise methods of predicting survival of a subject having cancer, the method comprising: receiving, at one or more processors, a plurality of sequence reads corresponding to a plurality of genomic segments in a sample from a subject, wherein the plurality of sequence reads are aligned with a reference sequence; determining: whether a number of sequence reads in the plurality of sequence reads exceeds a predetermined sequence read threshold; whether the plurality of genomic segments are germline heterozygous at one or more HLA-I gene loci in the sample; and whether a tumor content of the sample is above a predetermined tumor content threshold; when the plurality of genomic segments are germline heterozygous, the number of sequence reads in the plurality of sequence reads exceeds the predetermined sequence read threshold, and the tumor content is above the predetermined tumor content threshold: determining a gene minor allele frequency (MAF) of the sample and a segment MAF of the sample; and determining a loss of heterozygosity (LOH) status based on the gene MAF of the sample, the segment MAF of the sample, and the tumor content of the sample, wherein an HLA-I LOH positive status is determined if the tumor content is above the predetermined tumor content threshold and the gene MAF is below a first gene threshold and the segment MAF is below a first segment threshold; wherein: if the subject is determined to have the HLA-I LOH positive status, the subject is predicted to have shorter survival when treated with an IO therapy, as compared to a subject that was determined to have an HLA-I LOH non-positive status.

Embodiments of the present disclosure comprise methods of predicting survival of a subject having cancer, the method comprising: receiving, at one or more processors, a plurality of sequence reads corresponding to a plurality of genomic segments in a sample from a subject, wherein the plurality of sequence reads are aligned with a reference sequence; determining: whether a number of sequence reads in the plurality of sequence reads exceeds a predetermined sequence read threshold; whether the plurality of genomic segments are germline heterozygous at one or more HLA-I gene loci in the sample; and whether a tumor content of the sample is above a predetermined tumor content threshold; when the plurality of genomic segments are germline heterozygous, the number of sequence reads in the plurality of sequence reads exceeds the predetermined sequence read threshold, and the tumor content is above the predetermined tumor content threshold: determining a gene minor allele frequency (MAF) of the sample and a segment MAF of the sample; and determining a loss of heterozygosity (LOH) status based on the gene MAF of the sample, the segment MAF of the sample, and the tumor content of the sample, wherein an HLA-I LOH positive status is determined if the tumor content is above the predetermined tumor content threshold and the gene MAF is below a first gene threshold and the segment MAF is below a first segment threshold; wherein: if the subject is determined to have the HLA-I LOH positive status, the subject is predicted to have longer survival when treated with a non-immuno-oncology (IO) therapy, as compared to a subject that was treated with an IO therapy.

In one or more embodiments, the methods described above further comprise determining a tumor mutational burden (TMB) in the sample from the subject, wherein predicted survival is further based on the TMB. In one or more embodiments of the methods described above, the subject is determined to have a TMB of at least about 4 to 100 mutations/Mb, about 4 to 30 mutations/Mb, 8 to 100 mutations/Mb, 8 to 30 mutations/Mb, 10 to 20 mutations/Mb, less than 4 mutations/Mb, or less than 8 mutations/Mb. In one or more embodiments of the methods described above, the TMB is at least about 5 mutations/Mb, is at least about 10 mutations/Mb, at least about 12 mutations/Mb, at least about 16 mutations/Mb, at least about 20 mutations/Mb, or at least about 30 mutations/Mb. In one or more embodiments of the methods described above, the TMB is determined based on between about 100 kb to about 10 Mb. In one or more embodiments of the methods described above, the TMB is determined based on between about 0.8 Mb to about 1.1 Mb.

In one or more embodiments of the methods described above, the IO therapy comprises a single IO agent or multiple IO agents. In one or more embodiments of the methods described above, the IO therapy comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a cellular therapy, a treatment for cancer being tested in a clinical trial, an immunotherapy, or any combination thereof.

In one or more embodiments of the methods described above, the IO therapy comprises an immune checkpoint inhibitor. In one or more embodiments, the immune checkpoint inhibitor is a PD-1 inhibitor. In one or more embodiments, the immune checkpoint inhibitor comprises one or more of nivolumab, pembrolizumab, cemiplimab, or dostarlimab. In one or more embodiments, the immune checkpoint inhibitor is a PD-L1-inhibitor. In one or more embodiments, the immune checkpoint inhibitor comprises one or more of atezolizumab, avelumab, or durvalumab. In one or more embodiments, the immune checkpoint inhibitor is a CTLA-4 inhibitor. In one or more embodiments, the CTLA-4 inhibitor comprises ipilimumab. In one or more embodiments, the nucleic acid comprises a double-stranded RNA (dsRNA), a small interfering RNA (siRNA), or a small hairpin RNA (shRNA). In one or more embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In one or more embodiments, the methods described above further comprise treating the subject determined to have an HLA-I LOH non-positive status with the IO therapy. In one or more embodiments of the methods described above, the non-IO therapy comprises one or more of a small molecule inhibitor, a chemotherapeutic agent, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

In one or more embodiments, the chemotherapeutic agent comprises one or more of an alkylating agent, an alkyl sulfonates aziridine, an ethylenimine, a methylamelamine, an acetogenin, a camptothecin, a bryostatin, a callystatin, CC-1065, a cryptophycin, aa dolastatin, a duocarmycin, a eleutherobin, a pancratistatin, a sarcodictyin, a spongistatin, a nitrogen mustard, a nitrosureas, an antibiotic, a dynemicin, a bisphosphonate, an esperamicina a neocarzinostatin chromophore or a related chromoprotein enediyne antiobiotic chromophore, an anti-metabolite, a folic acid analogue, a purine analog, a pyrimidine analog, an androgens, an anti-adrenal, a folic acid replenisher, aldophosphamide glycoside, aminolevulinic acid, eniluracil, amsacrine, bestrabucil, bisantrene, edatraxate, defofamine, demecolcine, diaziquone, elformithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, a PSK polysaccharide complex, razoxane, rhizoxin, sizofiran, spirogermanium, tenuazonic acid, triaziquone, 2,2′,2″-trichlorotriethylamine, a trichothecene, urethan, vindesine, dacarbazine, mannomustine, mitobronitol, mitolactol, pipobroman, gacytosine, arabinoside (“Ara-C”), cyclophosphamide, a taxoid, 6-thioguanine, mercaptopurine, a platinum coordination complex, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluorometlhylomithine (DMFO), a retinoid, capecitabine, carboplatin, procarbazine, plicomycin, gemcitabine, navelbine, farnesyl-protein transferase inhibitors, transplatinum, or any combination thereof.

In one or more embodiments, the methods described above further comprise treating the subject determined to have an HLA-I LOH positive status with the non-IO therapy. In one or more embodiments, the methods described above further comprise treating the subject with an additional anti-cancer therapy. In one or more embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

In one or more embodiments of the methods described above, the survival is a progression-free survival, an overall survival, a disease-free survival (DFS), an objective response rate (ORR), a time to tumor progression (TTP), a time to treatment failure (TTF), a durable complete response (DCR), or a time to next treatment (TTNT).

In one or more embodiments of the methods described above, the sample comprises a tissue biopsy sample or a liquid biopsy sample. In such embodiments, the sample is a tissue biopsy and comprises a tumor biopsy, tumor specimen, or circulating tumor cells. In one or more embodiments, the sample is a liquid biopsy sample and comprises blood, serum, plasma, cerebrospinal fluid, sputum, stool, urine, or saliva.

In one or more embodiments of the methods described above, the sample comprises cells and/or nucleic acids from the cancer. In such embodiments, the sample comprises mRNA, DNA, circulating tumor DNA (ctDNA), cell-free DNA, cell-free RNA from the cancer, or any combination thereof. In one or more embodiments, the sample is a liquid biopsy sample and comprises circulating tumor cells (CTCs). In one or more embodiments, the sample is a liquid biopsy sample and comprises cell-free DNA (cfDNA), circulating tumor DNA (ctDNA), or any combination thereof.

In one or more embodiments of the methods described above, the LOH status of the gene is determined based on sequencing read data derived from sequencing the sample from the subject. In such embodiments, the sequencing comprises use of a massively parallel sequencing (MPS) technique, whole genome sequencing (WGS), whole exome sequencing, targeted sequencing, direct sequencing, next-generation sequencing (NGS), or a Sanger sequencing technique. In one or more embodiments, the sequencing comprises: providing a plurality of nucleic acid molecules obtained from the sample, wherein the plurality of nucleic acid molecules comprises a mixture of tumor nucleic acid molecules and non-tumor nucleic acid molecules; optionally, ligating one or more adapters onto one or more nucleic acid molecules from the plurality of nucleic acid molecules; amplifying nucleic acid molecules from the plurality of nucleic acid molecules; optionally, capturing nucleic acid molecules from the amplified nucleic acid molecules, wherein the captured nucleic acid molecules are captured from the amplified nucleic acid molecules by hybridization to one or more bait molecules; and sequencing, by a sequencer, the captured nucleic acid molecules to obtain a plurality of sequence reads corresponding to one or more genomic loci within a subgenomic interval in the sample.

In one or more embodiments, the adapters comprise one or more of amplification primer sequences, flow cell adapter hybridization sequences, unique molecular identifier sequences, substrate adapter sequences, or sample index sequences. In one or more embodiments, amplifying nucleic acid molecules comprises performing a polymerase chain reaction (PCR) technique, a non-PCR amplification technique, or an isothermal amplification technique. In one or more embodiments, the one or more bait molecules comprise one or more nucleic acid molecules, each comprising a region that is complementary to a region of a captured nucleic acid molecule. In such embodiments, the one or more bait molecules each comprise a capture moiety. In such embodiments, the capture moiety is biotin.

In one or more embodiments of the methods described above, the cancer is a B cell cancer, a melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In one or more embodiments of the methods described above, the subject is a human. In one or more embodiments of the methods described above, the subject has previously been treated with an anti-cancer therapy. In such embodiments, the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1A provides a non-limiting example of a process for determining an HLA-I LOH status according to embodiments of the present disclosure.

FIG. 1B provides a non-limiting example of a process for determining an HLA-I LOH status according to embodiments of the present disclosure.

FIG. 2 provides a non-limiting example of a process for determining a copy number value according to embodiments of the present disclosure.

FIG. 3 provides a non-limiting example of a process for determining a copy number value according to embodiments of the present disclosure.

FIG. 4 provides a non-limiting example of a process for determining a copy number value according to embodiments of the present disclosure.

FIG. 5 provides a non-limiting example of a process for determining a copy number value according to embodiments of the present disclosure.

FIG. 6 provides a non-limiting example of a process for determining an HLA-I LOH status according to embodiments of the present disclosure.

FIGS. 7A and 7B provide a non-limiting example of a process for determining a tumor content of the sample according to embodiments of the present disclosure.

FIG. 8 provides a non-limiting example of a process for determining a tumor content of the sample according to embodiments of the present disclosure.

FIG. 9 provides a non-limiting example of a process for determining a tumor content of the sample according to embodiments of the present disclosure.

FIG. 10 provides a non-limiting example of a process for determining a tumor content of the sample according to embodiments of the present disclosure.

FIG. 11 provides a non-limiting example of a process for determining a tumor content of the sample according to embodiments of the present disclosure.

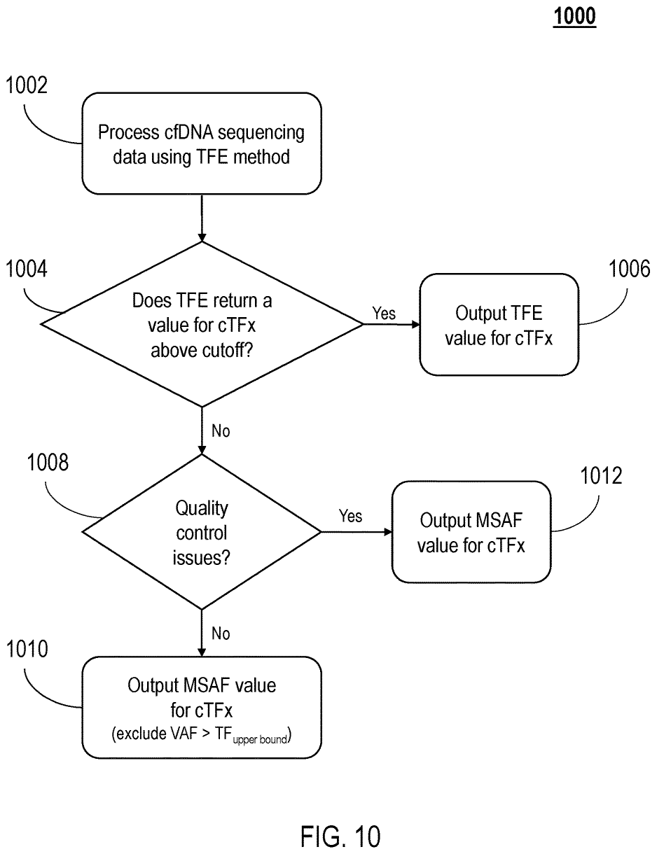

FIG. 12 depicts an exemplary computing device or system in accordance with one embodiment of the present disclosure.

FIG. 13 depicts an exemplary computer system or computer network, in accordance with some instances of the systems described herein.

FIG. 14 depicts an exemplary plot illustrating the overall survival of individuals grouped by a predicted HLA-I LOH status, according to embodiments of the present disclosure.

FIG. 15 depicts an exemplary plot illustrating the overall survival of individuals grouped by a predicted HLA-I LOH status, according to embodiments of the present disclosure.

FIG. 16 depicts an exemplary plot illustrating the progression-free survival of individuals grouped by a predicted HLA-I LOH status, according to embodiments of the present disclosure.

### DETAILED DESCRIPTION

Methods and systems for determining a loss of heterozygosity (LOH) status of one or more HLA-I genes are described. Loss of heterozygosity (LOH) of the HLA class I (HLA-I) genes, e.g., HLA-A, HLA-B, or HLA-C, is understood as a mechanism of immune evasion and has found to be correlated with worse outcomes in patients treated with immune checkpoint inhibitors. In one study, it was determined that HLA-I LOH occurred in 17% of patients and was a significant negative predictor of overall survival in non-squamous non-small cell lung cancer (NSCLC) patients treated with second line immune checkpoint inhibitor monotherapy. See Montesion et al. (2020) “Somatic HLA Class I Loss Is a Widespread Mechanism of Immune Evasion Which Refines the Use of Tumor Mutational Burden as a Biomarker of Checkpoint Inhibitor Response”, https://pubmed.ncbi.nlm.nih.gov/33127846/. This study used a combination of allelic imbalance and copy number modeling to determine whether a tumor sample has a positive HLA-I LOH status (e.g., indicative of HLA-I LOH) or a non-positive HLA-I LOH status (e.g., suggestive of HLA-I heterozygosity).

While using both allelic imbalance and copy number modeling as input may provide confidence in the HLA-I LOH status call assigned to a sample, in some instances, information regarding both allelic imbalance and copy number modeling may not be available. For example, certain diagnostic tests may not provide information regarding both allelic imbalance and copy number modeling. For example, allelic imbalance information may not be available in tests that do not bait for the HLA-I genes. As another example, copy number modeling data may not be available for liquid biopsy tests. In such instances, the ability to determine an HLA-I LOH status may still be valuable. Accordingly, there is a need for methods to determine HLA-I LOH status of a sample in instances where information regarding both allelic imbalance and copy number modeling may not be available.

Disclosed herein are methods and systems for identifying HLA-I loss of heterozygosity (LOH) of a sample. One or more embodiments of this disclosure provide methods for identifying HLA-I LOH when allelic imbalance information is available, but copy number variation information is unavailable. One or more embodiments of this disclosure provide methods for identifying HLA-I LOH when copy number variation information is available, but allelic imbalance information is unavailable. Accordingly, embodiments of this disclosure provide systems and methods for determining an HLA-I LOH status of a sample when both allelic imbalance information and copy number modeling information are not readily available.

In some instances, for example, methods are described that comprise identifying a plurality of genomic segments in a sample; selecting from the plurality of genomic segments, one or more genomic segments that overlap with one or more HLA-I genes in the sample; determining whether a tumor purity of the sample is in a predetermined range; when the tumor purity of the sample is in the predetermined range, obtaining a copy number value of the one or more selected genomic segments; and determining a loss of heterozygosity (LOH) status of the one or more HLA-I genes based on the copy number value.

In one or more embodiments, the method for determining a LOH status of the one or more HLA-I genes further comprises performing a quality control procedure prior to obtaining the copy number value; and foregoing determining the LOH status of the one or more HLA-I genes in accordance with a determination that the sample is associated with a quality control issue. In such embodiments, wherein the quality control issue includes a sample contamination, a confirmed transplant, a noisy copy number, or a combination thereof.

In one or more embodiments, the sample may be at least one of a tissue biopsy sample or a liquid biopsy sample. In such embodiments, the biopsy sample may comprise a normal tissue and a tumor tissue.

In one or more embodiments, the predetermined range comprises a tumor purity between 20-95%. In one or more embodiments, the LOH status may be determined without baiting the one or more HLA-I genes. In one or more embodiments, the one or more HLA-I genes comprise an HLA-A gene, an HLA-B gene, an HLA-C gene, or a combination thereof.

In one or more embodiments, determining the loss of heterozygosity (LOH) status can comprise: determining the LOH status to be indeterminate if the copy number value is at about a first threshold; determining the LOH status to be positive if the copy number value is at about a second threshold; and determining the LOH status to be negative if the copy number value is above a third threshold. In such embodiments, the first threshold can be about 0, the second threshold can be about 1, and the third threshold can be about 2. In one or more embodiments, when the copy number value is at the third threshold, the system can determine whether the LOH status is a neutral LOH; and the system can determine the LOH status to be: (1) non-positive (e.g., negative) if the LOH status does not correspond to the neutral LOH, and (2) positive if the LOH status corresponds to the neutral LOH.

The disclosed methods and systems can determine an HLA-I LOH status of a sample when either the allelic imbalance information or copy number modeling information is not readily available.

Embodiments of the present disclosure further comprise a system comprising: one or more processors; and a memory communicatively coupled to the one or more processors and configured to store instructions that, when executed by the one or more processors, cause the system to: identify a plurality of genomic segments in a sample; select from the plurality of genomic segments, one or more genomic segments that overlap with one or more HLA-I genes in the sample; determine whether a tumor purity of the sample is in a predetermined range; when the tumor purity of the sample is in the predetermined range, obtain a copy number value of the one or more selected genomic segments; determine a loss of heterozygosity (LOH) status of the one or more HLA-I genes based on the copy number value.

Embodiments of the present disclosure further comprise a non-transitory computer-readable storage medium storing one or more programs, the one or more programs comprising instructions, which when executed by one or more processors of a system, cause the system to: identify a plurality of genomic segments in a sample; select from the plurality of genomic segments, one or more genomic segments that overlap with one or more HLA-I genes in the sample; determine whether a tumor purity of the sample is in a predetermined range; when the tumor purity of the sample is in the predetermined range, obtain a copy number value of the one or more selected genomic segments; and determine a loss of heterozygosity (LOH) status of the one or more HLA-I genes based on the copy number value.

Embodiments of the present disclosure can further comprise methods comprising: identifying, using one or more processors, a plurality of genomic segments in a sample; selecting from the plurality of genomic segments, one or more genomic segments that overlap with one or more HLA-I genes in the sample without baiting the one or more HLA-I genes; determining whether a tumor purity of the sample is in a predetermined range; when the tumor purity of the sample is in the predetermined range, obtaining a copy number value of the one or more selected genomic segments; determining a loss of heterozygosity (LOH) status of the one or more HLA-I genes.

Embodiments of the present disclosure can further comprise methods comprising: receiving, at the one or more processors, one or more reads corresponding to a plurality of genomic segments in a sample, wherein the one or more reads are aligned with a reference sequence; determining, using the one or more processors: whether a number of one or more reads exceeds a predetermined read threshold; whether the plurality of genomic segments are germline heterozygous at one or more HLA-I genes in the sample; and whether a tumor content of the sample is above a predetermined tumor content threshold; when the plurality of genomic segments are germline heterozygous, the number of reads in the one or more reads exceeds the predetermined read threshold, and the tumor content is above the predetermined tumor content threshold: determining a gene minor allele frequency (MAF) of the sample and a segment MAF of the sample; and determining a loss of heterozygosity (LOH) status based on the gene MAF of the sample, the segment MAF of the sample, and the tumor content of the sample.

In one or more embodiments of the present disclosure, the sample comprises at least one of a tissue biopsy sample, a liquid biopsy sample, or a normal control. In one or more embodiments of the present disclosure, the tumor content corresponds to a maximum somatic allele frequency (MSAF). In one or more embodiments of the present disclosure, the tumor content corresponds to a tumor purity. In one or more embodiments of the present disclosure, the one or more HLA-I genes comprise an HLA-A gene, an HLA-B gene, an HLA-C gene, or a combination thereof.

In one or more embodiments of the present disclosure the methods can further comprise sequencing the one or more reads corresponding to the plurality of genomic segments in the sample that have been baited for the one or more HLA-I genes.

In one or more embodiments of the present disclosure the methods can further comprise: aligning the one or more reads corresponding to a plurality of genomic segments in the sample to the reference sequence.

In one or more embodiments of the present disclosure determining whether the plurality of genomic segments in the sample are germline heterozygous at the one or more HLA-I genes in the sample comprises determining an allele fraction of the sample at the one or more HLA-I genes is about 0.5.

In one or more embodiments of the present disclosure, determining whether the number of the one or more reads in the sample exceeds the predetermined read threshold comprises: aligning the one or more reads corresponding to the plurality of genomic segments in the sample to the reference sequence, the reference sequence comprising one or more reads of an HLA allele of the one or more HLA-I genes; and determining the number of the one or more reads corresponding to the plurality of genomic segments aligned with the references sequence; and comparing the number to the predetermined read threshold. In one or more embodiments of the present disclosure, the read threshold is one of 1000 reads, 1100 reads, 1200 reads, 1300 reads, 1400 reads, 1500 reads, 1600 reads, 1700 reads, 1800 reads, 1900 reads, 2000 reads, 2100 reads, 2200 reads, 2300 reads, 2400 reads, 2500 reads, 2600 reads, 2700 reads, 2800 reads, 2900 reads, or 3000 reads.

In one or more embodiments of the present disclosure, the tumor content threshold corresponds to a tumor content of 1%, 5%, or 10%.

Embodiments of the present disclosure further comprise systems for determining the HLA-I LOH status of a sample, the system comprising: one or more processors; and a memory communicatively coupled to the one or more processors and configured to store instructions that, when executed by the one or more processors, cause the system to: receive, at the one or more processors, one or more reads corresponding to a plurality of genomic segments in a sample, wherein the one or more reads are aligned with a reference sequence; determine, using the one or more processors: whether a number of one or more reads exceeds a predetermined read threshold; whether the plurality of genomic segments are germline heterozygous at one or more HLA-I genes in the sample; and whether a tumor content of the sample is above a predetermined tumor content threshold; when the plurality of genomic segments are germline heterozygous, the number of reads in the one or more reads exceeds the predetermined read threshold, and the tumor content is above the predetermined tumor content threshold: determine a gene minor allele frequency (MAF) of the sample and a segment MAF of the sample; and determine a loss of heterozygosity (LOH) status of the sample based on the gene MAF of the sample, the segment MAF of the sample, and the tumor content of the sample.

Embodiments of the present disclosure further comprise non-transitory computer-readable storage mediums storing one or more programs, the one or more programs comprising instructions, which when executed by one or more processors of a system, cause the system to: receive, at the one or more processors, one or more reads corresponding to a plurality of genomic segments in a sample, wherein the one or more reads are aligned with a reference sequence; determine, using the one or more processors: whether a number of one or more reads exceeds a predetermined read threshold; whether the plurality of genomic segments are germline heterozygous at one or more HLA-I genes in the sample; and whether a tumor content of the sample is above a predetermined tumor content threshold; when the plurality of genomic segments are germline heterozygous, the number of reads in the one or more reads exceeds the predetermined read threshold, and the tumor content is above the predetermined tumor content threshold: determine a gene minor allele frequency (MAF) of the sample and a segment MAF of the sample; and determine a loss of heterozygosity (LOH) status of the sample based on the gene MAF of the sample, the segment MAF of the sample, and the tumor content of the sample.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The section headings used herein are for organizational purposes only and are not to be construed as limiting the subject matter described.

### Methods for Determining a Loss of Heterozygosity of HLA-I Genes Using Copy-Number Data

One or more embodiments of the present disclosure provide methods for determining HLA-I loss of heterozygosity (LOH) of a sample. One or more embodiments of this disclosure provide methods for identifying HLA-I LOH when copy number variation information is available. One or more embodiments of this disclosure provide methods for identifying HLA-I LOH when copy number variation information is available, but allelic imbalance information is unavailable.

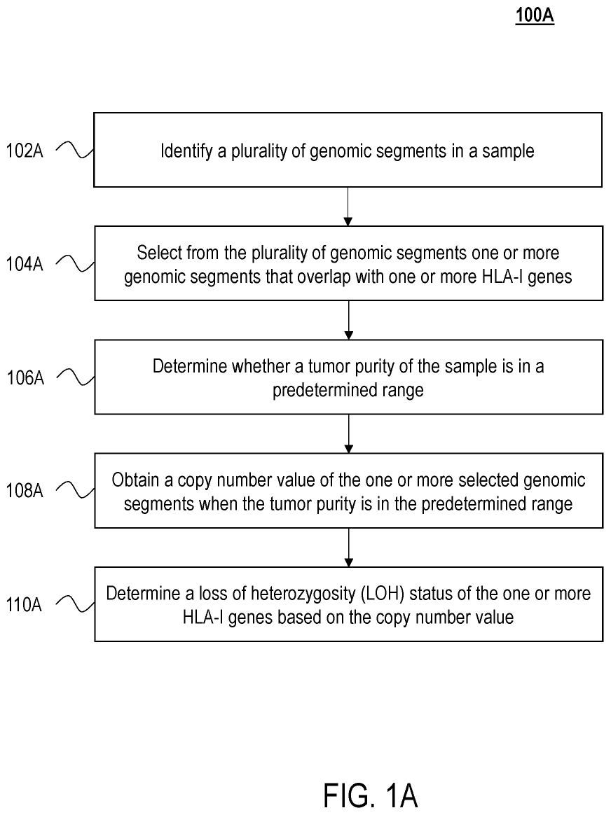

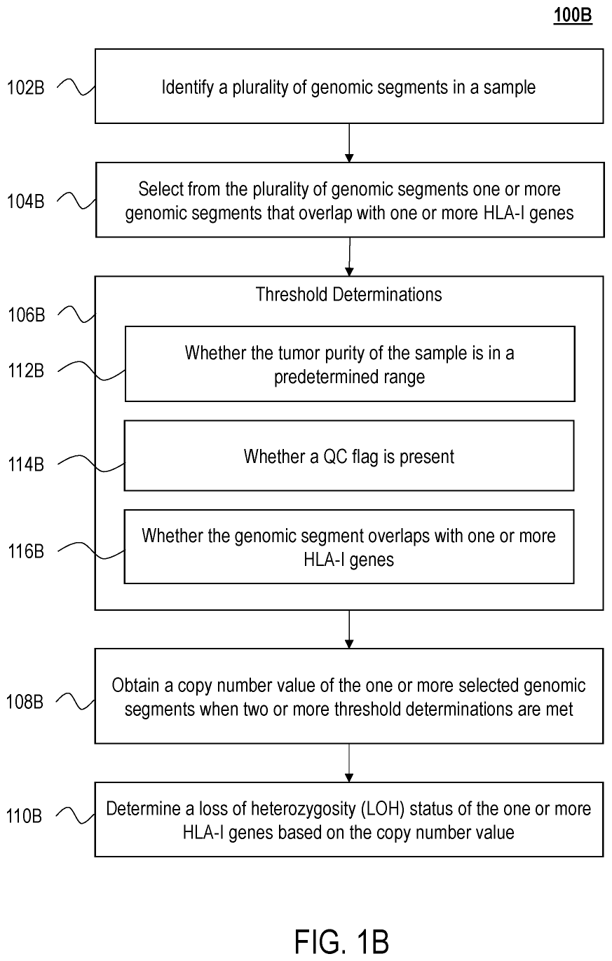

FIG. 1A and FIG. 1B provide non-limiting examples of processes 100A and 100B for determining an HLA-I LOH status of a sample. In one or more examples, processes 100A and 100B can determine the HLA-I LOH status of the sample based on copy number information without relying on allelic imbalance information of the sample. In one or more examples, processes 100A and 100B can be applied to at least one of a tissue biopsy sample, a liquid biopsy sample, or a normal control. In one or more examples, processes 100A and 100B can be applied to samples, where the HLA-I genes are not baited.

Processes 100A and 100B can be performed, for example, using one or more electronic devices implementing a software platform. In some examples, processes 100A and 100B are performed using a client-server system, and the blocks of processes 100A and 100B are divided up in any manner between the server and a client device. In other examples, the blocks of processes 100A and 100B are divided up between the server and multiple client devices. Thus, while portions of processes 100A and 100B are described herein as being performed by particular devices of a client-server system, it will be appreciated that processes 100A and 100B are not so limited. In other examples, processes 100A and 100B is performed using only a client device or only multiple client devices. In processes 100A and 100B, some blocks are, optionally, combined, the order of some blocks is, optionally, changed, and some blocks are, optionally, omitted. In some examples, additional steps may be performed in combination with processes 100A and 100B. Accordingly, the operations as illustrated (and described in greater detail below) are exemplary by nature and, as such, should not be viewed as limiting.

At step 102A in FIG. 1A, the system can identify a plurality of genomic segments in a sample. In one or more examples, the system can receive sequence data associated with a sample from the patient and the plurality of genomic segments may be identified based on the sequence read data. In one or more examples, the sequence read data can be derived from, e.g., targeted exome sequencing or whole exome sequencing.

At step 104A in FIG. 1A, the system can select from the plurality of genomic segments one or more genomic segments that overlap with one or more HLA-I genes. In one or more examples, the HLA-I genes of interest may include HLA-A, HLA-B, and/or HLA-C. In one or more examples, a genomic segment may be selected if the genomic segment overlaps with two or more HLA-I genes (e.g., two or more of HLA-A, HLA-B, and/or HLA-C). In one or more examples, a genomic segment may be selected if the genomic segment overlaps with three or more HLA-I genes (e.g., HLA-A, HLA-B, and HLA-C). In one or more examples, the system may not bait for the HLA-I genes.

At step 106A in FIG. 1A, the system can determine whether a tumor purity of the sample is in a predetermined range. For example, the system may obtain a tumor purity via a computational pipeline for processing nucleic acid sequencing data. In one or more examples, the desirable range may comprise a tumor purity of 20% to 95%. In one or more examples, the system may not be able to assess whether somatic LOH occurs for a sample with a tumor purity below 20% due to the large presence of non-tumor tissue.

In one or more examples, the system can determine the tumor purity based on sequence read data of the sample. For example, the system may obtain a tumor purity via a computational pipeline. In one or more examples the tumor purity may be based on a genomic copy number profile of the sample. The genomic copy number profile may be based on the sequence read data corresponding to genomic coverage and allele frequencies at a plurality of single nucleotide polymorphisms (SNPs). The pipeline may segment and model the SNPs to determine the tumor purity of the sample. In one or more examples, the system can receive the tumor purity based on one or more diagnostic test results.

If the tumor purity of the sample is determined to be outside the predetermined range, then the sample may be discarded such that the system does not determine an HLA-I LOH status of the sample. In one or more examples, if the tumor purity is less than 20% or greater than 95%, the sample may be discarded by the system such that the system does not determine an HLA-I LOH status of the sample. By ensuring that the tumor purity is within the predefined range, the system ensures that there is a sufficient amount of tumor and non-tumor cells within the sample to provide a reliable HLA-I LOH determination. If there is a lack of tumor cells in the sample, the system may not be able to identify whether somatic HLA-I LOH has occurred. If there is a lack of non-tumor cells, the system may not be able to identify whether the homozygosity observed at the HLA-I locus is germline or somatic. If the homozygosity at the HLA-I locus is germline, then the sample would not be considered HLA-I LOH.

At step 108A in FIG. 1A, when the tumor purity is determined to be in the predetermined range, the system can obtain a copy number value of the one or more selected genomic segments. In one or more examples, the copy number value can correspond, but not be limited, to a copy number alteration (CNA) score or a zygosity score. While methods for determining the CNA score and the zygosity score are described below, a skilled artisan will understand that other methods for determining the copy number value may be used without departing from the scope of this disclosure.

In one or more examples, the CNA score can be indicative of a number of copy number variations of the segment. In one or more examples, the CNA score may be an integer. In one or more examples, the zygosity score can be indicative of a zygosity of the sample. In one or more examples, the zygosity score may be a number between zero and one. In one or more examples, the zygosity may be based on the CNA score.

At step 110A in FIG. 1A, the system can determine a LOH status of the one or more HLA-I genes based on the copy number value. For example, the HLA-I LOH status may be based on a CNA score. In one or more examples, the HLA-I LOH status may be based on a zygosity score.

In one or more examples, if the copy number value corresponds to a CNA score, the one or more genomic segments may be determined to have a positive LOH status if the CNA score is about one. In such examples, the CNA score of about one indicates that a single copy of the gene is present, which suggests a loss of heterozygosity. In one or more examples, the one or more selected genomic segments may be determined to have a non-positive (e.g., negative) LOH status if the CNA score is about zero or greater than two. In such examples, the CNA score of about zero indicates that there are no copies of the gene present, which is suggestive of a deep deletion. In such examples, a CNA score of greater two indicates that there are multiple copies of the gene in the segment, which is not suggestive of a positive LOH status.

In one or more examples, the one or more selected genomic segments may be determined to have an indeterminate LOH status if the copy number value is about two. In such examples, the CNA score of about two may indicate that the sample is heterozygous, at least because there are two copies of the gene present. In some examples, a CNA score of about two may be associated with a neutral LOH, wherein one chromosome of the segment has lost a copy of the gene, but the other chromosome of the segment has gained two copies of the gene. In one or more examples, if the HLA-I LOH status is determined to be indeterminate, the system may further determine whether the copy number value corresponds to a neutral LOH. If the system determines that the segment is associated with a neutral LOH, then the system may determine a positive HLA-I LOH status. If the system determines that the segment is not associated with a neutral LOH, then the system may determine a non-positive (e.g., negative) HLA-I LOH status.

In one or more examples, the HLA-I LOH status may be based on a zygosity score. In such examples, the one or more selected genomic segments may be determined to have a positive LOH status if the zygosity score is about one. In one or more examples, the one or more selected genomic segments may be determined to have a non-positive (e.g., negative) LOH status if the zygosity score is about 0.

FIG. 1B provides a non-limiting example of a process 100B for determining an HLA-I LOH status of a sample. At step 102B in FIG. 1B, the system can identify a plurality of genomic segments in a sample. In one or more examples, step 102B can correspond to step 102A in FIG. 1A.

At step 104B in FIG. 1B, the system can select from the plurality of genomic segments one or more genomic segments that overlap with one or more HLA-I genes. In one or more examples, the HLA-I genes of interest may include HLA-A, HLA-B, and HLA-C. In one or more examples, the system may not bait for the HLA-I genes. In one or more examples, step 104B can correspond to step 104A in FIG. 1A.

At step 106B in FIG. 1B, the system can perform one or more threshold determinations. For example, the system can determine: whether the tumor purity of the sample is in a predetermined range at step 112B, whether a quality control flag is present at step 114B, and whether the genomic segment overlaps with one or more HLA-I genes at step 116B.

At step 112B in FIG. 1B, the system can determine whether the tumor purity of the sample is in a predetermined range. In one or more examples, step 112B can correspond to step 1016A in FIG. 1A.

At step 114B in FIG. 1B, the system can determine whether a quality control flag is present. In one or more examples, the quality control flags can include, but not be limited to but are not limited to, an average sequence coverage for the sample, a minimum average sequence coverage for the sample, an allele coverage at each of the corresponding loci in the plurality of loci, a minimum allele coverage at each of the corresponding loci in the plurality of loci, a degree of nucleic acid contamination in the sample (determined, e.g., by quantifying aberrations in SNP allele frequencies), a maximum degree of nucleic acid contamination in the sample, a number of single nucleotide polymorphism (SNP) loci within the plurality of loci examined a minimum number of single nucleotide polymorphism (SNP) loci within the plurality of loci examined, or any combination thereof.

At step 116B in FIG. 1B, the system can determine whether the genomic segment overlaps with one or more HLA-I genes. In one or more examples, the threshold determination that the genomic segment overlaps with one or more HLA-I genes may be met if the segment overlaps with one or more of the HLA-A, HLA-B, or HLA-C. In one or more examples, this threshold determination may be met if the segment overlaps with two or more of the HLA-A, HLA-B, and/or HLA-C. In one or more examples, this threshold determination may be met if the segment overlaps with each of HLA-A, HLA-B, and HLA-C.

At step 108B in FIG. 1B, if one or more of the threshold determinations are met, the system can obtain a copy number value of the one or more selected genomic segments. In one or more examples, the copy number value can correspond, but not be limited, to a copy number alteration (CNA) score or a zygosity score. While methods for determining the CNA score and the zygosity score are described below, a skilled artisan will understand that other methods for determining the copy number value may be used without departing from the scope of this disclosure. In one or more examples, the process 100B may not proceed unless at least two of the threshold determinations performed at steps 112B, 114B, and 116B are met. In one or more examples, the process 100B may not proceed unless each of the threshold determinations performed at steps 112B, 114B, and 116B are met.

At step 110B in FIG. 1B, the system can determine a LOH status of the one or more HLA-I genes based on the copy number value. For example, the HLA-I LOH status may be based on a CNA score. In one or more examples, the HLA-I LOH status may be based on a zygosity score.

In one or more examples, the system can use the HLA-I LOH status to determine a treatment decision for a patient. For example, a positive HLA-I LOH status may be generally correlated with a poor performance of immune checkpoint inhibitors (ICIs). In one or more examples, individuals with a positive HLA-I LOH status may be associated with a poor performance of ICIs, particularly for individuals with a high tumor mutational burden (TMB). Accordingly, if the system determines a positive HLA-I LOH status, the system may forgo recommending ICIs as treatment. Accordingly, if the system determines a positive HLA-I LOH status, the system may forgo recommending ICIs as treatment. Accordingly, the system may recommend a chemotherapeutic agent treatment, a non-ICI targeted treatment, radiation therapy, and/or a hormone therapy. If the system determines a non-positive HLA-I LOH status, the system may recommend ICIs as a treatment option.

In one or more examples, the system can administer a treatment to an individual based on the LOH status of the one or more HLA-I genes. In such examples, if the LOH status of the one or more HLA-I genes is a non-positive LOH status, the treatment may comprise an immune checkpoint inhibitor (ICI) treatment. In such examples, if the LOH status of the one or more HLA-I genes is a positive LOH status, the treatment may comprise a chemotherapeutic agent treatment, a non-ICI targeted treatment, a radiation therapy, a hormone therapy, or a combination thereof.

In one or more examples, the system can assess an immunotherapy resistance of a patient based on the LOH status of the one or more HLA-I genes. In one or more examples, the system can monitor immunotherapy resistance of an individual based on the LOH status of the one or more HLA-I genes over time. For example, samples can be obtained from the individual over a period of time and the system can track the HLA-I LOH status of the individual over the period of time.

In one or more examples, the system can predict one or more clinical outcomes based on the LOH status of the one or more HLA-I genes. In one or more examples, the system can predict an overall survival of the patient based on the LOH status of the one or more HLA-I genes. For example, individuals with a positive HLA-I LOH status may be associated with a lower overall survival than individuals with a non-positive HLA-I LOH status. In one or more examples, the system can predict the progression of a disease. In one or more examples, the system can predict a response of the patient to treatment.

### Methods for Determining a Copy Number Alteration Score

Embodiments of the present disclosure may determine an HLA-I LOH status based on a copy number value. As used herein, a copy number value can correspond to a copy number alteration (CNA) score.

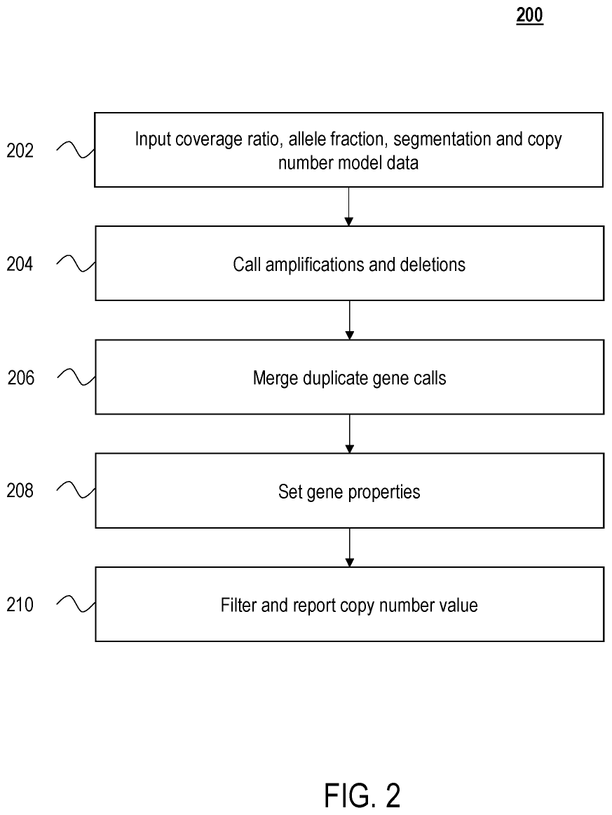

FIG. 2 provides a non-limiting example of a process flowchart for determining a copy number value according to one or more embodiments of this disclosure. In one or more examples, determining the copy number value can be associated with a CNA calling process. In one or more examples, the CNA calling process may be an automated CNA calling process. Additional details regarding process 200 can be found in Provisional Patent Appl. No. 63/253,907, which is incorporated by reference in its entirety herein.

In one or more examples, the process 200 may utilize: (i) a coverage normalization procedure using a “panel of normal” approach that provides proper normalization of chromosome X sequence read data that takes gender into account, (ii) segmentation based on, e.g., a pruned exact linear time (PELT) method customized to use a particular transformation of the coverage ratio data and extended to account for sample contamination, (iii) an iterative sample contamination detection method based on aberrant SNP profiles (determined using a base-substitution noise model and a copy number model profile to identify a contamination signal), (iv) a novel copy number model determination method based on determination of all locally optimal copy number model configurations and prioritization of models (e.g., the copy number model(s) that are most consistent with the sequence read data and are biologically plausible), and/or (v) automated calling of CNA scores based both on the specific copy number model(s) and a scan for additional alterations not explicitly included in the overall copy number model.

As illustrated in FIG. 2, the CNA score calling process begins in step 202 with the input of sequencing coverage ratio data (or “coverage ratio data”), allele fraction data, segmentation data, and copy number model data derived by pre-processing of sequence read data for a plurality of sequence reads that overlap one or more gene loci within one or more subgenomic intervals in the sample to be analyzed (e.g., a patient tumor sample).

In some instances, the coverage ratio data for the sample (e.g., a patient tumor sample) is determined by aligning a plurality of sequence reads that overlap one or more gene loci within one or more subgenomic intervals in the sample and in a control (e.g., a paired normal control, a process-matched control, or a “panel of normal” control) to a reference genome (e.g., an HLA-A allele, an HLA-B allele, and/or an HLA-C allele), and determining a number of sequence reads that overlap each of the one or more gene loci within the one or more subgenomic intervals in the sample and in the control in order to normalize the coverage for the tumor sample to that in the control. In some instances, e.g., if a paired normal control sample is not available, a process-matched control (e.g., a mixture of DNA from a plurality of HapMap cell lines) may be used instead of the paired normal control to normalize coverage. In some instances, e.g., if a paired normal control sample is not available, a “panel of normal” control may be used instead of the paired normal control to normalize coverage.

In some instances, a “panel of normal” (PoN) or “Tangent normalization” control method may be used to normalize sequencing coverage (see, e.g., Tabak, et al. (2019) “The Tangent copy-number inference pipeline for cancer genome analyses”, https://www.biorxiv.org/content/10.1101/566505v1.full.pdf). The Tangent normalization method is a method of normalizing tumor data in order to deal with noise in the data. Specifically, the Tangent method deals with reducing systemic noise resulting from differences in the experimental conditions under which sequencing data from tumors and/or their normal controls were generated. It has been shown that the Tangent normalization method yields a greater reduction in noise than conventional normalization methods.

To begin, let n_{N} be the number of normal non-patient samples (i.e., samples obtained from a plurality of healthy individuals) and n_{T} be the number of tumor samples. Let i be an element of the set {1, 2, . . . , n_{N}} and j be an element of the set {1, 2, . . . , n_{T}}. Define Nᵢ to be the vector of log 2 copy-ratio intensities in genomic order for the i^{th} normal sample. Similarly, define Tⱼ to be the vector of log 2 copy-ratio intensities in genomic order for the j^{th} tumor sample. The normal sample vectors and the tumor sample vectors are elements of the M-dimensional vector space of all possible coverage profiles. Now define a reference subspace N of the vector space of all possible coverage profiles to be the space that contains all linear combinations of the vectors {N₁, N₂, . . . , N_{nN}} of normal samples. N is called the “noise space” and is an (n_{N}−1)-dimensional plane.

Given this setup, the Tangent normalization method proceeds as follows. Start by determining, for each tumor sample vector Tⱼ, the vector in the noise space N that is closest to Tⱼ using a Euclidean metric. Denote this vector p(Tⱼ), the projection of Tⱼ onto N. p(Tⱼ) represents the profile of a normal sample characterized under similar conditions to Tⱼ. The normalization of Tⱼ can now be computed by calculating the difference between Tⱼ and the projection p(Tⱼ) of Tⱼ onto N:

Normalization of Tⱼ=Tⱼ−p(Tⱼ)

The projection p(Tⱼ) can be computed directly using standard linear algebra techniques.

The PoN method uses the observed patterns of systemic noise in the normal samples to remove typical variation. Chromosome X (chrX) has a specific pattern of half the coverage for gene loci on chrX in males since normal males only have one X chromosome. The PoN method thus removes this variation.

In some instances, the allele fraction data for the sample (e.g., a patient tumor sample) is determined by aligning a plurality of sequence reads that overlap one or more gene loci within one or more subgenomic intervals in the sample to a reference genome (e.g., an HLA-A allele, an HLA-B allele, and/or an HLA-C allele), detecting a number of different alleles present at the one or more gene loci in the one or more subgenomic intervals in the sample, and determining an allele fraction for the different alleles present at the one or more gene loci by dividing the number of sequence reads identified for a given allele sequence by the total number of sequence reads identified for the gene locus.

In some instances, the segmentation data for the sample (e.g., a patient tumor sample) is generated by aligning a plurality of sequence reads that overlap one or more gene loci within one or more subgenomic intervals in the sample to a reference genome (e.g., an HLA-A allele, an HLA-B allele, and/or an HLA-C allele), and processing the aligned sequence read data (or other sequencing-related data, e.g., coverage ratio data, allele frequency data, etc., derived from the sequence read data) using a segmentation algorithm (e.g., a circular binary segmentation (CBS) method, a maximum likelihood method, a hidden Markov chain method, a walking Markov method, a Bayesian methods, a long-range correlation method, a change point method, or any combination thereof) to generate a plurality of non-overlapping segments such that the sequence associated with a given segment have the same copy number.

In some instances, segmentation may be performed as part of a copy number modeling process to determine a copy number model that best accounts for the coverage ratio and allele fraction data. For example, in some instances, a copy number model may comprise: a purity estimate (e.g., a fraction of cells in the sample that were derived from a tumor), a segmentation (e.g., a division of the genome into components that have undergone either amplifications or losses), and an assignment of copy number state to each segment, where the copy number state is the number of genomic copies of that segment. In some instances, copy number modeling may be facilitated by transforming haploid coverage ratio data (for example, R_{A} and R_{B}, where R_{A} and R_{B} are the haploid coverage ratios for minor and major alleles A and B, respectively) into sum coverage ratio (R_{A}+R_{B}=(2+ (C_{A}+C_{B})g)/(1+λ g), where C_{A} and C_{B} are the allele counts for the minor and major alleles, A and B, respectively; g=ρ/(1−φ where ρ is the purity; where λ=(Ψ/2), and where Ψ is the ploidy) and difference coverage ratio (R_{A}−R_{B}=((C_{A}−C_{B})g)/(1+λg)) data for major and minor alleles, and plotting the difference coverage ratio data versus the sum coverage ratio data in a plot that may be overlaid with the segment data and a grid representing allowed copy number states.

In some instances, segmentation may be performed in an iterative manner while simultaneously detecting and correcting for sample contamination in the sequence read data. For example, in some instances, the method may comprise estimating a degree of contamination for the sample based on a distribution of minor allele frequencies for a selected set of heterozygous single nucleotide polymorphisms (SNPs). Then, using the estimated degree of contamination as an initial value for a minor allele frequency (MAF) threshold, the sequencing data is iteratively segmented while simultaneously excluding sequencing data from the segmentation process that comprises SNPs having minor allele frequencies that are below the MAF threshold. At each iteration, the remaining SNPs are classified as aberrant (i.e., likely due to contamination) if they have a minor allele frequency that is different from the MAF for other SNPs detected on the same segment, and the MAF threshold is incrementally adjusted based a comparison of the distribution of aberrant SNP minor allele frequencies to the expected distribution of minor allele frequencies for the selected set of heterozygous SNPs. The segmenting, classifying, and MAF threshold adjusting steps are repeated each time the MAF threshold is increased. When no further increase of the MAF threshold is required (or there is no further change in the distribution of aberrant SNP minor allele frequencies, or a specified maximum number of iterations has been reached), the segmentation data and an estimated degree of contamination for the sample (equal to the final value of the MAF threshold) is output. In some instances, the method further comprises using the segmentation data and estimated degree of contamination to build a copy number model that predicts a copy number for one or more gene loci.

In some instances, the segmentation data for the sample (e.g., a patient tumor sample) may be generated using a pruned exact linear time (PELT) method to determine a number of segments required to properly account for the aligned sequence read data (or other sequencing-related data, e.g., coverage ratio data, allele frequency data, etc., derived from the sequence read data), where each segment (and the sequence reads associated with the segment) has the same copy number. In some instances, the segmentation data is generated using a pruned exact linear time (PELT) method that has been customized to use a particular transformation of the coverage ratio and allele fraction data (e.g., a transformation that enables presentation of the coverage ratio and allele fraction data on the same graph while simultaneously overlaying the predicted copy-number states) and extended to account for sample contamination.

In some instances, a copy number model may be used to identify (or predict) the number of copies of each gene locus, the segmentation of the sample, the sample purity, and the sample ploidy (i.e., an average copy number for the sample) that best account for the measured coverage ratio and allele fraction data for the one or more gene loci (i.e., the one or more gene targets). In some instances, the input data used to generate the copy number model also includes coverage ratio and allele fraction data for single nucleotide polymorphisms (SNPs) and introns. The coverage ratio data may be transformed to log 2 coverage ratio data. Examples of copy number modeling methods include, but are not limited to sliding window methods for computing read count in non-overlapping windows, normalized depth-of-coverage and B allele frequency (i.e., the normalized measure of a relative signal intensity ratio for two alleles) methods, circularized binary segmentation (CBS) methods, statistical analyses of mapping density based on mean-shift approaches, hidden Markov models, read depth-based Bayesian information criteria methods, or any combination thereof (see, e.g., Li and Olivier (2013), “Current analysis platforms and methods for detecting copy number variation”, Physiol. Genomics 45 (1): 1-16).

In some instances, the input coverage ratio data or copy number estimates used to generate the copy number model are rounded off to integer values. In some instances the output values reported by the finalized copy number model (e.g., predicted copy number values for segments) are integer values. In some instances, the output values reported by the finalized copy number model (e.g., sample purity, sample ploidy, and copy number values predicted for specific gene loci) are real numbers (i.e., continuous). In some instances, sub-clonal events (e.g., sub-clonal deletion events) may occur which do not fit an integer copy number values and may thus have non-integer predicted copy number values.

In some instances, the copy number model may determine that the sample purity (or tumor fraction) has a value ranging from 0.05 to 1.0. In some instances, the determined sample purity may be at least 0.05, at least 0.1, at least 0.2, at least 0.3, at least 0.4, at least 0.5, at least 0.6, at least 0.7, at least 0.8, at least 0.9, at least 0.95, at least 0.98, or at least 0.99. In some instances, the determined sample purity may be at most 0.99, at most 0.98, at most 0.95, at most 0.9, at most 0.8, at most 0.7, at most 0.6, at most 0.5, at most 0.4, at most 0.3, at most 0.2, at most 0.1, or at most 0.05. Any of the lower and upper values described in this paragraph may be combined to form a range included within the present disclosure, for example, in some instances, the determined sample purity may range from 0.1 to 0.8. Those of skill in the art will recognize that the determined sample purity in a given instance may have any value within this range, e.g., about 0.64.

In some instances, the copy number model may determine that the sample ploidy has a value ranging from 1.0 to 10.0. In some instances, the determined sample ploidy may be at least 1.0, at least 2.0, at least 3.0, at least 4.0, at least 5.0, at least 6.0, at least 7.0, at least 8.0, at least 9.0, or at least 10.0. In some instances, the determined sample ploidy may be at most 10.0, at most 9.0, at most 8.0, at most 7.0, at most 6.0, at most 5.0, at most 4.0, at most 3.0, at most 2.0, or at most 1.0. Any of the lower and upper values described in this paragraph may be combined to form a range included within the present disclosure, for example, in some instances, the determined sample ploidy may range from 1.0 to 8.0. Those of skill in the art will recognize that the determined sample ploidy in a given instance may have any value within this range, e.g., about 3.4. In some instances, the sample ploidy may be rounded off and reported as an integer value.

In some instances, the copy number model may predict a copy number for a given gene locus (or segment with which it is associated) ranging from 0 to 500. In some instances, the predicted copy number is at least 0, at least 2, at least 4, at least 6, at least 8, at least 10, at least 20, at least 40, at least 60, at least 80, at least 100, at least 200, at least 300, at least 400, or at least 500. In some instances, the predicted copy number is at most 500, at most 4400, at most 300, at most 200, at most 100, at most 80, at most 60, at most 40, at most 20, at most 10, at most 8, at most 6, at most 4, at most 2, or at most 0. Any of the lower and upper values described in this paragraph may be combined to form a range included within the present disclosure, for example, in some instances, the predicted copy number may range from 1 to 100. Those of skill in the art will recognize that the predicted copy number may have any value within this range, e.g., 7. In some instances, the predicted copy number for a gene locus may be a real valued number rather than an integer.

Referring again to FIG. 2, at step 204, amplifications (e.g., increases in the number of copies of a gene locus) or deletions (e.g., deletions of a complete or partial gene locus) of each gene locus of the one or more loci being analyzed are determined on a segment by segment basis.

At step 206 of FIG. 2, duplicate gene calls, or more formally, duplicate calls for “gene objects” (i.e., digital data constructs that hold a set of properties (e.g., sequence location, target allele sequences, coverage ratios, etc.) associated with a given gene locus) are merged. Duplicate calls may arise, for example, if a gene sequence is broken into two sub-sequences, and both sub-sequences are called as gene loci comprising amplifications or deletions, thus generating more than one gene object for the locus. In other instances, deletions may be called using both a copy number prediction that comes directly from the copy-number model data and by a partial deletion scanning method (e.g., a method that looks for sequence read(s) that overlap but deviate significantly from the target allele sequence(s) and results in a partial deletion call), in which case more than one gene object is again generated for the locus.

At step 208 in FIG. 2, the set of properties associated with each gene locus (or gene object) is updated.

At step 210 of FIG. 2, the CNA score results are filtered to determine the CNA score. For example, the system can perform a quality control (QC) procedure for assessing the quality of the sequence read data, the sample purity (e.g., by comparison of a sample purity to a specified sample purity threshold), successful convergence of the copy number model, and/or to assess the reliability of CNA score calls for individual gene loci, etc., and prepared for reporting.

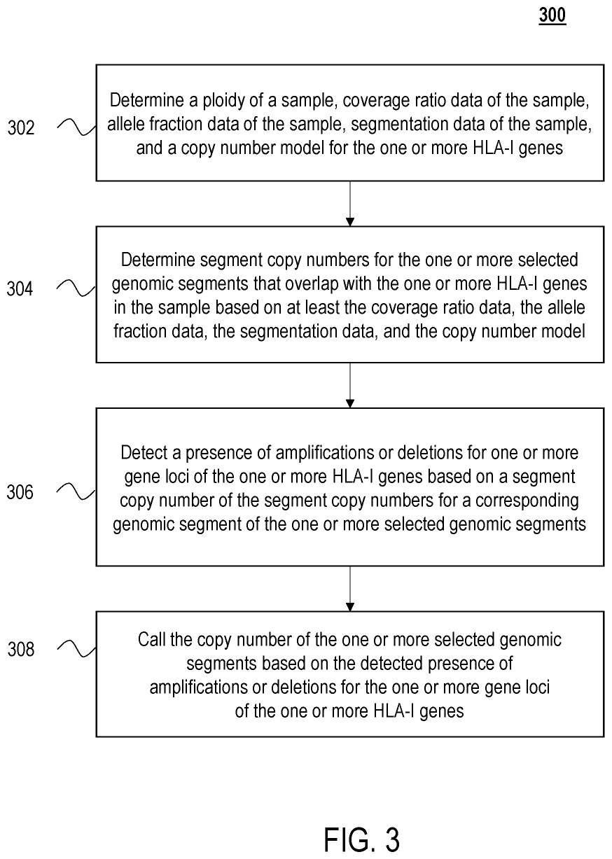

FIG. 3 provides a non-limiting example of a process flowchart 300 for determining a copy number value in accordance with one or more embodiments of the present disclosure. Process 300 can be performed, for example, using one or more electronic devices implementing a software platform. In some examples, process 300 is performed using a client-server system, and the blocks of process 300 are divided up in any manner between the server and a client device. In other examples, the blocks of process 300 are divided up between the server and multiple client devices. Thus, while portions of process 300 are described herein as being performed by particular devices of a client-server system, it will be appreciated that process 300 is not so limited. In other examples, process 300 is performed using only a client device or only multiple client devices. In process 300, some blocks are, optionally, combined, the order of some blocks is, optionally, changed, and some blocks are, optionally, omitted. In some examples, additional steps may be performed in combination with the process 300. Accordingly, the operations as illustrated (and described in greater detail below) are exemplary by nature and, as such, should not be viewed as limiting.

At step 302 in FIG. 3, the system can determine a ploidy of a sample, coverage ratio data of the sample, allele fraction data of the sample, segmentation data of the sample, and a copy number model for the one or more HLA-I genes. In one or more examples, step 302 can correspond to one or more aspects of step 202 of FIG. 2 described above.

At step 304 in FIG. 3, the system can determine segment copy numbers for the one or more selected genomic segments that overlap with the one or more HLA-I genes in the sample based on at least the coverage ratio data, the allele fraction data, the segmentation data, and the copy number model. In one or more examples, step 304 can correspond to one or more aspects of step 202 of FIG. 2 described above.

For example, the system can determine the coverage ratio data in by aligning a plurality of sequence reads of the one or more selected genomic segments that overlap with the one or more HLA-I genes in the sample to a reference genome. The system can then align a plurality of sequence reads of one or more selected genomic segments that overlap with the one or more HLA-I genes in a control sample to the reference genome. The system can then determine a number of sequence reads that overlap each of the one or more gene loci of the one or more HLA-I genes within the one or more selected genomic segments in the sample. The system can then determine a number of sequence reads that overlap each of the one or more gene loci of the one or more HLA-I genes within the one or more selected genomic segments in the control sample. The coverage ratio data may be based on the number of sequence reads that overlap each of the one or more gene loci of the one or more HLA-I genes within the one or more selected genomic segments in the control sample.

At step 306 in FIG. 3, the system can detect a presence of amplifications or deletions for one or more gene loci of the one or more HLA-I genes based on a segment copy number of the segment copy numbers for a corresponding genomic segment of the one or more selected genomic segments. In one or more examples, step 306 can correspond to one or more aspects of step 204 of FIG. 2 described above.

At step 308 in FIG. 3, the system can call the copy number value of the one or more selected genomic segments based on the detected presence of amplifications or deletions for the one or more gene loci of the one or more HLA-I genes. In one or more examples, the copy number value of the one or more selected genomic segments may be based on the whether neither an amplification nor a deletion is detected. In such embodiments, the system may determine a non-positive (e.g., negative) HLA-I LOH status or a positive HLA-I LOH status for a neutral LOH. In one or more examples, step 306 can correspond to one or more aspects of step 210 of FIG. 2 described above.

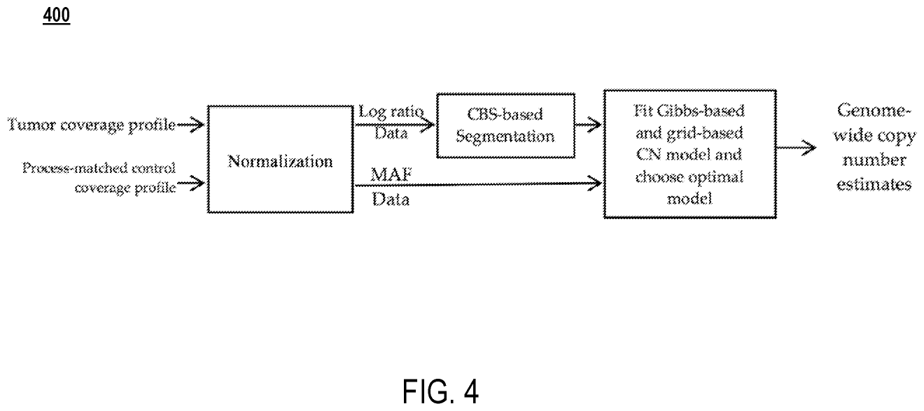

FIG. 4 provides a non-limiting example of a process flowchart 400 for determining a copy number value in accordance with embodiments of the present disclosure. Process 400 can be performed, for example, using one or more electronic devices implementing a software platform. In some examples, process 400 is performed using a client-server system, and the blocks of process 400 are divided up in any manner between the server and a client device. In other examples, the blocks of process 400 are divided up between the server and multiple client devices. Thus, while portions of process 400 are described herein as being performed by particular devices of a client-server system, it will be appreciated that process 400 is not so limited. In other examples, process 400 is performed using only a client device or only multiple client devices. In process 400, some blocks are, optionally, combined, the order of some blocks is, optionally, changed, and some blocks are, optionally, omitted. In some examples, additional steps may be performed in combination with the process 400. Accordingly, the operations as illustrated (and described in greater detail below) are exemplary by nature and, as such, should not be viewed as limiting.

As shown in FIG. 4, to determine the copy number value (e.g., CNA score) aligned DNA sequences of the tumor specimen can be normalized against a process-matched normal coverage profile data. Based on the normalization, the system can produce log-ratio data and minor allele frequency (MAF) data. Next, whole-genome segmentation can be performed using a circular binary segmentation (CBS) algorithm on the log-ratio data. Then, a Gibbs sampler fitted copy number model and a grid-based model are fit to the segmented log-ratio and MAF data, producing genome-wide copy number estimates. Finally, the degree of fit of candidate models returned by Gibbs sampling and grid sampling are compared and the optimal model may be selected. In one or more examples, an automated heuristic may be used to select the optimal model. In one or more examples, the genome segmentation can comprise one or more of a circular binary segmentation algorithm, an HMM based method, a Wavelett based method, or a Cluster along Chromosomes method. In one or more examples, the process 400 can further include segmenting a genomic sequence of the one or more selected genomic sequences that overlap with the one or more HLA-I genes in the sample into partial genomic segments of equal copy number. See Sun, et al. (2015) “A computational approach to distinguish somatic vs. germline origin of genomic alterations from deep sequencing of cancer specimens without a matched normal”.

### Methods for Determining a Zygosity Score

Embodiments of the present disclosure may determine an HLA-I LOH status based on a copy number value. As used herein, a copy number value can correspond to a zygosity score.

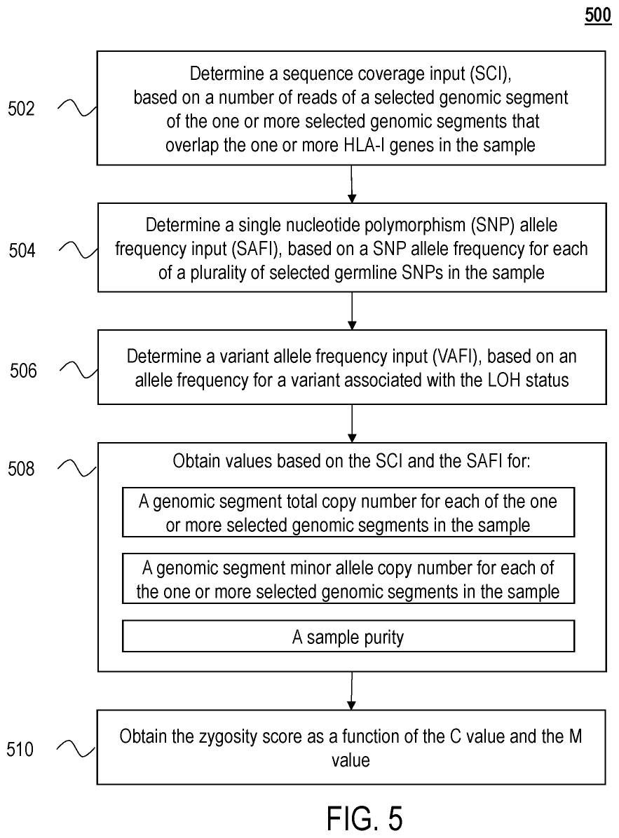

FIG. 5 provides a non-limiting example of a process flowchart 500 for determining a copy number value according to one or more embodiments of this disclosure. In one or more examples, determining the copy number value can be based on a zygosity score. See U.S. Pat. No. 9,792,403.

Process 500 can be performed, for example, using one or more electronic devices implementing a software platform. In some examples, process 500 is performed using a client-server system, and the blocks of process 500 are divided up in any manner between the server and a client device. In other examples, the blocks of process 500 are divided up between the server and multiple client devices. Thus, while portions of process 500 are described herein as being performed by particular devices of a client-server system, it will be appreciated that process 500 is not so limited. In other examples, process 500 is performed using only a client device or only multiple client devices. In process 500, some blocks are, optionally, combined, the order of some blocks is, optionally, changed, and some blocks are, optionally, omitted. In some examples, additional steps may be performed in combination with the process 500. Accordingly, the operations as illustrated (and described in greater detail below) are exemplary by nature and, as such, should not be viewed as limiting.

At step 502 in FIG. 5, the system can determine a sequence coverage input (SCI), based on a number of reads of a selected genomic segment of the one or more selected genomic segments that overlap the one or more HLA-I genes in the sample. In one or more examples, the system can assign SCI values to the selected genomic segments (e.g., a plurality of subgenomic intervals).

In one or more examples, system may be configured to capture data on a genomic sequence coverage for specified subgenomic intervals. The system can define a variable for the SCI based on the values for sequence coverage at the specified subgenomic intervals. In one or more examples, the system includes a user interface display configured to accept user input to define the specified subgenomic intervals. In other embodiments, the subgenomic intervals can be pre-defined as part of genetic testing and/or analysis. Further, the system can also be configured to identify the subgenomic intervals to analyze automatically (e.g., based on segmentation analysis, etc.). Once the subgenomic intervals are specified, the system can capture a value for sequence coverage for each of a plurality of specified subgenomic intervals. The captured values can be normalized, averaged, or weighted to prevent outlier values from skewing subsequent calculations.

In one or more examples, the system and/or system components are configured to fit the genome-wide copy number model to the SCI using Equation 1:

$$log {Ratio}_{i} = {log}_{2} \frac{{pC}_{i} + 2 ( 1 - p )}{p ψ + 2 ( 1 - p )} \qquad ( 1 )$$

where ψ is tumor ploidy, C is a copy number, and p is a sample purity. The system and/or system components can calculate ψ as =(ΣiliCi)/Σili, where li is the length of a genomic segment.

At step 504 in FIG. 5, the system can determine a single nucleotide polymorphism (SNP) allele frequency input (SAFI), based on a SNP allele frequency for each of a plurality of selected germline SNPs in the sample.

In one or more examples, the system can be configured to derive an allele frequency value according to specification of germline SNPs in the tumor sample. The system can define a variable for a SAFI based on the values for allele frequency for the selected germline SNPs. In some embodiments, the system specifies the germline SNPs on which to capture values for allele frequency (e.g., based on pre-specified selection, automatically based on analysis of the tumor sample, etc.). In other embodiments, the user interface can also be configured to accept selection of germline SNPs within genetic sequencing information obtained on, for example, a tumor sample.

The system can also be configured to fit the genome-wide copy number model to the SAFI using Equation 2:

$$AF = \frac{pM + 1 ( 1 - p )}{pC + 2 ( 1 - p )} \qquad ( 2 )$$

where AF is allele frequency. Various fitting methodologies can be executed by the system to determine a g value indicative of a germline or somatic origin of the sample (e.g., Markov chain Monte Carlo (MCMC) algorithm, e.g., ASCAT (Allele-Specific Copy Number Analysis of Tumors), OncoSNP, or PICNIC (Predicting Integral Copy Numbers In Cancer).

At step 506 in FIG. 5, the system can determine a variant allele frequency input (VAFI), based on an allele frequency for a variant associated with the LOH status.

For example, the system can be configured to capture and/or calculate additional values from genetic sequence information (including, e.g., captured from testing systems and/or components or generated by the characterization system directly). In one or more examples, the system can capture the VAFI for a given variant (e.g., a mutation) from testing data. In another example, the system can generate the data for capturing the allele frequency responsive to genetic sequence testing performed on the sample.

At step 508 in FIG. 5, the system can obtain values based on the SCI and the SAFI for each of: a genomic segment total copy number (C value) for each of the one or more selected a plurality of genomic segments; a genomic segment minor allele copy number (M value) for each of the one or more selected genomic segments in the sample; and a sample purity (p).

At step 510 in FIG. 5, the system can obtain the zygosity score as a function of the C value and the M value. For example, a value of M equal to 0 not equal to C is indicative of absence of the variant, a non-zero value of M equal to C is indicative of homozygosity of the variant (e.g., LOH), a value of M equal to 0 equal to C is indicative of a homozygous deletion of the variant, and a non-zero value of M not equal to C is indicative of heterozygosity of the variant. Based on the zygosity score a characterization model can be generated for a variant zygosity.

In further examples, the system can calculate a value indicative of the zygosity of the variant in the sample as a function of the acquired and/or calculated C value and M value. For example, a M value equal to 0 not equal to a C value is indicative of absence of the variant, a non-zero M value equal to a C value is indicative of homozygosity of the variant (e.g., LOH), a M value equal to 0 equal to a C value is indicative of homozygous deletion of the variant, and a non-zero value of M not equal to a C value is indicative of heterozygosity of the variant.

In some implementations, the system can also be configured to determine a confidence level associated with any calculation and/or calculated value (e.g., based on statistical analysis of the input(s) and computational values used to derive an output). The system can use determinations on the confidence of calculations and/or calculated values in interpreting classification outputs. In one example, the not-determinable range of values can be increased where the degree of confidence associated with the calculation of the g value is low. In another example, the not-determinable range of values can be decreased where the degree of confidence associated with the calculation of the g value is high.

### Methods for Determining a Loss of Heterozygosity of HLA-I Genes Using Allelic Imbalance Data

One or more embodiments of the present disclosure provide methods for determining HLA-I loss of heterozygosity (LOH) of a sample. One or more embodiments of this disclosure provide methods for identifying HLA-I LOH when allelic imbalance information is available. One or more embodiments of this disclosure provide methods for identifying HLA-I LOH when allelic imbalance information is available, but copy number variation information is unavailable.

In one or more examples, process 600 can be applied to at least one of a tissue biopsy sample, a liquid biopsy sample, or a normal control. In one or more examples, process 600 can be applied to liquid biopsy samples, where copy number information may be unavailable or unreliable.

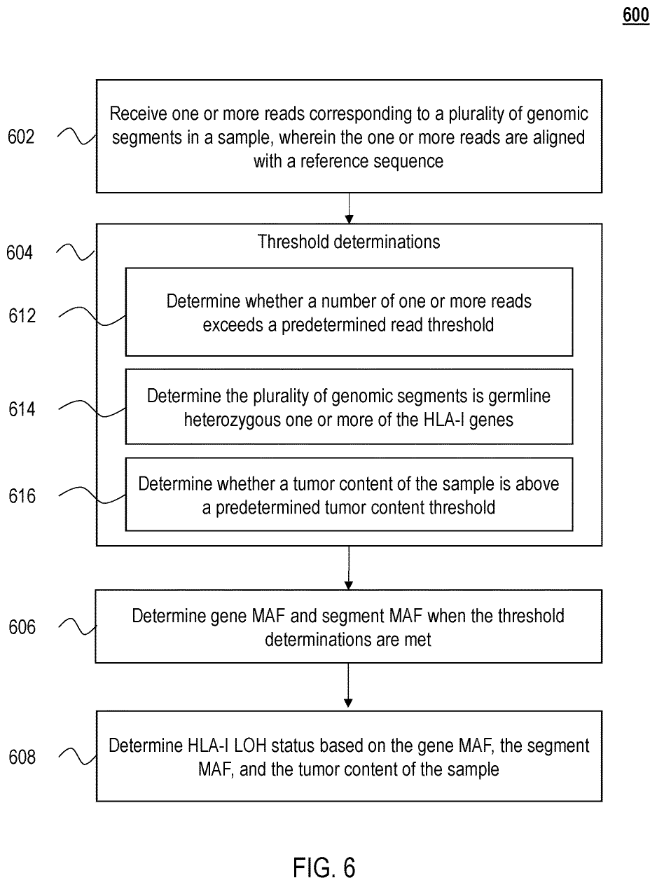

FIG. 6 provides a non-limiting example of a process 600 for determining an HLA-I LOH status of a sample. In one or more examples, the HLA-I LOH status may be based on one or more HLA-I genes, for example, an HLA-A gene, an HLA-B gene, and/or an HLA-C gene. In one or more examples, process 600 determines the HLA-I LOH status of the sample based on allelic imbalance information without relying on copy number information of the sample.

Process 600 can be performed, for example, using one or more electronic devices implementing a software platform. In some examples, process 600 is performed using a client-server system, and the blocks of process 600 are divided up in any manner between the server and a client device. In other examples, the blocks of process 600 are divided up between the server and multiple client devices. Thus, while portions of process 600 are described herein as being performed by particular devices of a client-server system, it will be appreciated that process 600 is not so limited. In other examples, process 600 is performed using only a client device or only multiple client devices. In process 600, some blocks are, optionally, combined, the order of some blocks is, optionally, changed, and some blocks are, optionally, omitted. In some examples, additional steps may be performed in combination with the process 600. Accordingly, the operations as illustrated (and described in greater detail below) are exemplary by nature and, as such, should not be viewed as limiting.

At step 602 in FIG. 6, the system can receive one or more reads corresponding to a plurality of genomic segments in a sample, wherein the one or more reads are aligned with a reference sequence. In one or more examples, the reference sequence can correspond to one or more HLA-I gene alleles (e.g., an HLA-A allele, an HLA-B allele, and/or an HLA-C allele). In one or more examples, the system can sequence the one or more reads corresponding to the plurality of genomic segments in the sample. In one or more examples, the genomic segments may be baited for one or more HLA-I genes (e.g., an HLA-A allele, an HLA-B allele, and/or an HLA-C allele).

In one or more examples, the system can genotype the plurality of genomic segments in the sample. In such examples, the system can obtain a reference sequence and align the one or more reads corresponding to a plurality of genomic segments in the sample to the reference sequence.

At step 604 in FIG. 6, the system can perform one or more threshold determinations. For example, the system can determine: whether a number of one or more reads exceeds a predetermined read threshold (read determination) at step 612, the plurality of genomic segments is germline heterozygous one or more of the HLA-I genes (heterozygosity determination) at step 614, and whether a tumor content of the sample is above a predetermined tumor content threshold (tumor content determination) at 616.

At step 612 in FIG. 6, the system can determine whether a number of one or more reads exceeds a predetermined read threshold. For example, the system may align the one or more reads corresponding to the plurality of genomic segments in the sample to a reference sequence, where the reference sequence comprises one or more reads of an HLA allele of the one or more HLA-I genes (e.g., an HLA-A allele, an HLA-B allele, and/or an HLA-C allele). The system may then determine a number of the one or more reads corresponding to the plurality of genomic segments that are aligned with the reference sequence and compare the number of the aligned reads to a predetermined read threshold. In one or more examples, the read threshold may be is one of 1000 reads, 1100 reads, 1200 reads, 1300 reads, 1400 reads, 1500 reads, 1600 reads, 1700 reads, 1800 reads, 1900 reads, 2000 reads, 2100 reads, 2200 reads, 2300 reads, 2400 reads, 2500 reads, 2600 reads, 2700 reads, 2800 reads, 2900 reads, or 3000 reads.

As an example, the system could align the one or more reads of a first genomic segment in the sample to a reference sequence associated with an HLA-A allele. The system could then determine the number of reads in the first genomic segment that are aligned with the HLA-A reference sequence and compare this number with the predetermined read threshold. If the number of reads is exceeds the predetermined threshold, e.g., if the number of reads corresponds to 1005 and the read threshold is 1000, then the system may determine that the read threshold is met. In one or more examples, the read determination may be made on a gene by gene basis, e.g., a separate read determination may be performed for each of an HLA-A allele, an HLA-B allele, and/or an HLA-C allele.

At step 614 in FIG. 6, the system can determine the plurality of genomic segments is germline heterozygous to one or more of the HLA-I genes. In one or more examples, determining whether the plurality of genomic segments in the sample are germline heterozygous can be based on an allele fraction of the sample. In one or more examples, if the allele fraction of the sample with respect to one or more of the HLA-I genes is about 0.5, the then sample may be determined to be germline heterozygous. In one or more examples, if the allele fraction of the sample is about 0.25-0.75, then the sample may be determined to be germline heterozygous.

For example, the system may determine whether the sample is germline heterozygous at the HLA-A gene. In such embodiments, the system can obtain an allele fraction of the sample at the HLA-A gene. If the allele fraction of the sample at the HLA-A gene is about 0.5, then the system may determine that the sample is heterozygous at the HLA-A gene and that the heterozygosity threshold is met. In one or more examples, the heterozygosity determination may be made on a gene by gene basis, e.g., a heterozygosity determination may be performed for each of an HLA-A gene, an HLA-B gene, and an HLA-C gene.

At step 616 in FIG. 6, the system can determine whether a tumor content of the sample is above a predetermined tumor content threshold. In one or more examples, the tumor content can be based on a tumor purity. In one or more examples, the tumor content can be based on a tumor fraction. In one or more embodiments, the tumor content can be based on a maximum somatic allele frequency (MSAF). In one or more examples, the tumor content is a value indicative of a ratio of circulating tumor DNA (ctDNA) to total cell-free DNA (cfDNA) in the sample. In one or more examples the tumor content threshold can correspond to a tumor content of 5%. In one or more examples, the tumor content threshold can correspond to an amount between 1% and 10%. In order for the sample to be properly processed, the sample should include an amount of non-tumor content. For example, if the sample did not include non-tumor content, the system may not be able to determine whether any observed homozygosity is due to somatic LOH or due to germline homozygosity. The tumor content determination may be made on a sample basis, e.g., not a gene by gene basis. Methods for determining tumor content in accordance with embodiments of the present disclosure will be described in greater detail below.

In one or more examples, the threshold determinations can be performed in sequence. In one or more examples, two or more of the threshold determinations may be made in parallel. A skilled artisan will understand the order of performing the threshold determinations is not intended to limit the scope of this disclosure.

In one or more examples, if one or more of the read determination, heterozygosity determination, and tumor content determination is met, then the system may move to the next step 606 to further process the sample. In such examples, if none of the threshold determinations are met, then the sample is discarded. In one or more examples, if two or more of the read determination, heterozygosity determination, and tumor content determination are met, then the system may move to the next step 606 to further process the sample. In such examples, if zero or one of the threshold determinations are met, then the sample is discarded, e.g., no HLA-I LOH determination is made. In one or more examples, if the determination relates to a specific gene, then the threshold determinations should be met for the same gene. For example, if the read determination of a sample is met for both an HLA-A allele and an HLA-B allele, and the heterozygosity determination of the sample is met for the HLA-A gene and not the HLA-B gene, the system may proceed to the next step of process 600 with respect to the HLA-A gene to determine whether the sample has an HLA-A LOH. But the system may not proceed to the next step of process 600 with respect to the HLA-B gene because two or more threshold determinations were not met.

In one or more examples, in order for the system to move to the next step of the process 600, the heterozygosity determination, and tumor content determination should each be met. In such examples, two or less of the threshold determinations are met, then the sample is discarded. Embodiments where all the read determination, heterozygosity determination, and tumor content determination are met may provide more robust samples for determining the HLA-I LOH status. In one or more examples, if the determination relates to a specific gene, then the threshold determinations should be met for the same gene, as described above.

At step 606 in FIG. 6, if the system determines the plurality of genomic segments are germline heterozygous, the number of reads in the one or more reads exceeds the predetermined read threshold, and/or the tumor content is above the predetermined tumor content threshold, the system can then determine a gene MAF and a segment MAF. In one more examples, the system can obtain the gene MAF and segment MAF based on one or more analyses performed on the sample.

At step 608 in FIG. 6, the system can determine an HLA-I LOH status based on the gene MAF, the segment MAF, and the tumor content of the sample. In one or more examples, the HLA-I LOH status can be determined based on the values shown in Table I provided below. For example, if the MSAF of the sample is between 1-5%, the HLA-I gene MAF is less than 46% and the HLA-I segment MAF is less than 35%, then the sample may be determined to have a HLA-I LOH positive status. In one or more examples, if the MSAF of the sample is between 6-9%, the HLA-I gene MAF is less than 46% and the HLA-I segment MAF is less than 33%, then the sample may be determined to have a HLA-I LOH positive status. In one or more examples, if the MSAF of the sample is between 10-20%, the HLA-I gene MAF is less than 43% and the HLA-I segment MAF is less than 33%, then the sample may be determined to have a HLA-I LOH positive status.

TABLE I

| | | HLA-I Segment |
|---|---|---|
| MSAF | HLA-I Gene MAF | MAF |
| 1-5% | <46% | <35% |
| 6-9% | <46% | <33% |
| 10-20% | <43% | <33% |

As shown in Table I, as the MSAF increases, the HLA-I status can be determined with a smaller amount of HLA-I gene MAF and HLA-I segment MAF because there is a greater amount of tumor content in the sample.

In one or more examples, a model can be trained to determine the read and the tumor content thresholds discussed above. In one or more examples, training may include the system receiving, one or more reads corresponding to one or more selected genomic segments in a paired sample, wherein the one or more reads corresponding to the one or more selected genomic segments are aligned with the reference sequence. Each paired sample can correspond to a first solid sample and a second liquid sample from an individual. For each paired sample, the system can fit one or more values associated with the one or more reads corresponding to the one or more selected genomic segments to a model. For each paired sample, the system can also determine a LOH status of the corresponding paired sample based on the fitted model. The system can then determine the predetermined read threshold and the tumor content threshold based on the fitted model and the LOH status for the one or more selected genomic segments in the paired sample. For example, the system may select the thresholds to accurately predict the HLA-I LOH status for a majority of the paired-samples. In one or more examples, these training methods can be used to determine thresholds for MSAF, HLA-I gene MAF, and HLA-I segment MAF.

In one or more examples, the system can use the LOH status to determine a treatment decision for a patient. For example, a positive HLA-I LOH status may be generally correlated with a poor performance of immune checkpoint inhibitors (ICIs). Accordingly, if the system determines a positive HLA-I LOH status, the system may forgo recommending ICIs as treatment. Accordingly, the system may recommend a chemotherapeutic agent treatment, a non-ICI targeted treatment, radiation therapy, and/or a hormone therapy. If the system determines a non-positive HLA-I LOH status, the system may recommend ICIs as a treatment option.

In one or more examples, the system can administer a treatment to an individual based on the LOH status of the one or more HLA-I genes. In such examples, if the LOH status of the one or more HLA-I genes is a non-positive LOH status, the treatment may comprise an immune checkpoint inhibitor (ICI) treatment. In such examples, if the LOH status of the one or more HLA-I genes is a positive LOH status, the treatment may comprise a chemotherapeutic agent treatment, a non-ICI targeted treatment, a radiation therapy, a hormone therapy, or a combination thereof.

In one or more examples, the system can assess an immunotherapy resistance of a patient based on the LOH status of the one or more HLA-I genes. In one or more examples, the system can monitor immunotherapy resistance of an individual based on the LOH status of the one or more HLA-I genes over time. For example, samples can be obtained from the individual over a period of time and the system can track the HLA-I LOH status of the individual over the period of time.

In one or more examples, the system can predict one or more clinical outcomes based on the LOH status of the one or more HLA-I genes. In one or more examples, the system can predict an overall survival of the patient based on the LOH status of the one or more HLA-I genes.

### Determining Tumor Content

Embodiments of the present disclosure may use any suitable technique to whether the tumor content of the sample is above a predetermined tumor content threshold. As noted above, the tumor content can include a tumor purity, tumor fraction, and/or MSAF. The methods for determining a tumor content of the sample is not intended to limit the scope of this disclosure.

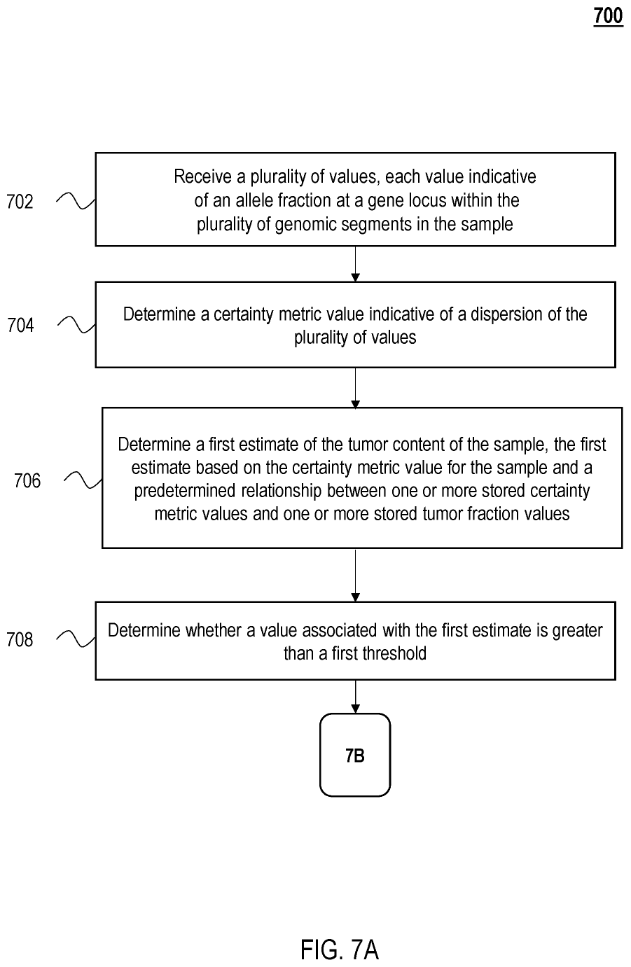

FIG. 7A and FIG. 7B show process 700 for determining a tumor content in accordance with embodiments of the present disclosure. Process 700 can be performed, for example, using one or more electronic devices implementing a software platform. In some examples, process 700 is performed using a client-server system, and the blocks of process 700 are divided up in any manner between the server and a client device. In other examples, the blocks of process 700 are divided up between the server and multiple client devices. Thus, while portions of process 700 are described herein as being performed by particular devices of a client-server system, it will be appreciated that process 700 is not so limited. In other examples, process 700 is performed using only a client device or only multiple client devices. In process 700, some blocks are, optionally, combined, the order of some blocks is, optionally, changed, and some blocks are, optionally, omitted. In some examples, additional steps may be performed in combination with the process 700. Accordingly, the operations as illustrated (and described in greater detail below) are exemplary by nature and, as such, should not be viewed as limiting.

At step 702 in FIG. 7A, the system can receive a plurality of values, where each value is indicative of an allele fraction at a gene locus within the plurality of genomic segments in the sample. In one or more examples, the gene locus can correspond to one or more HLA-I genes (e.g., an HLA-A gene, an HLA-B gene, an HLA-C gene, etc.). In one or more examples, the gene locus may correspond to the one or more HLA-I genes that meet the requisite threshold determinations at step 604 in FIG. 6.

At step 704 in FIG. 7A, the system can determine a certainty metric value indicative of a dispersion of the plurality of values. Tumor content can be associated with allele fraction dispersion across a plurality of analyzed loci. The term “certainty metric,” as used herein, may refer to a metric derived from a measure or value of a target variable. In some embodiments, the target variable may represent an abundance of a subgenomic interval, or an allele associated with the subgenomic interval, in a sample. In some examples, the certainty metric may be a deviation of an allele fraction from an expected allele fraction. In other examples, the certainty metric may be a measure of allele intensity. These examples are intended to be illustrative, and other certainty metrics may be used.

At step 706 in FIG. 7A, the system can determine a first estimate of the tumor content of the sample, the first estimate based on the certainty metric value for the sample and a predetermined relationship between one or more stored certainty metric values and one or more stored tumor fraction values. At step 708 in FIG. 7A, the system can determine whether a value associated with the first estimate is greater than a first threshold. In one or more examples, the first threshold may be, a limit-of-detection (LoD) or specified confidence level for determining a tumor content.

At step 710 in FIG. 7B, the system can determine whether the value associated with the first estimate is greater than a first threshold. If the first estimate of the tumor content of the sample is greater than the first threshold, the system can output the first estimate of the tumor content as the tumor content of the sample at step 712. If the first estimate of the tumor content of the sample is not greater than the first threshold, the system can determine a second estimate of the tumor content of the sample at step 714. At step 716, the system can output the second estimate as the tumor content of the sample.

In one or more examples, the second estimate of the tumor content can be determined based on the quality metric. For example, the system can determine whether a quality metric for the plurality of values is greater than a second threshold. Based on a determination that the quality metric for the plurality of values is greater than the second threshold, the system can determine the second estimate for the tumor content of the sample based on a first determination of somatic allele frequency. Based on a determination that the quality metric for the plurality of values is less than or equal to the second threshold, the system can determine the second estimate for the tumor content of the sample based on a second determination of somatic allele frequency.

Examples of a quality metric that may be used as a quality metric and/or used to calculate a quality metric for the sequencing data include, but are not limited to, an average sequence coverage for the sample, a minimum average sequence coverage for the sample, an allele coverage at each of the corresponding loci in the plurality of loci, a minimum allele coverage at each of the corresponding loci in the plurality of loci, a degree of nucleic acid contamination in the sample (determined, e.g., by quantifying aberrations in SNP allele frequencies), a maximum degree of nucleic acid contamination in the sample, a number of single nucleotide polymorphism (SNP) loci within the plurality of loci examined a minimum number of single nucleotide polymorphism (SNP) loci within the plurality of loci examined, or any combination thereof. In some instances, the quality threshold (or second threshold) may comprise a specified lower limit of the quality metric.

At step 718 in FIG. 7B, the system can compare the tumor content of the sample to the tumor content threshold. In one or more examples, step 718 can correspond to step 616 in FIG. 6 described above.

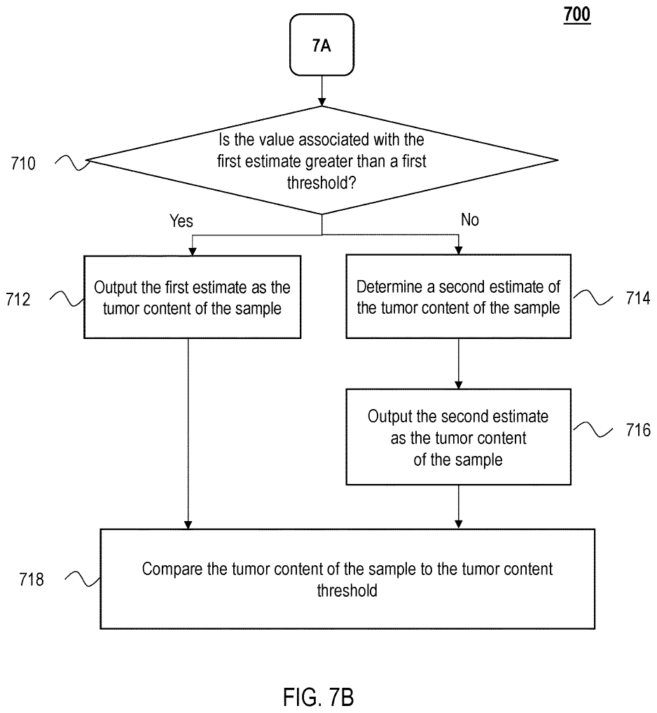

FIG. 8 shows a process 800 of estimating a tumor content of a sample according to embodiments of this disclosure. In one or more examples process 800 can correspond to a method of estimating a tumor fraction from a sample. The process 800 begins at step 802. At step 804, a value for a target variable associated with a subgenomic interval is obtained, e.g., directly obtained, from a sample from a subject. The target variable may be, for example, an allele fraction. The sample may be, e.g., a liquid sample or a solid sample.

In some examples, a patient allele fraction for at least one heterozygous single nucleotide polymorphism (SNP) site is determined from a biopsy taken from a patient. In one example, the biopsy may be a liquid biopsy, i.e., a sample of non-solid biological tissue, for example, blood. The disclosure is not so limited, however, and is intended to cover any solid or liquid assays or biopsies without limitation. In an embodiment, the liquid biopsy comprises a blood sample. In an embodiment, the liquid biopsy comprises cell free DNA (cfDNA). In an embodiment, the liquid biopsy comprises circulating tumor DNA (ctDNA). In an embodiment, the liquid biopsy comprises DNA shed from a tumor. In an embodiment, the liquid biopsy comprises nucleic acids other than DNA, e.g., RNA. In an embodiment, the liquid biopsy comprises circulating tumor cells (CTCs). Other types of liquid biopsies are described, e.g., in Crowley et al. Nat Rev Clin Oncol. 2013; 10 (8): 472-484, the content of which is incorporated by reference in its entirety.

At step 806, a certainty metric may be determined from the target variable. At step 808, a determined relationship is accessed between a stored certainty metric and a stored tumor fraction. The determined relationship may include historical sample data (collected from patients or other test subjects) relating a certainty metric (e.g., a sampled allele fraction deviation) for at least one heterozygous SNP site to a corresponding sampled tumor fraction. In some examples, the sampled allele coverage deviation is a “noise” metric, reflecting the degree to which an allele fraction varies from an expected value. In some examples, the number of data points correlating tumor fraction to noise metrics calculated from the allele fraction may exceed one hundred (100), one thousand (1,000), ten thousand (10,000), or more.

In one example, the determined relationship may be derived from an in silico process, and the analysis may be performed by a machine learning process. The process may perform a sample dilution (e.g., using a matched normal) starting at a particular tumor fraction in order to correlate one or more coverage deviation metrics (e.g., allele fraction values) across one or more subgenomic intervals (e.g., SNPs, SNP bins, and/or chromosomes). The metric may be a measure of the frequency and degree to which tumor fraction falls in between the values of 0 or 1. Averaged “noise” metrics between 0 and 1 (exclusive) may be correlated with an expected or estimated tumor fraction. In some embodiments, the disclosed methods may comprise: obtaining a training dataset comprising a plurality of relationships between a plurality of training certainty metric values and associated training tumor fraction values; training a machine learning model based on the training dataset; and using the trained machine learning model to determine a tumor fraction value from the certainty metric value for the sample.

The number of elements associated with subgenomic intervals that contribute to the determination of the certainty metric value, which is correlated to tumor fraction, may be on the order of ten (10), one hundred (100), one thousand (1,000), ten thousand (10,000), or more.

Due to the large number of elements associated with subgenomic intervals that contribute to the certainty metric determination in the correlation, the elements may be “binned” or aggregated by subgenomic interval position or other characteristics in some examples. Binning may avoid a single (or small set of) element(s) disproportionately weighting a correlation in the certainty metric, adversely affecting the estimated tumor fraction. For example, if one element at a single subgenomic interval represents a copy variant with 5,000 copies, it may result in an estimated tumor fraction that is inaccurately high. Therefore, in some examples, elements that contribute to a certainty metric are averaged or otherwise aggregated by chromosome, for example, for each of 22 relevant chromosomes. Those 22 aggregate chromosome values can then be used to calculate the certainty metric which is then correlated with tumor fraction, ensuring that a single subgenomic interval (e.g., SNP site) does not disproportionately affect the correlation. Other methods can be utilized to limit the effect of extreme copy-number events, such as, but not limited to, excluding outlier values from the certainty metric determinations.

In some examples, the correlation may be a mean (i.e., average) correlation, with upper bound correlations and lower bound correlations also calculated. In this way, the mean correlation is bounded by a 95% confidence interval.

The subgenomic interval may comprise one or several subgenomic intervals, and in some examples may be at least one heterozygous SNP site. Subgenomic intervals may be selected based on various criteria. For example, subgenomic intervals may be selected based on how polymorphic the subgenomic interval is in a general healthy population, as well as, bealthy subpopulations (including different genders, ages or ethnic backgrounds). It may be advantageous that the subgenomic intervals vary considerably in the healthy population. The sequencing characteristics of the subgenomic intervals may also be selected on the basis of being “well-behaved,” i.e., near expected allele-frequencies, such as 0, 0.5, and 1.0. Furthermore, the regions may be selected on the basis of being “well covered,” i.e., having typical coverage across populations for the site. Subgenomic intervals may be excluded if they occur in simple repeats of gene families or in any generally repeating sequence of DNA, since this characteristic can challenge alignment methodologies. In an embodiment, subgenomic intervals may be located in a genomic region that is free, or essentially free, of high homology, simple repeats, or gene families.

In an embodiment, the subgenomic interval comprises a minor allele. As used herein, a “minor allele” is an allele other than the most common allele (e.g., the second most common allele or the least common allele) associated with a particular subgenomic interval in a given population. In an embodiment, at least 10, 20, 50, 100, 150, 200, 300, 400, 500, 600, 700, 800, 900, 1000, 1200, 1400, 1600, 1800, 2000, or 10000 heterozygous subgenomic intervals are selected. In one example, no more than 10, 20, 50, 100, 150, 200, 300, 400, 500, 600, 700, 800, 900, 1000, 1200, 1400, 1600, 1800, 2000, or 10000 heterozygous SNP sites are selected.

In one example, the selected subgenomic intervals and/or correlation may be universal, i.e., across all disease ontologies, in order to provide a broad screening technique. In other examples, subgenomic intervals may be selected, and the correlation tuned, based on disease ontology (e.g., tumor type).

One or more certainty metrics may be used in correlating a target variable (e.g., allele coverage deviation and/or allele fraction variation) to tumor fraction. For example, metrics relating to allele fraction may be applied. In one example, an allele frequency entropy metric or root mean squared deviation (RMSD) metric may be used:

$$S [ P ( af ) ] = {\sum }_{af = 0}^{0.5} P ( af ) {log}_{2} ( P ( af ) ) \qquad Allele Frequency Entropy$$

$$RMSD = {[ \frac{1}{N} {\sum }_{i = 0}^{N} {( {af}^{i} - 0.5 )}^{2} ]}^{( 1 / 2 )} \qquad Root Mean Squared Deviation$$

where i=SNP bin and af=allele frequency on the range of 0 to 0.5. Folded SNP allele frequencies are used here by convention (e.g., as described in Nielsen. Hum Genomics. 2004; 1 (3): 218-224 and Marth, et al. Genetics. 2004; 6 (1): 351-372), but the methodology holds if the full range of 0 to 1 is utilized. Other metrics may also be used, such as metrics based off the log 2 ratio. Any of these metrics may incorporate factors such as coverage at a particular SNP bin, where the “bin” can be defined to be 1 or more base-pairs. In some embodiments, the certainty metric may be written as a function of coverage, such that certainty_metric=f(Cvg). Further, any mathematical transformation or operation acting on the certainty_metric, may also be considered a certainty_metric.

In some examples, the certainty metric may be a deviation from the expected log₂ ratio for at least one subgenomic interval. In other examples, the certainty metric may be a deviation from expected allele fraction in a healthy population for at least one subgenomic interval (e.g., a SNP) that is known to be heterozygous. In other examples, the certainty metric may be a deviation from expected allele coverage in the healthy population for at least one subgenomic interval (e.g., a SNP) that is known to be heterozygous.

Table II shows exemplary certainty metrics that may be used, including any p-moment or combination thereof:

TABLE II

| Conditions to Relate | | |
|---|---|---|
| Metric to Tumor | | |
| Fraction | Metric Calculating Relative Certainty of Variable | Comments |
| INTRA-SAMPLE- | | |
| Comparisons | | |
| • All coverage under | variable = af | • Intra-sample comparison |
| consideration is from the cancer sample | $af = \frac{{Cvg}_{a}}{{Cvg}_{a} + {Cvg}_{b}}$ | • Cvgₐ < Cvg_{b}, so that af ≤ 0.5 |
| • af is a metric which | af_{expectation} = 0.5 | |
| compares coverages | | |
| from the maternal and | | |
| paternal chromosomes | | |
| • Cvgₐ is maternal or | | |
| paternal, Cvg_{b} is the | | |
| other allele. | | |
| • N is a number of subgenomic intervals, and i is an index of N. | $certainty\_metric = \sqrt{\frac{1}{N} {\sum }_{i}^{N} {( af - {af}_{expectation} )}^{2}}$ | |
| • Any loci may be used such that the germline maternal and paternal | $= \sqrt{\frac{1}{N} {\sum }_{i}^{N} {( \frac{{Cvg}_{i , a}}{{Cvg}_{i , a} + {Cvg}_{i , b}} - 0.5 )}^{2}}$ | |
| chromosomes differ at | | |
| the same genomic | | |
| location; could be a SNP | | |
| or something larger. | | |
| • All coverage under | variable = Cvg | • Intra-sample comparison |
| consideration is from the cancer sample | | • Cogₐ > Cvg_{b} |
| • Cvgₐ is maternal or paternal, Cvg_{b} is the | $relative\_difference = \frac{{Cvg}_{a} - {Cvg}_{b}}{{Cvg}_{b}}$ | • Previous choice is chosen because we assume the allele is |
| other allele. | relative_difference_{expectation} = 0 | amplified and thus the “b” allele |
| • Any loci may be used such that the germline maternal and paternal | $certainty\_metric = \sqrt{\frac{1}{N} {\sum }_{i}^{N} {( \frac{{Cvg}_{ia} - {Cvg}_{ib}}{{Cvg}_{ib}} )}^{2}}$ | would be the normal. In general, it should not matter since this will switch for deletions. Thus, Cvgₐ |
| chromosomes differ at | | could be < Cvg_{b}, or one could |
| the same genomic | | completely ignore the convention |
| location; could be a SNP | | |
| or something larger. | | |
| • All coverage under | variable = af | • Intra-sample comparison |
| consideration is from the | | • S = Entropy is a metric that |
| cancer sample | | inherently measures relative |
| • af is a metric which | probability of allele frequency = P(af) | certainty of the variable |
| compares coverages from the maternal and | $= P ( \frac{{Cvg}_{i , a}}{{Cvg}_{i , a} + {Cvg}_{i , b}} )$ | |
| paternal chromosomes | | |
| • Cvgₐ is maternal or | certainty_metric = S[P(af)] | |
| paternal, Cvg_{b} is the | | |
| other allele. | | |
| •· Any loci may be used | | |
| such that the germline | | |
| maternal and paternal | | |
| chromosomes differ at | | |
| the same genomic | | |
| location; could be a SNP | | |
| or something larger. | | |
| INTER- SAMPLE- | | |
| Comparisons | | |
| • All coverage under | variable = log2ratio = 12r | • Inter-sample comparison |
| consideration is from both the cancer and reference sample | $ratio = \frac{( C v {g}_{a}^{cancer} + {Cvg}_{b}^{cancer} )}{( Cv {g}_{a}^{normal} + {Cvg}_{b}^{normal} )}$ | |
| • ratio is determined | 12r = log2(ratio) | |
| from the total coverage | 12r_{expectation} = 0 | |
| from maternal and | | |
| paternal from cancer | | |
| sample to the maternal | | |
| and paternal from the | | |
| reference. | | |
| • Any loci of the genome from 1 to an infinite set of bases can be used. | $certainty\_metric = \sqrt{\frac{1}{N} {\sum }_{i}^{N} {[ l 2 r - l 2 {r}_{expectation} ]}^{2}}$ | |
| | $= \sqrt{\frac{1}{N} {\sum }_{i}^{N} {[ ( {log}_{2} \frac{( C v {g}_{i , a}^{cancer} + {Cvg}_{i , b}^{cancer} )}{( Cv {g}_{i , a}^{normal} + {Cvg}_{i , b}^{normal} )} ) - 0 ]}^{2}}$ | |
| • All coverage under | variable = Cvg | • Inter-sample comparison |
| consideration is from both the cancer and | ${Cvg}_{cancer} = ( {Cvg}_{a}^{cancer} + {Cvg}_{b}^{cancer} )$ | |
| reference sample | | |
| • ratio is determined from the total coverage | ${Cvg}_{normal} = ( {Cvg}_{a}^{normal} + {Cvg}_{b}^{normal} )$ | |
| from maternal and paternal from cancer | $relative\_difference = \frac{{Cvg}_{cancer} - {Cvg}_{normal}}{{Cvg}_{normal}}$ | |
| sample to the maternal | | |
| and paternal from the | relative_difference_{expectation} = 0 | |
| reference. | | |
| • Any loci of the genome from 1 to an infinite set of bases can be used. | $certainty\_metric = \sqrt{\frac{1}{N} {\sum }_{i}^{N} {( \frac{{Cvg}_{i , cancer} - {Cvg}_{i , normal}}{{Cvg}_{i , normal}} )}^{2}}$ | |
| • Total coverage from | variable = log2ratio = 12r | • Inter-sample comparison |
| maternal and paternal | probability of log2ratio = P((2r) | • S = Entropy is a metric that does |
| from cancer sample is compared to the maternal and paternal from the | $= P ( {log}_{2} ( \frac{{Cvg}_{i , a}^{cancer} + {Cvg}_{i , b}^{cancer}}{{Cvg}_{i , a}^{normal} + {Cvg}_{i , b}^{normal}} ) )$ | not calculate a deviation from expectation. It is a metric that inherently measures relative |
| reference. | | certainty of the variable |
| • Any loci of the genome | certainty_metric = S[P(12r)] | |
| from 1 to an infinite set | | |
| of bases can be used | | |

At step 810, the tumor fraction of the sample is determined (e.g., estimated) with reference to the certainty metric and the determined relationship. In some examples, the coefficients of the determined relationship are applied to the certainty metric determined from the patient sample, and the products summed to arrive at an evaluated (e.g., estimated) tumor fraction. It will be appreciated that other functions may be performed to yield a final estimated tumor fraction. For example, the estimated tumor fraction may be scaled, normalized, or otherwise adjusted from an initial or raw estimated tumor fraction measure.

At step 812, process 800 ends.

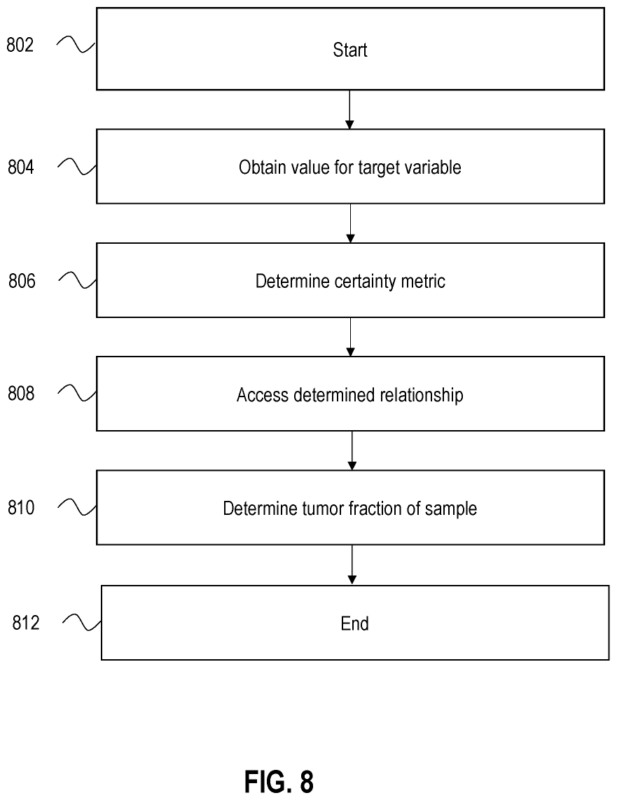

FIG. 9 provides another non-limiting example of a process 900 for determining the tumor fraction in a sample according to embodiments of the present disclosure. Sequencing data (e.g., cell-free DNA (efDNA) sequencing data obtained using a CGP assay) representing values for, e.g., allele fraction, at a plurality of loci within a genome or subgenomic interval of a subject may be processed according to a first tumor fraction estimation process 902 (e.g., the tumor fraction estimator (TFE) process 800 described in FIG. 8). In some instances, the values derived from the sequencing data may represent, e.g., a difference between an allele coverage of a locus in a tumor sample and an allele coverage of the same locus in a non-tumor sample at the plurality of loci within the genome or subgenomic interval of the subject. The estimate of tumor fraction (e.g., a circulating tumor fraction) for the sample returned by the first stage determination is compared to a first threshold, 904. The first threshold may be, for example, a limit-of-detection (LoD) or specified confidence level for determining tumor fraction using the first stage determination. If the estimated tumor fraction returned by the first stage determination is greater than the first threshold, the estimate is output as the determined value of the tumor fraction for the sample, 906. If the estimated tumor fraction returned by the first stage determination is less than or equal to the first threshold, a secondary process may be used to calculate tumor fraction for the sample, 908. In some instances, the secondary method 908 may comprise the use of, for example, a maximum somatic allele frequency (MSAF) determination to estimate tumor fraction of the sample. In some instances, the use of two complementary processes in a composite methodology for determining tumor fraction provides for more accurate determinations of tumor fraction over a larger range of DNA concentrations (e.g., circulating tumor DNA (ctDNA) concentrations).

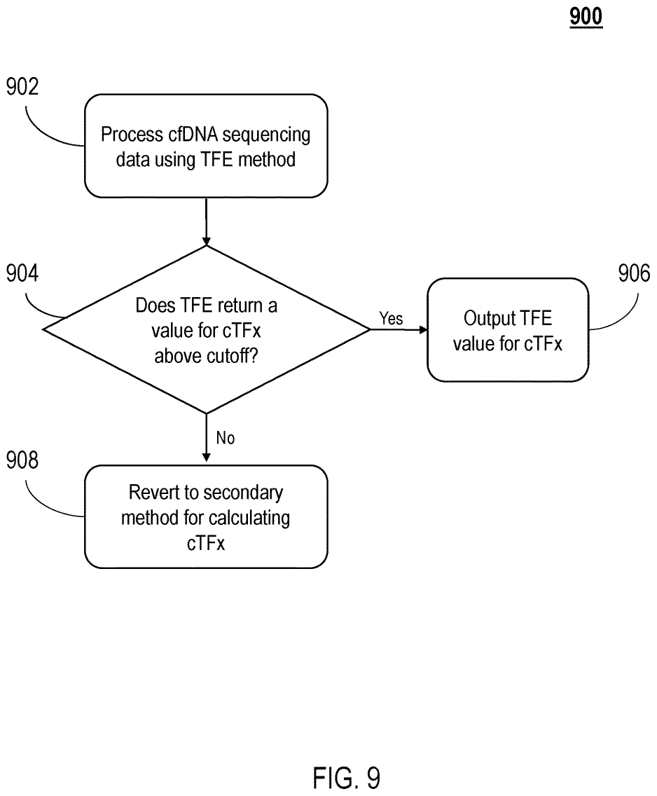

FIG. 10 provides another non-limiting example of a process 1000 for determining tumor fraction according to embodiments of the present disclosure. Sequencing data (e.g., cell-free DNA (cfDNA) sequencing data) representing values for, e.g., allele fraction or a difference between an allele coverage of a locus in a tumor sample and an allele coverage of the same locus in a non-tumor sample, at a plurality of loci within a genome or subgenomic interval of a subject may be processed according to a first tumor fraction estimation process 1002 (e.g., the tumor fraction estimator (TFE) process 800 described in FIG. 8). The estimate of tumor fraction (e.g., a circulating tumor fraction) for the sample returned by the first stage determination is compared to a first threshold, 1004. The first threshold may be, for example, a limit-of-detection (LoD) or specified confidence level for determining tumor fraction using the first stage determination. If the estimated tumor fraction returned by the first stage determination is greater than the first threshold, the estimate is output as the determined value of the tumor fraction for the sample, 1006. If the estimated tumor fraction returned by the first stage determination is less than or equal to the first threshold, the sequencing data may be examined for quality control issues, 1008. For example, a quality metric may be calculated for the sequencing data and compared to a quality control threshold (e.g., a second threshold). Examples of quality control parameters that may be examined, used as a quality metric, and/or used to calculate a quality metric for the sequencing data include, but are not limited to, an average sequence coverage for the sample, a minimum average sequence coverage for the sample, an allele coverage at each of the corresponding loci in the plurality of loci, a minimum allele coverage at each of the corresponding loci in the plurality of loci, a degree of nucleic acid contamination in the sample (determined, e.g., by quantifying aberrations in SNP allele frequencies), a maximum degree of nucleic acid contamination in the sample, a number of single nucleotide polymorphism (SNP) loci within the plurality of loci examined a minimum number of single nucleotide polymorphism (SNP) loci within the plurality of loci examined, or any combination thereof. In some instances, the quality control threshold (or second threshold) may comprise a specified lower limit of the quality metric.

In the case that no quality control issues are identified for the sequencing data (e.g., the quality metric for the sequencing data is greater than a specified quality threshold), a first version of a secondary method 1010 may be used to calculate tumor fraction for the sample. In some instances, the secondary method 1010 may comprise, for example, a first determination of an allele frequency (e.g., a maximum somatic allele frequency (MSAF1)) to estimate tumor fraction of the sample. In the case that quality control issues are identified for the sequencing data (e.g., the quality metric for the sequencing data is less than or equal to a specified quality threshold), a second version of the secondary method 1012 may be used to calculate tumor fraction for the sample. In some instances, the secondary method 1012 may comprise, for examples, a second determination of an allele frequency (e.g., a maximum somatic allele frequency (MSAF2)) to estimate tumor fraction for the sample.

In some instances, the disclosed methods may be used to identify variants in the ABLI, ACVRIB, AKTI, AKT2, AKT3, ALK, ALOX12B, AMERI, APC, AR, ARAF, ARFRP1, ARIDIA, ASXL1, ATM, ATR, ATRX, AURKA, AURKB, AXIN1, AXL, BAP1, BARDI, BCL2, BCL2L1, BCL2L2, BCL6, BCOR, BCORLI, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKNIA, CDKNIB, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNFIA, HRAS, HSD3B1, ID3, IDHI, IDH2, IGFIR, IKBKE, IKZF1, INPP4B, IRF2, IRF4, IRS2, JAK1, JAK2, JAK3, JUN, KDMSA, KDM5C, KDM6A, KDR, KEAPI, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKB1A, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCDILG2, PDGFRA, PDGFRβ, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKARIA, PRKCI, PTCH1, PTEN, PTPN11, PTPRO, QKI, RACI, RAD21, RAD51, RAD51B, RADSIC, RAD51D, RAD52, RAD54L, RAFI, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCIL1, WT1, XPO1, XRCC2, ZNF217, or ZNF703 gene locus, or any combination thereof.

In some instances, the disclosed methods may be used to identify variants in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CSI, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3K8, PIGF, PTCH, RAF, RANKL, RET, ROS1, SLAMF7, VEGF, VEGFA, or VEGFB gene locus, or any combination thereof.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for determining a loss of heterozygosity (LOH) status of one or more HLA-I genes may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for determining a LOH status of one or more HLA-I genes may be used to predict genetic disorders in fetal DNA. (e.g., for invasive or non-invasive prenatal testing). For example, sequence read data obtained by sequencing fetal DNA extracted from samples obtained using invasive amniocentesis, chorionic villus sampling (cVS), or fetal umbilical cord sampling techniques, or obtained using non-invasive sampling of cell-free DNA (cfDNA) samples (which comprises a mix of maternal cfDNA and fetal cfDNA), may be processed according to the disclosed methods to identify variants, e.g., copy number alterations, associated with, e.g., Down Syndrome (trisomy 21), trisomy 18, trisomy 13, and extra or missing copies of the X and Y chromosomes.

In some instances, the disclosed methods for determining a LOH status of one or more HLA-I genes may be used to select a subject (e.g., a patient) for a clinical trial based on the HLA-I LOH status determined for one or more gene loci. In some instances, patient selection for clinical trials based on, e.g., identification of HLA-I LOH status at one or more gene loci, may accelerate the development of targeted therapies and improve the healthcare outcomes for treatment decisions.

In some instances, the disclosed methods for determining a LOH status of one or more HLA-I genes may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapeutic agent, radiation therapy, a targeted therapy (e.g., immunotherapy), surgery, or any combination thereof.

In some instances, the targeted therapy (or anti-cancer target therapy) may comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Pigray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the disclosed methods for determining a LOH status of one or more HLA-I genes may be used in treating a disease (e.g., a cancer) in a subject. For example, in response to determining an HLA-I LOH status using any of the methods disclosed herein, an effective amount of an anti-cancer therapy or anti-cancer treatment may be administered to the subject.

In some instances, the disclosed methods for determining a LOH status of one or more HLA-I genes may be used for monitoring disease progression or recurrence (e.g., cancer or tumor progression or recurrence) in a subject. For example, in some instances, the methods may be used to determine an HLA-I LOH status in a first sample obtained from the subject at a first time point, and used to determine an HLA-I LOH status in a second sample obtained from the subject at a second time point, where comparison of the first determination of an HLA-I LOH status and the second determination of an HLA-I LOH status allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determination of an HLA-I LOH status.

In some instances, the HLA-I LOH status determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for determining a LOH status of one or more HLA-I genes may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for determining a LOH status of one or more HLA-I genes as part of a genomic profiling process (or inclusion of the output from the disclosed methods for determining a LOH status of one or more HLA-I genes as part of the genomic profile of the subject) can improve the validity of, e.g., disease detection calls and treatment decisions, made on the basis of the genomic profile by, for example, independently determining a LOH status of one or more HLA-I genes in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

In some instances, embodiments of the present disclosure comprise methods for treating a subject having a cancer, comprising: determining a HLA-I LOH status and treating the subject with a non-immune-oncology (IO) therapy if the subject is determined to have an HLA-I LOH positive status; or treating the subject with an IO therapy if the subject is determined to have an HLA-I LOH non-positive status.

In some instances, embodiments of the present disclosure comprise methods for selecting a treatment for a subject having a cancer, the method comprising: determining a HLA-I LOH status, identifying the subject for treatment with an IO therapy if the subject is determined to have a HLA-I LOH non-positive status and identifying the subject for treatment with a non-IO therapy if the subject is determined to have the HLA-I LOH positive status.

In some instances, embodiments of the present disclosure comprise methods for identifying a subject having a cancer for treatment with an immune oncology (IO) therapy, the method comprising: determining a HLA-I LOH status, identifying the subject for treatment with an IO therapy if the subject is determined to have a HLA-I LOH non-positive status, and identifying the subject for treatment with a non-IO therapy if the subject is determined to have the HLA-I LOH positive status.

In some instances, embodiments of the present disclosure comprise methods of identifying one or more treatment options for a subject having a cancer, the method comprising: determining a HLA-I LOH status, generating a report comprising one or more treatment options identified for the subject based at least in part on the HLA-I LOH status determined for the sample, wherein: the subject is identified as one who may benefit from treatment with a non-IO therapy if the subject is determined to have a HLA-I LOH positive status and the subject is identified as one who may benefit from treatment with an IO therapy if the subject is determined to have a HLA-I LOH non-positive status.

In some instances, embodiments of the present disclosure comprise methods of stratifying a subject having cancer for treatment with an immuno-oncology (IO) therapy, the method comprising determining an HLA-I LOH status, treating the subject with a non-IO therapy if the subject is determined to have an HLA-I LOH positive status, treating the subject with an IO therapy if the subject is determined to have an HLA-I LOH non-positive status and treating the subject with a non-IO therapy if the subject is determined to have an HLA-I LOH positive status.

In some instances, embodiments of the present disclosure comprise methods of predicting survival of a subject having cancer, the method comprising: determining an HLA-I LOH status, wherein: if the subject is determined to have a HLA-I LOH positive status, the subject is predicted to have shorter survival when treated with an IO therapy, as compared to a subject that was determined to have an HLA-I LOH non-positive status, and if the subject is determined to have an HLA-I LOH non-positive status, the subject is predicted to have longer survival when treated with an IO therapy, as compared to a subject that was determined to have an HLA-I LOH positive status.

Embodiments of the present disclosure comprise methods of predicting survival of a subject having cancer, the method comprising: determining a HLA-I LOH status, wherein: if the subject is determined to have the HLA-I LOH positive status, the subject is predicted to have longer survival when treated with a non-immuno-oncology (IO) therapy, as compared to a subject that was treated with an IO therapy.

In one or more embodiments, the methods described above further comprise determining a tumor mutational burden (TMB) in the sample from the subject, wherein predicted survival is further based on the the TMB. In one or more embodiments of the methods described above, the subject is determined to have a TMB of at least about 4 to 100 mutations/Mb, about 4 to 30 mutations/Mb, 8 to 100 mutations/Mb, 8 to 30 mutations/Mb, 10 to 20 mutations/Mb, less than 4 mutations/Mb, or less than 8 mutations/Mb. In one or more embodiments of the methods described above, the TMB is at least about 5 mutations/Mb, is at least about 10 mutations/Mb, at least about 12 mutations/Mb, at least about 16 mutations/Mb, at least about 20 mutations/Mb, or at least about 30 mutations/Mb. In one or more embodiments of the methods described above, the TMB is determined based on between about 100 kb to about 10 Mb. In one or more embodiments of the methods described above, the TMB is determined based on between about 0.8 Mb to about 1.1 Mb.

In one or more embodiments of the methods described above, the IO therapy comprises a single IO agent or multiple IO agents. In one or more embodiments of the methods described above, the IO therapy comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a cellular therapy, a treatment for cancer being tested in a clinical trial, an immunotherapy, or any combination thereof.

In one or more embodiments of the methods described above, the IO therapy comprises an immune checkpoint inhibitor. In one or more embodiments, the immune checkpoint inhibitor is a PD-1 inhibitor. In one or more embodiments, the immune checkpoint inhibitor comprises one or more of nivolumab, pembrolizumab, cemiplimab, or dostarlimab. In one or more embodiments, the immune checkpoint inhibitor is a PD-L1-inhibitor. In one or more embodiments, the immune checkpoint inhibitor comprises one or more of atezolizumab, avelumab, or durvalumab. In one or more embodiments, the immune checkpoint inhibitor is a CTLA-4 inhibitor. In one or more embodiments, the CTLA-4 inhibitor comprises ipilimumab. In one or more embodiments, the nucleic acid comprises a double-stranded RNA (dsRNA), a small interfering RNA (siRNA), or a small hairpin RNA (shRNA). In one or more embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In one or more embodiments, the methods described above further comprise treating the subject determined to have an HLA-I LOH non-positive status with the IO therapy. In one or more embodiments of the methods described above, the non-IO therapy comprises one or more of a small molecule inhibitor, a chemotherapeutic agent, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

In one or more embodiments, the chemotherapeutic agent comprises one or more of an alkylating agent, an alkyl sulfonates aziridine, an ethylenimine, a methylamelamine, an acetogenin, a camptothecin, a bryostatin, a callystatin, CC-1065, a cryptophycin, aa dolastatin, a duocarmycin, a eleutherobin, a pancratistatin, a sarcodictyin, a spongistatin, a nitrogen mustard, a nitrosureas, an antibiotic, a dynemicin, a bisphosphonate, an esperamicina a neocarzinostatin chromophore or a related chromoprotein enediyne antiobiotic chromophore, an anti-metabolite, a folic acid analogue, a purine analog, a pyrimidine analog, an androgens, an anti-adrenal, a folic acid replenisher, aldophosphamide glycoside, aminolevulinic acid, eniluracil, amsacrine, bestrabucil, bisantrene, edatraxate, defofamine, demecolcine, diaziquone, elformithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, a PSK polysaccharide complex, razoxane, rhizoxin, sizofiran, spirogermanium, tenuazonic acid, triaziquone, 2,2′,2″-trichlorotriethylamine, a trichothecene, urethan, vindesine, dacarbazine, mannomustine, mitobronitol, mitolactol, pipobroman, gacytosine, arabinoside (“Ara-C”), cyclophosphamide, a taxoid, 6-thioguanine, mercaptopurine, a platinum coordination complex, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluorometlbylomithine (DMFO), a retinoid, capecitabine, carboplatin, procarbazine, plicomycin, gemcitabine, navelbine, farnesyl-protein transferase inhibitors, transplatinum, or any combination thereof.

In one or more embodiments, the methods described above further comprise treating the subject determined to have an HLA-I LOH positive status with the non-IO therapy. In one or more embodiments, the methods described above further comprise treating the subject with an additional anti-cancer therapy. In one or more embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

In one or more embodiments of the methods described above, the survival is a progression-free survival, an overall survival, a disease-free survival (DFS), an objective response rate (ORR), a time to tumor progression (TTP), a time to treatment failure (TTF), a durable complete response (DCR), or a time to next treatment (TTNT).

In one or more embodiments of the methods described above, the sample comprises a tissue biopsy sample or a liquid biopsy sample. In such embodiments, the sample is a tissue biopsy and comprises a tumor biopsy, tumor specimen, or circulating tumor cells. In one or more embodiments, the sample is a liquid biopsy sample and comprises blood, serum, plasma, cerebrospinal fluid, sputum, stool, urine, or saliva.

In one or more embodiments of the methods described above, the sample comprises cells and/or nucleic acids from the cancer. In such embodiments, the sample comprises mRNA, DNA, circulating tumor DNA (ctDNA), cell-free DNA, cell-free RNA from the cancer, or any combination thereof. In one or more embodiments, the sample is a liquid biopsy sample and comprises circulating tumor cells (CTCs). In one or more embodiments, the sample is a liquid biopsy sample and comprises cell-free DNA (cfDNA), circulating tumor DNA (ctDNA), or any combination thereof.

In one or more embodiments of the methods described above, the LOH status of the gene is determined based on sequencing read data derived from sequencing the sample from the subject. In such embodiments, the sequencing comprises use of a massively parallel sequencing (MPS) technique, whole genome sequencing (WGS), whole exome sequencing, targeted sequencing, direct sequencing, next-generation sequencing (NGS), or a Sanger sequencing technique. In one or more embodiments, the sequencing comprises: providing a plurality of nucleic acid molecules obtained from the sample, wherein the plurality of nucleic acid molecules comprises a mixture of tumor nucleic acid molecules and non-tumor nucleic acid molecules; optionally, ligating one or more adapters onto one or more nucleic acid molecules from the plurality of nucleic acid molecules; amplifying nucleic acid molecules from the plurality of nucleic acid molecules; optionally, capturing nucleic acid molecules from the amplified nucleic acid molecules, wherein the captured nucleic acid molecules are captured from the amplified nucleic acid molecules by hybridization to one or more bait molecules; and sequencing, by a sequencer, the captured nucleic acid molecules to obtain a plurality of sequence reads corresponding to one or more genomic loci within a subgenomic interval in the sample.

In one or more embodiments, the adapters comprise one or more of amplification primer sequences, flow cell adapter hybridization sequences, unique molecular identifier sequences, substrate adapter sequences, or sample index sequences. In one or more embodiments, amplifying nucleic acid molecules comprises performing a polymerase chain reaction (PCR) technique, a non-PCR amplification technique, or an isothermal amplification technique. In one or more embodiments, the one or more bait molecules comprise one or more nucleic acid molecules, each comprising a region that is complementary to a region of a captured nucleic acid molecule. In such embodiments, the one or more bait molecules each comprise a capture moiety. In such embodiments, the capture moiety is biotin.

In one or more embodiments of the methods described above, the cancer is a B cell cancer, a melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypereosinophilia, chronic cosinophilie leukemia, neuroendocrine cancers, or a carcinoid tumor.

In some instances, the subject may have been previously treated with an anti-cancer therapy. In such embodiments, the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly (A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) sitail of mature mRNAs by priming with oligo (dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject bas a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypercosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologie malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a bematologie malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell bistiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164 (1): 35-42; Masuda, et al., (1999) Nucleic Acids Res. 27 (22): 4436-4443; Specht, et al., (2001) Am J Pathol. 158 (2): 419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4 (11): 903-5; Hodges, E. et al. (2007) Nat. Genet. 39 (12): 1522-7; and Okou, D. T. et al. (2007) Nat. Methods 4 (11): 907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLID, ION Torrent, Complete Genomies, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform “, Bioinformatics epub. PMID: 20080505), the Smith-Waterman algorithm (see, e.g., Smith, et al. (1981), “Identification of Common Molecular Subsequences”, J. Molecular Biology 147 (1): 195-197), the Striped Smith-Waterman algorithm (see, e.g., Farrar (2007), “Striped Smith-Waterman Speeds Database Searches Six Times Over Other SIMD Implementations”, Bioinformatics 23 (2): 156-161), the Needleman-Wunsch algorithm (Needleman, et al. (1970) “A General Method Applicable to the Search for Similarities in the Amino Acid Sequence of Two Proteins”, J. Molecular Biology 48 (3): 443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (1) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26 (5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85 (6): 847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ~le-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20 (9): 1297-303; Ye, K., et al., Bioinformatics, 2009; 25 (21): 2865-71; Lunter, G., and Goodson, M., Genome Res. 2011; 21 (6): 936-9; and Li, H., et al. (2009), Bioinformatics 25 (16): 2078-9.

Methods for generating indel calls and individual-level genotype likelihoods include, e.g., the Dindel algorithm (Albers, C. A., et al., Genome Res. 2011; 21 (6): 961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21 (6): 952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26 (6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of mutation calling methods is provided in, e.g., International Patent Application Publication No. WO 2020/236941, the entire content of which is incorporated herein by reference.

### Systems

Also disclosed herein are systems designed to implement any of the disclosed methods for determining a LOH status of one or more HLA-I genes in a sample from a subject. The systems may comprise, e.g., one or more processors, and a memory unit communicatively coupled to the one or more processors and configured to store instructions that, when executed by the one or more processors, cause the system to: identify a plurality of genomic segments in a sample; select from the plurality of genomic segments, one or more genomic segments that overlap with one or more HLA-I genes in the sample; determine whether a tumor purity of the sample is in a predetermined range; when the tumor purity of the sample is in the predetermined range, obtain a copy number value of the one or more selected genomic segments, and determine a LOH status of the one or more HLA-I genes based on the copy number value.

In some embodiments, the systems may comprise, e.g., one or more processors, and a memory unit communicatively coupled to the one or more processors and configured to store instructions that, when executed by the one or more processors, cause the system to: receive, at the one or more processors, one or more reads corresponding to a plurality of genomic segments in a sample, wherein the one or more reads are aligned with a reference sequence. The system can further cause the one or more processors to determine: whether a number of one or more reads exceeds a predetermined read threshold; whether the plurality of genomic segments are germline heterozygous at one or more HLA-I genes in the sample; and whether a tumor content of the sample is above a predetermined tumor content threshold. The system can further cause the one or more processors to determine a gene minor allele frequency (MAF) of the sample and a segment MAF of the sample when the plurality of genomic segments are germline heterozygous, the number of reads in the one or more reads exceeds the predetermined read threshold, and the tumor content is above the predetermined tumor content threshold. The system can further cause the one or more processors to determine a loss of heterozygosity (LOH) status of the sample based on the gene MAF of the sample, the segment MAF of the sample, and the tumor content of the sample.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLID) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for determining an HLA-I LOH status in any of a variety of samples as described herein (e.g., a tissue sample, biopsy sample, hematological sample, or liquid biopsy sample derived from the subject).

In some instances, the plurality of gene loci for which sequencing data is processed to determine an HLA-I LOH status may comprise at least 1, 2, 3, 4, 5, 6, 7, 8, 9, 10, or more than 10 gene loci.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of an HLA-I LOH status is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

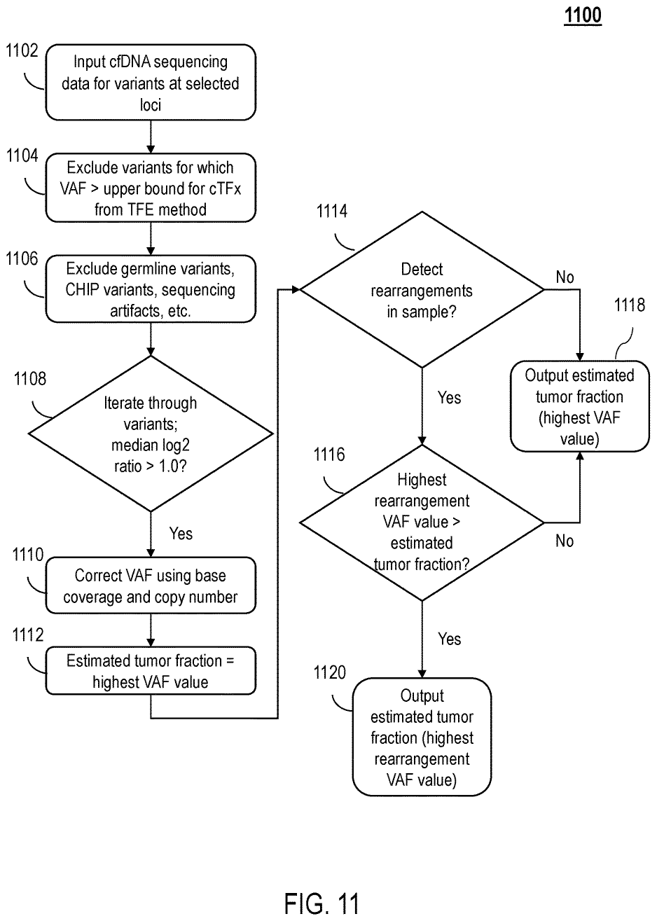

FIG. 12 illustrates an example of a computing device or system in accordance with one embodiment. Device 1200 can be a host computer connected to a network. Device 1200 can be a client computer or a server. As shown in FIG. 12, device 1200 can be any suitable type of microprocessor-based device, such as a personal computer, workstation, server or handheld computing device (portable electronic device) such as a phone or tablet. The device can include, for example, one or more processor(s) 1210, input devices 1220, output devices 1230, memory or storage devices 1240, communication devices 1260, and nucleic acid sequencers 1270. Software 1250 residing in memory or storage device 1240 may comprise, e.g., an operating system as well as software for executing the methods described herein. Input device 1220 and output device 1230 can generally correspond to those described herein, and can either be connectable or integrated with the computer.

Input device 1220 can be any suitable device that provides input, such as a touch screen, keyboard or keypad, mouse, or voice-recognition device. Output device 1230 can be any suitable device that provides output, such as a touch screen, haptics device, or speaker.

Storage 1240 can be any suitable device that provides storage (e.g., an electrical, magnetic or optical memory including a RAM (volatile and non-volatile), cache, hard drive, or removable storage disk). Communication device 1260 can include any suitable device capable of transmitting and receiving signals over a network, such as a network interface chip or device. The components of the computer can be connected in any suitable manner, such as via a wired media (e.g., a physical system bus 1280, Ethernet connection, or any other wire transfer technology) or wirelessly (e.g., Bluetooth®, Wi-Fi®, or any other wireless technology).

Software module 1250, which can be stored as executable instructions in storage 1240 and executed by processor(s) 1210, can include, for example, an operating system and/or the processes that embody the functionality of the methods of the present disclosure (e.g., as embodied in the devices as described herein).

Software module 1250 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 1240, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 1250 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

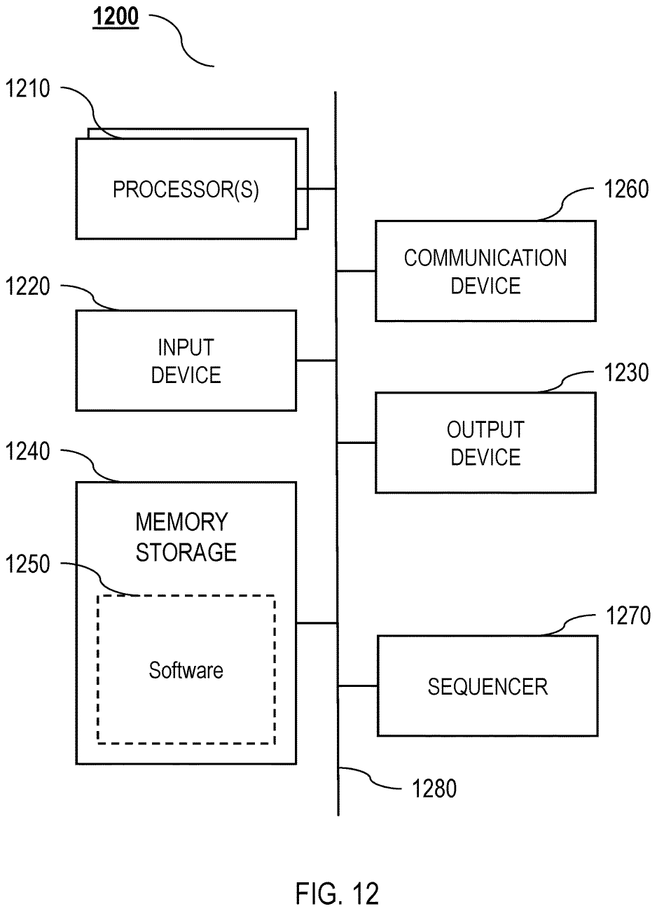

Device 1200 may be connected to a network (e.g., network 1304, as shown in FIG. 13 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1200 can be implemented using any operating system, e.g., an operating system suitable for operating on the network. Software module 1250 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 1210.

Device 1200 can further include a sequencer 1270, which can be any suitable nucleic acid sequencing instrument.

FIG. 13 illustrates an example of a computing system in accordance with one embodiment. In system 1300, device 1200 (e.g., as described above and illustrated in FIG. 12) is connected to network 1304, which is also connected to device 1306. In some embodiments, device 1306 is a sequencer. Exemplary sequencers can include, without limitation, Roche/454's Genome Sequencer (GS) FLX System, Illumina/Solexa's Genome Analyzer (GA), Illumina's HiSeq@ 2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS system.

Devices 1200 and 1306 may communicate, e.g., using suitable communication interfaces via network 1304, such as a Local Area Network (LAN), Virtual Private Network (VPN), or the Internet. In some embodiments, network 1304 can be, for example, the Internet, an intranet, a virtual private network, a cloud network, a wired network, or a wireless network. Devices 1200 and 1306 may communicate, in part or in whole, via wireless or hardwired communications, such as Ethernet, IEEE 802.11b wireless, or the like. Additionally, devices 1200 and 1306 may communicate, e.g., using suitable communication interfaces, via a second network, such as a mobile/cellular network. Communication between devices 1200 and 1306 may further include or communicate with various servers such as a mail server, mobile server, media server, telephone server, and the like. In some embodiments, Devices 1200 and 1306 can communicate directly (instead of, or in addition to, communicating via network 1304), e.g., via wireless or hardwired communications, such as Ethernet, IEEE 802.11b wireless, or the like. In some embodiments, devices 1200 and 1306 communicate via communications 1308, which can be a direct connection or can occur via a network (e.g., network 1304).

One or all of devices 1200 and 1306 generally include logic (e.g., http web server logic) or are programmed to format data, accessed from local or remote databases or other sources of data and content, for providing and/or receiving information via network 1304 according to various examples described herein.

### EXAMPLES

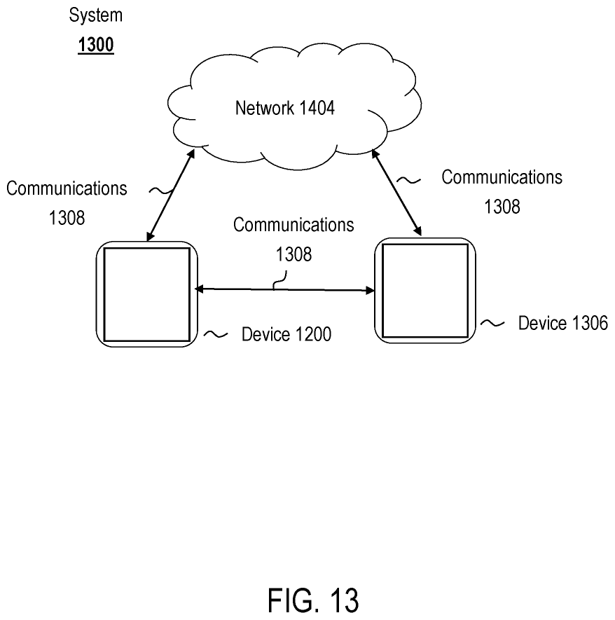

Embodiments of the present disclosure can be used to assess the overall survival of individuals treated with ICIs. FIG. 14 depicts an exemplary plot illustrating the overall survival of individuals with non-squamous cell carcinoma (SCC) non-small cell lung cancer (NSCLC) grouped by a predicted HLA-I LOH status, according to embodiments of the present disclosure. For example, individuals who were treated with an immune checkpoint inhibitor (ICI) were assessed to determine a TMB status and an HLA-I LOH status according to embodiments of this disclosure (e.g., the HLA-I LOH status determined according to process 100A). As shown in the figure, individuals with an HLA-I LOH positive status had a worse overall survival than individuals with an HLA-I LOH non-positive (e.g., negative) status. Additionally, as shown in the figure, the HLA-I LOH positive status reduced the effectiveness of ICI treatment in individuals with a high TMB. Additionally, individuals with low TMB for a particular HLA-I LOH status (e.g., either positive or non-positive) were predicted to have a lower survival than individuals with high TMB for the same HLA-I LOH status. Accordingly, HLA-I LOH status can be used in conjunction with TMB status to predict overall survival of an individual.

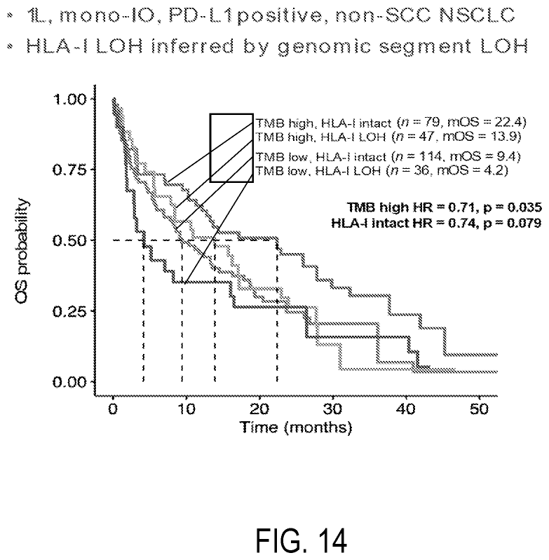

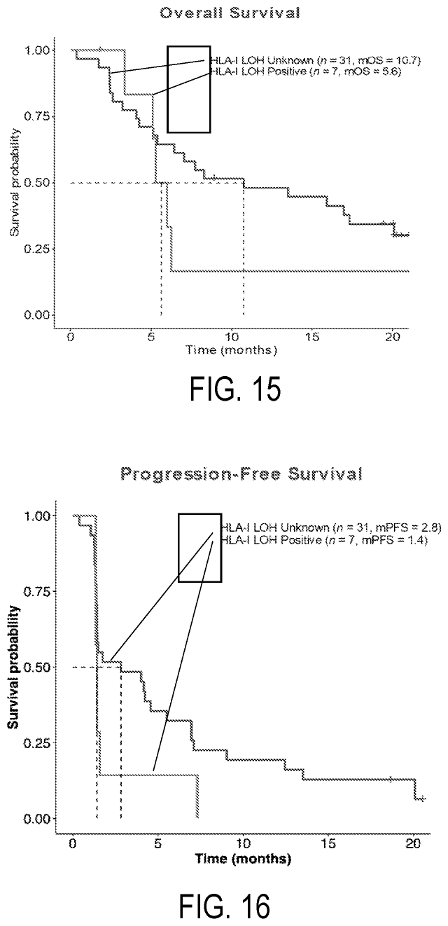

Embodiments of the present disclosure can be used to assess the overall survival and the progression-free survival of individuals treated with ICIs. FIG. 15 depicts an exemplary plot illustrating the overall survival of individuals grouped by a predicted HLA-I LOH status, according to embodiments of the present disclosure (e.g., according to process 100B). FIG. 16 depicts an exemplary plot illustrating the progression-free survival of individuals grouped by a predicted HLA-I LOH status, according to embodiments of the present disclosure (e.g., according to process 100B). The individuals included in the plot were determined to have non-SCC NSCLC, were treated with an ICI (e.g., atezolizumab), and were determined to have high blood TMB (bTMB). As shown in the figures, individuals predicted to have an HLA-I LOH positive status were found to have worse overall survival and worse progression-free survival compared to individuals that had an HLA-I LOH non-positive status.

### EXEMPLARY IMPLEMENTATIONS

Exemplary implementations of the methods and systems described herein include:
1. A method comprising:
   providing a plurality of nucleic acid molecules obtained from a sample from a subject;
   ligating one or more adapters onto one or more nucleic acid molecules from the plurality of nucleic acid molecules;
   amplifying the one or more ligated nucleic acid molecules from the plurality of nucleic acid molecules;
   capturing amplified nucleic acid molecules from the amplified nucleic acid molecules;
   sequencing, by a sequencer, the captured nucleic acid molecules to obtain a plurality of sequence reads that represent the captured nucleic acid molecules;
   receiving, at one or more processors, sequence read data for the plurality of sequence reads;
   identifying, using one or more processors, a plurality of genomic segments in the sample based on the sequence read data;
   selecting, using the one or more processors, from the plurality of genomic segments, one or more genomic segments that overlap with one or more HLA-I genes in the sample;
   determining, using the one or more processors, whether a tumor purity of the sample is in a predetermined range;
   when the tumor purity of the sample is in the predetermined range, obtaining, using the one or more processors, a copy number value of the one or more selected genomic segments; and
   determining, using the one or more processors, a loss of heterozygosity (LOH) status of the one or more HLA-I genes based on the copy number value.
2. The method of clause 1, further comprising obtaining the sample from the subject.
3. The method of clause 1 or clause 2, further comprising:
   performing, using the one or more processors, a quality control procedure prior to obtaining the copy number value; and
   foregoing determining the LOH status of the one or more HLA-I genes in accordance with a determination that the sample is associated with a quality control issue.
4. The method of clause 3, wherein the quality control issue includes a sample contamination, a confirmed transplant, a noisy copy number, or a combination thereof.
5. The method of any of clauses 1 to 4, wherein the predetermined range comprises a tumor purity between 20-95%.
6. The method of any of clauses 1 to 5, wherein the LOH status is determined without baiting the one or more HLA-I genes.
7. The method of any of clauses 1 to 6, wherein the one or more HLA-I genes comprise an HLA-A gene, an HLA-B gene, an HLA-C gene, or a combination thereof.
8. The method any of clauses 1 to 7, further comprising determining a zygosity score based on a copy number of the one or more selected genomic segments.
9. The method of clause 8, wherein a zygosity score of about 1 corresponds to a positive LOH status and a zygosity score of about 2 indicates a non-positive LOH status.
10. The method of any one of clauses 1 to 9, wherein the subject is suspected of having or is determined to have cancer.
11. The method of clause 10, wherein the cancer is a B cell cancer, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
12. The method of clause 10, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.
13. The method of any of clauses 10 to 12, further comprising treating the subject with an anti-cancer therapy based on the LOH status.
14. The method of clause 13, wherein the anti-cancer therapy comprises a targeted anti-cancer therapy.
15. The method of clause 14, wherein the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Pigray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fibj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idbifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), jobenguane I131 (Azedra), ipilimumab (Yervoy), isatuximab-irfe (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.
16. The method of any one of clauses 1 to 15, wherein the sample comprises one or more samples.
17. The method of clauses 1 to 16, wherein the sample comprises a normal tissue sample, a tumor tissue sample, or a liquid biopsy sample.
18. The method of clause 17, wherein the sample is a liquid biopsy sample and comprises blood, plasma, cerebrospinal fluid, sputum, stool, urine, or saliva.
19. The method of clause 17, wherein the sample is a liquid biopsy sample and comprises circulating tumor cells (CTCs), cell-free DNA (cfDNA), circulating tumor DNA (ctDNA), or any combination thereof.
20. The method of any of clauses 1 to 19, wherein determining the copy number value comprises:
   determining, using the one or more processors, a ploidy of the sample, coverage ratio data of the sample, allele fraction data of the sample, segmentation data of the sample, and a copy number model for the one or more HLA-I genes;
   determining, using the one or more processors, segment copy numbers for the one or more selected genomic segments that overlap with the one or more HLA-I genes in the sample based on at least the coverage ratio data, the allele fraction data, the segmentation data, and the copy number model;
   detecting, using the one or more processors, a presence of amplifications or deletions for one or more gene loci of the one or more HLA-I genes based on a segment copy number of the segment copy numbers for a corresponding genomic segment of the one or more selected genomic segments; and
   calling, using the one or more processors, the copy number of the one or more selected genomic segments based on the detected presence of amplifications or deletions for the one or more gene loci of the one or more HLA-I genes.
21. The method of any one of clauses 1 to 20, wherein the plurality of nucleic acid molecules comprises a mixture of tumor nucleic acid molecules and non-tumor nucleic acid molecules.
22. The method of clause 21, wherein the tumor nucleic acid molecules are derived from a tumor portion of a heterogeneous tissue biopsy sample, and the non-tumor nucleic acid molecules are derived from a normal portion of a heterogeneous tissue biopsy sample.
23. The method of clause 21, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
24. The method of any of clauses 1 to 23, wherein the one or more adapters comprise amplification primers, flow cell adaptor sequences, substrate adapter sequences, or sample index sequences.
25. The method of any of clauses 1 to 24, wherein the captured nucleic acid molecules are captured from the amplified nucleic acid molecules by hybridization to one or more bait molecules.
26. The method of clause 25, wherein the one or more bait molecules comprise one or more nucleic acid molecules, each comprising a region that is complementary to a region of a captured nucleic acid molecule.
27. The method of one of clauses 1 to 26, wherein amplifying nucleic acid molecules comprises performing a polymerase chain reaction (PCR) amplification technique, a non-PCR amplification technique, or an isothermal amplification technique.
28. The method of any of clauses 1 to 27, wherein the sequencing comprises use of a massively parallel sequencing (MPS) technique, whole genome sequencing (WGS), whole exome sequencing, targeted sequencing, direct sequencing, or Sanger sequencing technique.
29. The method of clause 28, wherein the sequencing comprises massively parallel sequencing, and the massively parallel sequencing technique comprises next generation sequencing (NGS).
30. The method of any one of clauses 1 to 29, wherein the sequencer comprises a next generation sequencer.
31. The method of any one of clauses 1 to 30, wherein one or more of the sequence reads overlap one or more gene loci within one or more subgenomic intervals in the sample.
32. The method of clause 31, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.
33. The method of clause 31 or clause 32, wherein the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKNIB, CDKN2A, CDKN2B, CDKN2C, CEBPA, CHEK1, CHEK2, CIC, CREBBP, CRKL, CSF1R, CSF3R, CTCF, CINNA1, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNFIA, HRAS, HSD3B1, ID3, IDH1, IDH2, IGFIR, IKBKE, IKZF1, INPP4B, IRF2, IRF4, IRS2, JAK1, JAK2, JAK3, JUN, KDMSA, KDMSC, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKB1A, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCDILG2, PDGFRA, PDGFRβ, PDK1, PIK3C2B, PIK3C2G, PIK3C_{A}, PIK3C_{B}, PIK3R1, PIM1, PMS2, POLD1, POLE, PPARG, PPP2R1A, PPP2R2A, PRDM1, PRKARIA, PRKCI, PTCH1, PTEN, PTPN11, PTPRO, QKI, RAC1, RAD21, RAD51, RADS1B, RAD51C, RADSID, RAD52, RAD54L, RAF1, RARA, R_{B}1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCIL1, WT1, XPO1, XRCC2, ZNF217, ZNF703, or any combination thereof.
34. The method of clause 32 or clause 33, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.
35. The method of any one of clauses 1 to 34, further comprising generating, by the one or more processors, a report indicating the LOH status of the one or more HLA-I genes.
36. The method of clause 35, further comprising transmitting the report to a healthcare provider.
37. The method of clause 36, wherein the report is transmitted via a computer network or a peer-to-peer connection.
38. A method, comprising:
   identifying, using one or more processors, a plurality of genomic segments in a sample;
   selecting, using the one or more processors, from the plurality of genomic segments, one or more genomic segments that overlap with one or more HLA-I genes in the sample;
   determining, using the one or more processors, whether a tumor purity of the sample is in a predetermined range;
   when the tumor purity of the sample is in the predetermined range, obtaining, using the one or more processors, a copy number value of the one or more selected genomic segments; and
   determining, using the one or more processors, a loss of heterozygosity (LOH) status of the one or more HLA-I genes based on the copy number value.
39. The method of clause 38, further comprising:
   performing, using the one or more processors, a quality control procedure prior to obtaining the copy number value; and
   foregoing determining the LOH status of the one or more HLA-I genes in accordance with a determination that the sample is associated with a quality control issue.
40. The method of clause 39, wherein the quality control issue includes a sample contamination, a confirmed transplant, a noisy copy number, or a combination thereof.
41. The method of any of clauses 38 to 40, wherein the sample is at least one of a tumor tissue sample, a normal tissue sample, or a liquid biopsy sample.
42. The method of any of clauses 38 to 41, wherein the sample comprises one or more samples.
43. The method of any of clauses 38 to 42, wherein the predetermined range comprises a tumor purity between 20-95%.
44. The method of any of clauses 38 to 43, wherein the LOH status is determined without baiting the one or more HLA-I genes.
45. The method of any of clauses 38 to 44, wherein the one or more HLA-I genes comprise an HLA-A gene, an HLA-B gene, an HLA-C gene, or a combination thereof.
46. The method of any of clauses 38 to 45, wherein determining the loss of heterozygosity (LOH) status comprises:
   determining the LOH status to be indeterminate if the copy number value is at about a first threshold;
   determining the LOH status to be positive if the copy number value is at about a second threshold; and
   determining the LOH status to be negative if the copy number value is above a third threshold.
47. The method of clause 46, wherein:
   the first threshold is about 0,
   the second threshold is about 1, and
   the third threshold is about 2.
48. The method of any of clauses 46 to 47, further comprising:
   when the copy number value is at the third threshold, determining, using the one or more processors, whether the LOH status is a neutral LOH; and
   determining, using the one or more processors, the LOH status to be negative if the LOH status does not correspond to the neutral LOH and
   determining, using the one or more processors, the LOH status to be positive if the LOH status corresponds to the neutral LOH.
49. The method of any of clauses 38 to 48, wherein determining the copy number comprises:
   determining, using the one or more processors, a ploidy of the sample, coverage ratio data of the sample, allele fraction data of the sample, segmentation data of the sample, and a copy number model for the one or more HLA-I genes;
   determining, using the one or more processors, segment copy numbers for the one or more selected genomic segments that overlap with the one or more HLA-I genes in the sample based on at least the coverage ratio data, the allele fraction data, the segmentation data, and the copy number model;
   detecting, using the one or more processors, a presence of amplifications or deletions for one or more gene loci of the one or more HLA-I genes based on a segment copy number of the segment copy numbers for a corresponding genomic segment of the one or more selected genomic segments; and
   calling, using the one or more processors, the copy number of the one or more selected genomic segments based on the detected presence of amplifications or deletions for the one or more gene loci of the one or more HLA-I genes.
50. The method of clause 49, wherein determining the coverage ratio data comprises:
   aligning, using the one or more processors, a plurality of sequence reads of the one or more selected genomic segments that overlap with the one or more HLA-I genes in the sample to a reference genome;
   aligning, using the one or more processors, a plurality of sequence reads of one or more selected genomic segments that overlap with the one or more HLA-I genes in a control sample to the reference genome;
   determining, using the one or more processors, a number of sequence reads that overlap each of the one or more gene loci of the one or more HLA-I genes within the one or more selected genomic segments in the sample; and
   determining, using the one or more processors, a number of sequence reads that overlap each of the one or more gene loci of the one or more HLA-I genes within the one or more selected genomic segments in the control sample.
51. The method of any of clauses 49 to 50, wherein determining the allele fraction data comprises:
   aligning, using the one or more processors, a plurality of sequence reads of the one or more selected genomic segments that overlap with the one or more HLA-I genes in the sample in the sample to a reference genome;
   detecting a number of alleles present at a gene locus of the one or more gene loci of the one or more HLA-I genes; and
   determining an allele fraction for at least one of the alleles present at the gene locus.
52. The method of any of clauses 49 to 51, wherein determining the segmentation data comprises:
   aligning, using the one or more processors, a plurality of sequence reads of the one or more selected genomic segments that overlap with the one or more HLA-I genes in the sample to a reference genome;
   obtaining, using the one or more processors, aligned sequence read data based on the aligned plurality of sequence reads of the one or more selected genomic segments in the sample;
   processing, using the one or more processors, the aligned sequence read data of the sample, the coverage ratio data of the sample, and the allele fraction data of the sample using a pruned exact linear time (PELT) method; and
   determining a number of partial segments of the one or more selected genomic segments in the sample, wherein each partial segment has a same copy number.
53. The method any of clauses 38 to 52, wherein determining the copy number comprises:
   determining, using the one or more processors, normalized sequence read data and minor allele frequency (MAF) data based on the one or more selected genomic segments that overlap with the one or more HLA-I genes in the sample;
   segmenting, using the one or more processors, the normalized sequence read data using whole-genome segmentation;
   fitting, using the one or more processors, one or more copy number models to the segmented normalized sequence read data and the MAF data; and
   determining the copy number based on the fitted segmented normalized sequence read data and the MAF data.
54. The method of clause 53, wherein the genome segmentation comprises a circular binary segmentation algorithm, an HMM based method, a Wavelett based method, or a Cluster along Chromosomes method.
55. The method of any of clauses 53 to 54, further comprising segmenting, using the one or more processors, a genomic sequence of the one or more selected genomic sequences that overlap with the one or more HLA-I genes in the sample into partial genomic segments of equal copy number.
56. The method any of clauses 38 to 55, further comprising determining a zygosity score based on the copy number of the one or more selected genomic segments.
57. The method of clause 56, wherein a zygosity score of about 1 corresponds to a positive LOH status and a zygosity score of about 2 indicates a non-positive LOH status.
58. The method of any of clauses 56 to 57, wherein determining the zygosity score comprises:
   determining, using the one or more processors, a sequence coverage input (SCI), based on a number of reads of a selected genomic segment of the one or more selected genomic segments that overlap the one or more HLA-I genes in the sample;
   determining, using the one or more processors, a single nucleotide polymorphism (SNP) allele frequency input (SAFI), based on a SNP allele frequency for each of a plurality of selected germline SNPs in the sample; and
   determining, using the one or more processors, a variant allele frequency input (VAFI), based on an allele frequency for a variant associated with the LOH status;
      obtaining, using the one or more processors, values based on the SCI and the SAFI for:
      a genomic segment total copy number (C value), for each of the one or more selected genomic segments in the sample;
      a genomic segment minor allele copy number (M value), for each of the one or more selected genomic segments in the sample; and
      sample purity (p); and
      obtaining, using the one or more processors, the zygosity score as a function of the C value and the M value.
59. The method of clause 58, wherein a genomic segment of the one or more selected genomic segments in the sample comprises a plurality of subgenomic intervals, and wherein determining the zygosity score further comprises assigning, using the one or more processors, SCI values to the plurality of subgenomic intervals.
60. The method of any of clauses 38 to 59, further comprising determining, using the one or more processors, a treatment decision for a subject based on the LOH status of the one or more HLA-I genes.
61. The method of clause 60, wherein if the LOH status of the one or more HLA-I genes is a non-positive LOH status, the treatment comprises an immune checkpoint inhibitor (ICI).
62. The method of any of clauses 60 to 61, wherein if the LOH status of the one or more HLA-I genes is a positive LOH status, the treatment comprises a chemotherapy treatment, a non-ICI targeted treatment, a radiation therapy, a hormone therapy, or a combination thereof.
63. The method of any of clauses 38 to 62, further comprising administering, using the one or more processors, a treatment to a subject based on the LOH status of the one or more HLA-I genes.
64. The method of clause 63, wherein if the LOH status of the one or more HLA-I genes is a non-positive LOH status, the treatment comprises an immune checkpoint inhibitor (ICI) treatment.
65. The method of any of clauses 63 to 64, wherein if the LOH status of the one or more HLA-I genes is a positive LOH status, the treatment comprises a chemotherapy treatment, a non-ICI targeted treatment, a radiation therapy, a hormone therapy, or a combination thereof.
66. The method of any of clauses 38 to 65, further comprising assessing, using the one or more processors, an immunotherapy resistance of a subject based on the LOH status of the one or more HLA-I genes.
67. The method of any of clauses 38 to 66, further comprising monitoring, using the one or more processors, an immunotherapy resistance of a subject based on the LOH status of the one or more HLA-I genes over time.
68. The method any of clauses 38 to 67, further comprising predicting, using the one or more processors, one or more clinical outcomes based on the LOH status of the one or more HLA-I genes.
69. The method of any of clauses 38 to 68, further comprising predicting, using the one or more processors, an overall survival of the subject based on the LOH status of the one or more HLA-I genes.
70. A method for diagnosing a disease, the method comprising diagnosing that a subject has the disease based on a determination of a loss of heterozygosity (LOH) status for a sample from the subject, wherein the LOH status is determined according to the method of any one of clauses 38 to 69.
71. A method of selecting an anti-cancer therapy, the method comprising responsive to determining a loss of heterozygosity (LOH) status for a sample from a subject, selecting an anti-cancer therapy for the subject, wherein the LOH status is determined according to the method of any one of clauses 38 to 69.
72. A method of treating a cancer in a subject, comprising responsive to determining a loss of heterozygosity (LOH) for a sample from the subject, administering an effective amount of an anti-cancer therapy to the subject, wherein the LOH status is determined according to the method of any one of clauses 38 to 69.
73. A method for monitoring cancer progression or recurrence in a subject, the method comprising:
   determining a first loss of heterozygosity (LOH) status in a first sample obtained from the subject at a first time point according to the method of any one of clauses 38 to 69;
   determining a second LOH status in a second sample obtained from the subject at a second time point; and
   comparing the first LOH status to the second LOH status, thereby monitoring the cancer progression or recurrence.
74. The method of any one of clauses 38 to 69, further comprising determining, identifying, or applying a value of a loss of heterozygosity (LOH) status for the sample as a diagnostic value associated with the sample.
75. The method of any one of clauses 38 to 69, wherein the determination of a loss of heterozygosity (LOH) status for the sample is used in making suggested treatment decisions for the subject.
76. A system comprising:
   one or more processors; and
   a memory communicatively coupled to the one or more processors and configured to store instructions that, when executed by the one or more processors, cause the system to:
      identify, using the one or more processors, a plurality of genomic segments in a sample;
      select, using the one or more processors, from the plurality of genomic segments, one or more genomic segments that overlap with one or more HLA-I genes in the sample;
      determine, using the one or more processors, whether a tumor purity of the sample is in a predetermined range;
      when the tumor purity of the sample is in the predetermined range, obtain, using the one or more processors, a copy number value of the one or more selected genomic segments;
      determine, using the one or more processors, a loss of heterozygosity (LOH) status of the one or more HLA-I genes based on the copy number value.
77. A non-transitory computer-readable storage medium storing one or more programs, the one or more programs comprising instructions, which when executed by one or more processors of a system, cause the system to:
   identify, using the one or more processors, a plurality of genomic segments in a sample;
   select, using the one or more processors, from the plurality of genomic segments, one or more genomic segments that overlap with one or more HLA-I genes in the sample;
   determine, using the one or more processors, whether a tumor purity of the sample is in a predetermined range;
   when the tumor purity of the sample is in the predetermined range, obtain, using the one or more processors, a copy number value of the one or more selected genomic segments; and
   determine, using the one or more processors, a loss of heterozygosity (LOH) status of the one or more HLA-I genes based on the copy number value.
78. A method, comprising:
   identifying, using one or more processors, a plurality of genomic segments in a sample;
   selecting, using the one or more processors, from the plurality of genomic segments, one or more genomic segments that overlap with one or more HLA-I genes in the sample without baiting the one or more HLA-I genes;
determining, using the one or more processors, whether a tumor purity of the sample is in a predetermined range;
   when the tumor purity of the sample is in the predetermined range, obtaining, using the one or more processors, a copy number value of the one or more selected genomic segments;
   determining, using the one or more processors, a loss of heterozygosity (LOH) status of the one or more HLA-I genes.
79. A method comprising:
   providing a plurality of nucleic acid molecules obtained from a sample from a subject;
   ligating one or more adapters onto one or more nucleic acid molecules from the plurality of nucleic acid molecules;
   amplifying the one or more ligated nucleic acid molecules from the plurality of nucleic acid molecules;
   capturing amplified nucleic acid molecules from the amplified nucleic acid molecules;
   sequencing, by a sequencer, the captured nucleic acid molecules to obtain a plurality of sequence reads that represent the captured nucleic acid molecules;
   receiving, at one or more processors, sequence read data for the plurality of sequence reads, wherein one or more sequence reads of the plurality of sequence reads correspond to a plurality of genomic segments in the sample, wherein the one or more sequence reads are aligned with a reference sequence;
   determining, using the one or more processors:
      whether a number of sequence reads in the one or more sequence reads exceeds a predetermined sequence read threshold;
      whether the plurality of genomic segments are germline heterozygous at one or more HLA-I gene loci in the sample; and
      whether a tumor content of the sample is above a predetermined tumor content threshold; and
   when the plurality of genomic segments are germline heterozygous, the number of sequence reads in the one or more sequence reads exceeds the predetermined sequence read threshold, and the tumor content is above the predetermined tumor content threshold:
   determining, using the one or more processors, a gene minor allele frequency (MAF) of the sample and a segment MAF of the sample; and
   determining, using the one or more processors, a loss of heterozygosity (LOH) status based on the gene MAF of the sample, the segment MAF of the sample, and the tumor content of the sample.
80. The method of clause 79, wherein the tumor content corresponds to a maximum somatic allele frequency (MSAF).
81. The method of any of clauses 79 to 80, wherein the tumor content corresponds to a tumor purity.
82. The method of any of clauses 79 to 81, wherein the one or more HLA-I genes comprise an HLA-A gene, an HLA-B gene, an HLA-C gene, or a combination thereof.
83. The method of any of clauses 79 to 82, further comprising sequencing the one or more sequence reads corresponding to the plurality of genomic segments in the sample that have been baited for the one or more HLA-I genes.
84. The method of any of clauses 79 to 83, further comprising:
   aligning, using the one or more processors, the one or more sequence reads corresponding to a plurality of genomic segments in the sample to the reference sequence, the reference sequence associated with the one or more HLA-I gene loci.
85. The method of any of clauses 79 to 84, further comprising obtaining the sample from the subject.
86. The method of any of clauses 79 to 85, wherein determining whether the plurality of genomic segments in the sample are germline heterozygous at the one or more HLA-I genes in the sample comprises determining, using the one or more processors, an allele fraction of the sample at the one or more HLA-I gene loci is about 0.5.
87. The method of any of clauses 79 to 86, wherein determining whether the one or more sequence reads derived from the sample exceeds the predetermined sequence read threshold comprises:
   aligning, using the one or more processors, the one or more sequence reads corresponding to the plurality of genomic segments in the sample to the reference sequence, the reference sequence comprising one or more sequences for an HLA allele of the one or more HLA-I genes;
   determining, using the one or more processors, the number of sequence reads in the one or more sequence reads aligned with the reference sequence at the one or more HLA-I gene loci; and
   comparing, using the one or more processors, the number of sequence reads to the predetermined sequence read threshold.
88. The method of any of clauses 79 to 87, wherein the sequence read threshold is one of 1000 reads, 1100 reads, 1200 reads, 1300 reads, 1400 reads, 1500 reads, 1600 reads, 1700 reads, 1800 reads, 1900 reads, 2000 reads, 2100 reads, 2200 reads, 2300 reads, 2400 reads, 2500 reads, 2600 reads, 2700 reads, 2800 reads, 2900 reads, or 3000 reads.
89. The method of any of clauses 79 to 88, wherein the tumor content threshold corresponds to a tumor content of 1%, 5%, or 10%.
90. The method of any of clauses 79 to 89, wherein determining the second estimate of the tumor content of the sample based on the allele frequency determination comprises:
   determining whether a quality metric for the plurality of values is greater than a second threshold;
   based on a determination that the quality metric for the plurality of values is greater than the second threshold, determining the second estimate for the tumor content of the sample based on a first determination of somatic allele frequency, and
   based on a determination that the quality metric for the plurality of values is less than or equal to the second threshold, determining the second estimate for the tumor content of the sample based on a second determination of somatic allele frequency.
91. The method of any of clauses 79 to 90, wherein the tumor content is a value indicative of a ratio of circulating tumor DNA (ctDNA) to total cell-free DNA (cfDNA) in the sample.
92. The method of any of clauses 79 to 91, wherein determining the second estimate of the tumor content of the sample based on the allele frequency determination comprises:
   determining whether a quality metric for the plurality of values is greater than a second threshold;
   based on a determination that the quality metric for the plurality of values is greater than the second threshold, determining the second estimate for the tumor content of the sample based on a first determination of somatic allele frequency; and
   based on a determination that the quality metric for the plurality of values is less than or equal to the second threshold, determining the second estimate for the tumor content of the sample based on a second determination of somatic allele frequency.
93. The method of any one of clauses 79 to 92, wherein the subject is suspected of having or is determined to have cancer.
94. The method of clause 93, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenie carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, bead and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
95. The method of clause 93, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.
96. The method of any of clauses 93 to 95, further comprising treating the subject with an anti-cancer therapy.
97. The method of clause 96, wherein the anti-cancer therapy comprises a targeted anti-cancer therapy
98. The method of clause 97, wherein the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enbertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.
99. The method of any one of clauses 79 to 98, wherein the sample comprises one or more samples.
100. The method of any of clauses 79 to 99, wherein the sample comprises a normal tissue sample, a tumor tissue sample, or a liquid biopsy sample.
101. The method of clause 100, wherein the sample is a liquid biopsy sample and comprises blood, plasma, cerebrospinal fluid, sputum, stool, urine, or saliva.
102. The method of clause 100, wherein the sample is a liquid biopsy sample and comprises circulating tumor cells (CTCs).
103. The method of clause 100, wherein the sample is a liquid biopsy sample and comprises cell-free DNA (cfDNA), circulating tumor DNA (etDNA), or any combination thereof.
104. The method of any one of clauses 79 to 103, wherein the plurality of nucleic acid molecules comprises a mixture of tumor nucleic acid molecules and non-tumor nucleic acid molecules.
105. The method of clause 104, wherein the tumor nucleic acid molecules are derived from a tumor portion of a heterogeneous tissue biopsy sample, and the non-tumor nucleic acid molecules are derived from a normal portion of the heterogeneous tissue biopsy sample.
106. The method of embodiment 104, wherein the sample comprises a liquid biopsy sample, and wherein the tumor nucleic acid molecules are derived from a circulating tumor DNA (ctDNA) fraction of the liquid sample, and the non-tumor nucleic acid molecules are derived from a non-tumor, cell-free DNA (cfDNA) fraction of the liquid sample.
107. The method of any of clauses 79 to 106, wherein the one or more adapters comprise amplification primers, flow cell adaptor sequences, substrate adapter sequences, or sample index sequences.
108. The method of any of clauses 79 to 107, wherein the captured nucleic acid molecules are captured from the amplified nucleic acid molecules by hybridization to one or more bait molecules.
109. The method of clause 108, wherein the one or more bait molecules comprise one or more nucleic acid molecules, each comprising a region that is complementary to a region of a captured nucleic acid molecule.
110. The method of one of clauses 79 to 109, wherein amplifying nucleic acid molecules comprises performing a polymerase chain reaction (PCR) amplification technique, a non-PCR amplification technique, or an isothermal amplification technique.
111. The method of any of clauses 79 to 110, wherein the sequencing comprises use of a massively parallel sequencing (MPS) technique, whole genome sequencing (WGS), whole exome sequencing, targeted sequencing, direct sequencing, or Sanger sequencing technique.
112. The method of clause 111, wherein the sequencing comprises massively parallel sequencing, and the massively parallel sequencing technique comprises next generation sequencing (NGS).
113. The method of any one of clauses 79 to 112, wherein the sequencer comprises a next generation sequencer.
114. The method of any one of clauses 79 to 113, wherein the one or more sequence reads overlap one or more gene loci within one or more subgenomic intervals in the sample.
115. The method of clause 114, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.
116. The method of clause 114 or clause 115, wherein the one or more gene loci comprise ABL1, ACVR1B, AKTI, AKT2, AKT3, ALK, ALOX12B, AMER1, APC, AR, ARAF, ARFRP1, ARIDIA, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKNIB, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBPI, GABRA6, GATA3, GATA4, GATA6, GID4 (C17orf39), GNA11, GNA13, GNAQ, GNAS, GRM3, GSK3B, H3F3A, HDAC1, HGF, HNFIA, HRAS, HSD3B1, ID3, IDH1, IDH2, IGFIR, IKBKE, IKZF1, INPP4B, IRF2, IRF4, IRS2, JAKI, JAK2, JAK3, JUN, KDM5A, KDM5C, KDM6A, KDR, KEAPI, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPKI, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKB1A, NKX2-1, NOTCH1, NOTCH2, NOTCH3, NPM1, NRAS, NT5C2, NTRK1, NTRK2, NTRK3, NUTMI, P2RY8, PALB2, PARK2, PARP1, PARP2, PARP3, PAX5, PBRM1, PDCD1, PDCDILG2, PDGFRA, PDGFRβ, PDK1, PIK3C2B, PIK3C2G, PIK3C_{A}, PIK3C_{B}, PIK3R1, PIM1, PMS2, POLD1, POLE, PPARG, PPP2R1A, PPP2R2A, PRDM1, PRKARIA, PRKCI, PTCH1, PTEN, PTPN11, PTPRO, QKI, RACI, RAD21, RAD51, RAD51B, RAD51C, RAD51D, RAD52, RAD54L, RAF1, RARA, R_{B}1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCIL1, WTI, XPO1, XRCC2, ZNF217, ZNF703, or any combination thereof.
117. The method of clause 114 or clause 115, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFR&, PDGFRβ, PD-L1, PI3Kδ, PIGF, PTCH, RAF, RANKL, RET, ROS1, SLAMF7, VEGF, VEGFA, VEGFB, or any combination thereof.
118. The method of any one of clauses 79 to 117, further comprising generating, by the one or more processors, a report indicating the LOH status of the one or more HLA-I genes.
119. The method of clause 118, further comprising transmitting the report to a healthcare provider.
120. The method of clause 119, wherein the report is transmitted via a computer network or a peer-to-peer connection.
121. A method, comprising:
   receiving, at one or more processors, one or more sequence reads corresponding to a plurality of genomic segments in a sample from a subject, wherein the one or more sequence reads are aligned with a reference sequence;
   determining, using the one or more processors:
      whether a number of sequence reads in the one or more sequence reads exceeds a predetermined sequence read threshold;
      whether the plurality of genomic segments are germline heterozygous at one or more HLA-I gene loci in the sample; and
      whether a tumor content of the sample is above a predetermined tumor content threshold;
   when the plurality of genomic segments are germline heterozygous, the number of sequence reads in the one or more sequence reads exceeds the predetermined sequence read threshold, and the tumor content is above the predetermined tumor content threshold:
      determining, using the one or more processors, a gene minor allele frequency (MAF) of the sample and a segment MAF of the sample; and
      determining, using the one or more processors, a loss of heterozygosity (LOH) status based on the gene MAF of the sample, the segment MAF of the sample, and the tumor content of the sample.
122. The method of clause 121, wherein the sample comprises at least one of a tissue sample, a liquid biopsy sample, or a normal tissue sample.
123. The method of any of clauses 121 to 122, wherein the tumor content corresponds to a maximum somatic allele frequency (MSAF), a tumor purity, or a combination thereof.
124. The method of any of clauses 121 to 123, wherein the sample comprises one or more samples.
125. The method of any of clauses 121 to 124, wherein the one or more HLA-I genes comprise an HLA-A gene, an HLA-B gene, an HLA-C gene, or a combination thereof.
126. The method of any of clauses 121 to 125, further comprising sequencing, using the one or more processors, the one or more reads corresponding to the plurality of genomic segments in the sample.
127. The method of any of clauses 121 to 126, further comprising:
   aligning, using the one or more processors, the one or more reads corresponding to a plurality of genomic segments in the sample to the reference sequence.
128. The method of any of clauses 121 to 127, wherein determining whether the plurality of genomic segments in the sample are germline heterozygous at the one or more HLA-I gene loci in the sample comprises determining, using the one or more processors, an allele fraction of the sample at the one or more HLA-I gene loci is about 0.5.
129. The method of any of clauses 121 to 128, wherein determining whether the one or more sequence reads derived from the sample exceeds the predetermined sequence read threshold comprises:
   aligning, using the one or more processors, the one or more sequence reads corresponding to the plurality of genomic segments in the sample to the reference sequence, the reference sequence comprising one or more sequences for an HLA allele of the one or more HLA-I genes; and
   determining, using the one or more processors, the number of sequence reads in the one or more sequence reads aligned with the reference sequence at the one or more HLA-I gene loci; and
   comparing, using the one or more processors, the number of sequence reads to the predetermined sequence read threshold.
130. The method of any of clauses 121 to 129, wherein the sequence read threshold is one of 1000 reads, 1100 reads, 1200 reads, 1300 reads, 1400 reads, 1500 reads, 1600 reads, 1700 reads, 1800 reads, 1900 reads, 2000 reads, 2100 reads, 2200 reads, 2300 reads, 2400 reads, 2500 reads, 2600 reads, 2700 reads, 2800 reads, 2900 reads, or 3000 reads.
131. The method of any of clauses 121 to 130, wherein the tumor content threshold corresponds to a tumor content of 1%, 5%, or 10%.
132. The method of any of clauses 121 to 123 and 125 to 131, wherein determining whether the tumor content of the sample is above a predetermined tumor content threshold comprises:
   receiving, at the one or more processors, a plurality of values, each value indicative of an allele fraction at a gene locus within the plurality of genomic segments in the sample;
   determining, by the one or more processors, a certainty metric value indicative of a dispersion of the plurality of values;
   determining, by the one or more processors, a first estimate of the tumor content of the sample, the first estimate based on the certainty metric value for the sample and a predetermined relationship between one or more stored certainty metric values and one or more stored tumor fraction values;
   determining, by the one or more processors, whether a value associated with the first estimate is greater than a first threshold;
   based on a determination that the value associated with the first estimate is greater than the first threshold, outputting, by the one or more processors, the first estimate as the tumor content of the sample; and
   based on a determination that the value associated with the first estimate is less than or equal to the first threshold:
   determining, by the one or more processors, a second estimate of the tumor content of the sample;
   outputting, by the one or more processors, the second estimate as the tumor content of the sample; and
   comparing the tumor content of the sample to the tumor content threshold.
133. The method of clause 132, wherein the tumor content is a value indicative of a ratio of circulating tumor DNA (ctDNA) to total cell-free DNA (cfDNA) in the sample.
134. The method of any of clauses 132 to 133, wherein determining the second estimate of the tumor content of the sample based on the allele frequency determination comprises:
   determining whether a quality metric for the plurality of values is greater than a second threshold;
   based on a determination that the quality metric for the plurality of values is greater than the second threshold, determining the second estimate for the tumor content of the sample based on a first determination of somatic allele frequency, and
   based on a determination that the quality metric for the plurality of values is less than or equal to the second threshold, determining the second estimate for the tumor content of the sample based on a second determination of somatic allele frequency.
135. The method of any of clauses 121 to 134, further comprising training a model for determining the LOH status of a sample, the training comprising:
   receiving, at the one or more processors, one or more reads corresponding to one or more selected genomic segments in a paired sample, wherein the one or more reads corresponding to the one or more selected genomic segments are aligned with the reference sequence;
   for each paired sample:
      fitting, using the one or more processors, one or more values associated with the one or more reads corresponding to the one or more selected genomic segments to a model;
      determining, using the one or more processors, a LOH status of the corresponding paired sample based on the fitted model; and
   determining, using the one or more processors, the predetermined sequence read threshold and the tumor content threshold based on the fitted model and the LOH status for the one or more selected genomic segments in the paired sample.
136. The method of any of clauses 121 to 135, further comprising determining, using the one or more processors, a treatment decision for a subject based on the LOH status.
137. The method of clause 136, wherein if the LOH status is a non-positive LOH status, the treatment comprises an immune checkpoint inhibitor (ICI).
138. The method of any of clauses 121 to 137, wherein if the LOH status is a positive LOH status, the treatment comprises a chemotherapy treatment, a non-ICI targeted treatment, radiation therapy, hormone therapy, or a combination thereof.
139. The method of any of clauses 121 to 138, further comprising administering, using the one or more processors, a treatment to a subject based on the LOH status.
140. The method of clause 139, wherein if the LOH status is a non-positive LOH status, the treatment comprises an immune checkpoint inhibitor (ICI) treatment.
141. The method of any of clauses 121 to 140, wherein if the LOH status is a positive LOH status, the treatment comprises a chemotherapy treatment, a non-ICI targeted treatment, a radiation therapy, a hormone therapy, or a combination thereof.
142. The method of any of clauses 121 to 141, further comprising assessing, using the one or more processors, an immunotherapy resistance of a subject based on the LOH status.
143. The method of any of clauses 121 to 142, further comprising monitoring, using the one or more processors, an immunotherapy resistance of a subject based on the LOH status over time.
144. The method any of clauses 121 to 143, further comprising predicting, using the one or more processors, one or more clinical outcomes based on the LOH status.
145. The method of any of clauses 121 to 144, further comprising predicting, using the one or more processors, an overall survival of the subject based on the LOH status.
146. A method for diagnosing a disease, the method comprising diagnosing that a subject has the disease based on a determination of a loss of heterozygosity (LOH) status for a sample from the subject, wherein the LOH status is determined according to the method of any one of clauses 121 to 145.
147. A method of selecting an anti-cancer therapy, the method comprising responsive to determining a loss of heterozygosity (LOH) status for a sample from a subject, selecting an anti-cancer therapy for the subject, wherein the LOH status is determined according to the method of any one of clauses 121 to 145.
148. A method of treating a cancer in a subject, comprising responsive to determining a loss of heterozygosity (LOH) for a sample from the subject, administering an effective amount of an anti-cancer therapy to the subject, wherein the LOH status is determined according to the method of any one of clauses 121 to 145.
149. A method for monitoring cancer progression or recurrence in a subject, the method comprising:
   determining a first loss of heterozygosity (LOH) status in a first sample obtained from the subject at a first time point according to the method of any one of clauses 121 to 145;
   determining a second LOH status in a second sample obtained from the subject at a second time point; and comparing the first LOH status to the second LOH status, thereby monitoring the cancer progression or recurrence.
150. The method of any one of clauses 121 to 145, further comprising determining, identifying, or applying the value of a loss of heterozygosity (LOH) status for the sample as a diagnostic value associated with the sample.
151. The method of any one of clauses 121 to 145, wherein the determination of a loss of heterozygosity (LOH) status for the sample is used in making suggested treatment decisions for the subject.
152. A system comprising:
   one or more processors; and
   a memory communicatively coupled to the one or more processors and configured to store instructions that, when executed by the one or more processors, cause the system to:
      receive, at the one or more processors, one or more sequence reads corresponding to a plurality of genomic segments in a sample from a subject, wherein the one or more sequence reads are aligned with a reference sequence;
      determine, using the one or more processors:
         whether a number of sequence reads in the one or more sequence reads exceeds a predetermined sequence read threshold;
         whether the plurality of genomic segments are germline heterozygous at one or more HLA-I gene loci in the sample; and
         whether a tumor content of the sample is above a predetermined tumor content threshold; and
      when the plurality of genomic segments are germline heterozygous, the number of sequence reads of one or more sequence reads exceeds the predetermined sequence read threshold, and the tumor content is above the predetermined tumor content threshold:
      determine, using the one or more processors, a gene minor allele frequency (MAF) of the sample and a segment MAF of the sample; and
      determine, using the one or more processors, a loss of heterozygosity (LOH) status of the sample based on the gene MAF of the sample, the segment MAF of the sample, and the tumor content of the sample.
153. A non-transitory computer-readable storage medium storing one or more programs, the one or more programs comprising instructions, which when executed by one or more processors of a system, cause the system to:
   receive, at the one or more processors, one or more sequence reads corresponding to a plurality of genomic segments in a sample from a subject, wherein the one or more sequence reads are aligned with a reference sequence;
   determine, using the one or more processors:
      whether a number of sequence reads in the one or more sequence reads exceeds a predetermined sequence read threshold;
      whether the plurality of genomic segments are germline heterozygous at one or more HLA-I gene loci in the sample; and
      whether a tumor content of the sample is above a predetermined tumor content threshold;
   when the plurality of genomic segments are germline heterozygous, the number of sequence reads of one or more sequence reads exceeds the predetermined sequence read threshold, and the tumor content is above the predetermined tumor content threshold:
      determine, using the one or more processors, a gene minor allele frequency (MAF) of the sample and a segment MAF of the sample; and
      determine, using the one or more processors, a loss of heterozygosity (LOH) status of the sample based on the gene MAF of the sample, the segment MAF of the sample, and the tumor content of the sample.
154. A method of treating a subject having a cancer comprising:
   identifying a plurality of genomic segments in a sample;
   selecting from the plurality of genomic segments, one or more genomic segments that overlap with one or more HLA-I genes in the sample;
   determining whether a tumor purity of the sample is in a predetermined range;
   when the tumor purity of the sample is in the predetermined range, obtaining a copy number value of the one or more selected genomic segments;
   determining a loss of heterozygosity (LOH) status of the one or more HLA-I genes based on the copy number value, wherein an HLA-I LOH positive status is determined if the copy number value is associated with a first threshold; and
   treating the subject with a non-immune-oncology (IO) therapy if the subject is determined to have the HLA-I LOH positive status; and
   treating the subject with an IO therapy if the subject is determined to have an HLA-I LOH non-positive status.
155. A method of selecting a treatment for a subject having a cancer comprising:
   identifying a plurality of genomic segments in a sample;
   selecting from the plurality of genomic segments, one or more genomic segments that overlap with one or more HLA-I genes in the sample;
   determining whether a tumor purity of the sample is in a predetermined range;
   when the tumor purity of the sample is in the predetermined range, obtaining a copy number value of the one or more selected genomic segments;
   determining a loss of heterozygosity (LOH) status of the one or more HLA-I genes based on the copy number value, wherein an HLA-I LOH positive status is determined if the copy number value is associated with a first threshold; and
   identifying the subject for treatment with a non-IO therapy if the subject is determined to have the HLA-I LOH positive status; and
   identifying the subject for treatment with a IO therapy if the subject is determined to have an HLA-I LOH non-positive status.
156. A method of identifying a subject having a cancer for treatment with an immune oncology (IO) therapy, the method comprising:
   identifying a plurality of genomic segments in a sample;
   selecting from the plurality of genomic segments, one or more genomic segments that overlap with one or more HLA-I genes in the sample;
   determining whether a tumor purity of the sample is in a predetermined range;
   when the tumor purity of the sample is in the predetermined range, obtaining a copy number value of the one or more selected genomic segments;
   determining a loss of heterozygosity (LOH) status of the one or more HLA-I genes based on the copy number value, wherein an HLA-I LOH positive status is determined if the copy number value is associated with a first threshold; and
   identifying the subject for treatment with an immune-oncology (IO) therapy if the subject is determined to have an HLA-I LOH non-positive status; and
   identifying the subject for treatment with an non-IO therapy if the subject is determined to have the HLA-I LOH positive status.
157. A method of identifying one or more treatment options for a subject having a cancer, the method comprising:
   identifying a plurality of genomic segments in a sample;
   selecting from the plurality of genomic segments, one or more genomic segments that overlap with one or more HLA-I genes in the sample;
   determining whether a tumor purity of the sample is in a predetermined range;
   when the tumor purity of the sample is in the predetermined range, obtaining a copy number value of the one or more selected genomic segments;
   determining a loss of heterozygosity (LOH) status of the one or more HLA-I genes based on the copy number value, wherein an HLA-I LOH positive status is determined if the copy number value is associated with a first threshold; and
   generating a report comprising one or more treatment options identified for the subject based at least in part on the HLA-I LOH status determined for the sample, wherein:
      the subject is identified as one who may benefit from treatment with an immuno-oncology (IO) therapy if the subject is determined to have an HLA-I LOH non-positive status, and
the subject is identified as one who may benefit from treatment with a non-IO therapy if the subject is determined to have the HLA-I LOH positive status.
158. A method of stratifying a subject having cancer for treatment with an immuno-oncology (IO) therapy, the method comprising:
   identifying a plurality of genomic segments in a sample;
   selecting from the plurality of genomic segments, one or more genomic segments that overlap with one or more HLA-I genes in the sample;
   determining whether a tumor purity of the sample is in a predetermined range;
   when the tumor purity of the sample is in the predetermined range, obtaining a copy number value of the one or more selected genomic segments;
   determining a loss of heterozygosity (LOH) status of the one or more HLA-I genes based on the copy number value, wherein an HLA-I LOH positive status is determined if the copy number value is associated with a first threshold; and
   treating the subject with the IO therapy if the subject is determined to have an HLA-I LOH non-positive status; and
   treating the subject with a non-IO therapy if the subject is determined to have the HLA-I LOH positive status.
159. A method of predicting survival of a subject having cancer, the method comprising:
   identifying a plurality of genomic segments in a sample;
   selecting from the plurality of genomic segments, one or more genomic segments that overlap with one or more HLA-I genes in the sample;
   determining whether a tumor purity of the sample is in a predetermined range;
   when the tumor purity of the sample is in the predetermined range, obtaining a copy number value of the one or more selected genomic segments;
   determining a loss of heterozygosity (LOH) status of the one or more HLA-I genes based on the copy number value, wherein an HLA-I LOH positive status is determined if the copy number value is associated with a first threshold; and
   if the subject is determined to have an HLA-I LOH non-positive status, the subject is predicted to have longer survival when treated with an immuno-oncology (IO) therapy, as compared to a subject that was determined to have the HLA-I LOH positive status; and
   if the subject is determined to have the HLA-I LOH positive status, the subject is predicted to have shorter survival when treated with an IO therapy, as compared to a subject that was determined to have the HLA-I LOH non-positive status.
160. A method of predicting survival of a subject having cancer, the method comprising:
   identifying a plurality of genomic segments in a sample;
   selecting from the plurality of genomic segments, one or more genomic segments that overlap with one or more HLA-I genes in the sample;
   determining whether a tumor purity of the sample is in a predetermined range;
   when the tumor purity of the sample is in the predetermined range, obtaining a copy number value of the one or more selected genomic segments; and
   determining a loss of heterozygosity (LOH) status of the one or more HLA-I genes based on the copy number value, wherein an HLA-I LOH positive status is determined if the copy number value is associated with a first threshold;
   wherein if the subject is determined to have the HLA-I LOH positive status, the subject is predicted to have longer survival when treated with a non-immuno-oncology (IO) therapy, as compared to a subject that was treated with an IO therapy.
161. The method of any one of clause 159 or clause 160, further comprising determining a tumor mutational burden (TMB) in the sample from the subject, wherein the predicted survival is further based on the TMB.
162. The method of clause 161, wherein the subject is determined to have a TMB of at least about 4 to 100 mutations/Mb, about 4 to 30 mutations/Mb, 8 to 100 mutations/Mb, 8 to 30 mutations/Mb, 10 to 20 mutations/Mb, less than 4 mutations/Mb, or less than 8 mutations/Mb.
163. The method of clause 162, wherein the TMB is at least about 5 mutations/Mb, is at least about 10 mutations/Mb, at least about 12 mutations/Mb, at least about 16 mutations/Mb, at least about 20 mutations/Mb, or at least about 30 mutations/Mb.
164. The method of clause 161 or clause 162, wherein the TMB is determined based on between about 100 kb to about 10 Mb of genomic sequence.
165. The method of clause 161 or clause 162, wherein the TMB is determined based on between about 0.8 Mb to about 1.1 Mb of genomic sequence.
166. The method of any one of clauses 154-165, further comprising treating the subject determined to have an HLA-I LOH non-positive status with an IO therapy.
167. The method of clause 166, wherein the IO therapy comprises a single IO agent or multiple IO agents.
168. The method of any one of clause 166 or clause 167, wherein the IO therapy comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a cellular therapy, a treatment for cancer being tested in a clinical trial, an immunotherapy, or any combination thereof.
169. The method of any one of clauses 166-168, wherein the IO therapy comprises an immune checkpoint inhibitor.
170. The method of clause 169, wherein the immune checkpoint inhibitor is a PD-1 inhibitor.
171. The method of clause 170, wherein the immune checkpoint inhibitor comprises one or more of nivolumab, pembrolizumab, cemiplimab, or dostarlimab.
172. The method of clause 169, wherein the immune checkpoint inhibitor is a PD-L1-inhibitor.
173. The method of clause 172, wherein the immune checkpoint inhibitor comprises one or more of atezolizumab, avelumab, or durvalumab.
174. The method of clause 169, wherein the immune checkpoint inhibitor is a CTLA-4 inhibitor.
175. The method of clause 174, wherein the CTLA-4 inhibitor comprises ipilimumab.
176. The method of clause 168, wherein the nucleic acid comprises a double-stranded RNA (dsRNA), a small interfering RNA (siRNA), or a small hairpin RNA (shRNA).
177. The method of clause 168, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.
178. The method of any one of clauses 1-177, wherein the non-IO therapy comprises one or more of a small molecule inhibitor, a chemotherapeutic agent, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.
179. The method of clause 178, wherein the chemotherapeutic agent comprises one or more of an alkylating agent, an alkyl sulfonates aziridine, an ethylenimine, a methylamelamine, an acetogenin, a camptothecin, a bryostatin, a callystatin, CC-1065, a cryptophycin, aa dolastatin, a duocarmycin, a eleutherobin, a pancratistatin, a sarcodictyin, a spongistatin, a nitrogen mustard, a nitrosureas, an antibiotic, a dynemicin, a bisphosphonate, an esperamicina a neocarzinostatin chromophore or a related chromoprotein enediyne antiobiotic chromophore, an anti-metabolite, a folic acid analogue, a purine analog, a pyrimidine analog, an androgens, an anti-adrenal, a folic acid replenisher, aldophosphamide glycoside, aminolevulinic acid, eniluracil, amsacrine, bestrabucil, bisantrene, edatraxate, defofamine, demecolcine, diaziquone, elformithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, a PSK polysaccharide complex, razoxane, rhizoxin, sizofiran, spirogermanium, tenuazonic acid, triaziquone, 2,2′,2″-trichlorotriethylamine, a trichothecene, urethan, vindesine, dacarbazine, mannomustine, mitobronitol, mitolactol, pipobroman, gacytosine, arabinoside (“Ara-C”), cyclophosphamide, a taxoid, 6-thioguanine, mercaptopurine, a platinum coordination complex, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluorometlhylomithine (DMFO), a retinoid, capecitabine, carboplatin, procarbazine, plicomycin, gemcitabine, navelbine, farnesyl-protein transferase inhibitors, transplatinum, or any combination thereof.
180. The method of any one of clauses 154-156 or 158-179, further comprising treating the subject determined to have an HLA-I LOH positive status with the non-IO therapy.
181. The method of any one of clauses 153-180, further comprising treating the subject with an additional anti-cancer therapy.
182. The method of clause 181, wherein the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.
183. The method of any one of clauses 159-179, wherein the survival is a progression-free survival, an overall survival, a disease-free survival (DFS), an objective response rate (ORR), a time to tumor progression (TTP), a time to treatment failure (TTF), a durable complete response (DCR), or a time to next treatment (TTNT).
184. The method of any one of clauses 154-183, wherein the sample comprises a tissue biopsy sample or a liquid biopsy sample.
185. The method of clause 184, wherein the sample is a tissue biopsy and comprises a tumor biopsy, tumor specimen, or circulating tumor cells.
186. The method of clause 184, wherein the sample is a liquid biopsy sample and comprises blood, serum, plasma, cerebrospinal fluid, sputum, stool, urine, or saliva.
187. The method of any one of clauses 154-186, wherein the sample comprises cells and/or nucleic acids from the cancer.
188. The method of clause 187, wherein the sample comprises mRNA, DNA, circulating tumor DNA (ctDNA), cell-free DNA, cell-free RNA from the cancer, or any combination thereof.
189. The method of clause 184, wherein the sample is a liquid biopsy sample and comprises circulating tumor cells (CTCs).
190. The method of clause 184, wherein the sample is a liquid biopsy sample and comprises cell-free DNA (cfDNA), circulating tumor DNA (ctDNA), or any combination thereof.
191. The method of any one of clauses 154-190, wherein the LOH status of the gene is determined based on sequence read data derived from sequencing nucleic acid molecules extracted from the sample from the subject.
192. The method of clause 191, wherein the sequencing comprises use of a massively parallel sequencing (MPS) technique, whole genome sequencing (WGS), whole exome sequencing, targeted sequencing, direct sequencing, next-generation sequencing (NGS), or a Sanger sequencing technique.
193. The method of clause 191 or clause 192, wherein the sequencing comprises:
   (a) providing a plurality of nucleic acid molecules obtained from the sample, wherein the plurality of nucleic acid molecules comprises a mixture of tumor nucleic acid molecules and non-tumor nucleic acid molecules;
   (b) optionally, ligating one or more adapters onto one or more nucleic acid molecules from the plurality of nucleic acid molecules;
   (c) amplifying nucleic acid molecules from the plurality of nucleic acid molecules;
   (d) optionally, capturing nucleic acid molecules from the amplified nucleic acid molecules, wherein the captured nucleic acid molecules are captured from the amplified nucleic acid molecules by hybridization to one or more bait molecules; and
   (e) sequencing, by a sequencer, the captured nucleic acid molecules to obtain a plurality of sequence reads corresponding to one or more genomic loci within a subgenomic interval in the sample.
194. The method of clause 193, wherein the adapters comprise one or more of amplification primer sequences, flow cell adapter hybridization sequences, unique molecular identifier sequences, substrate adapter sequences, or sample index sequences.
195. The method of clause 193 or clause 194, wherein amplifying nucleic acid molecules comprises performing a polymerase chain reaction (PCR) technique, a non-PCR amplification technique, or an isothermal amplification technique.
196. The method of any one of clauses 193-195, wherein the one or more bait molecules comprise one or more nucleic acid molecules, each comprising a region that is complementary to a region of a captured nucleic acid molecule.
197. The method of clause 196, wherein the one or more bait molecules each comprise a capture moiety.
198. The method of clause 197, wherein the capture moiety is biotin.
199. The method of any one of clauses 154-198, wherein the cancer is a B cell cancer, a melanoma, breast cancer, lung cancer, bronchus cancer, colorectal cancer or carcinoma, prostate cancer, pancreatic cancer, stomach cancer, ovarian cancer, urinaary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
200. The method of any of clauses 154-199, wherein the subject is a human.
201. The method of any one of clauses 154-200, wherein the subject has previously been treated with an anti-cancer therapy.
202. The method of clause 201, wherein the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.
203. A method of treating a subject having a cancer comprising:
   (a) receiving a plurality of sequence reads corresponding to a plurality of genomic segments in a sample from a subject, wherein the plurality of sequence reads are aligned with a reference sequence;
   (b) determining:
      (i) whether a number of sequence reads in the plurality of sequence reads exceeds a predetermined sequence read threshold;
      (ii) whether the plurality of genomic segments are germline heterozygous at one or more HLA-I gene loci in the sample; and
      (iii) whether a tumor content of the sample is above a predetermined tumor content threshold;
   (c) when the plurality of genomic segments are germline heterozygous, the number of sequence reads in the plurality of sequence reads exceeds the predetermined sequence read threshold, and the tumor content is above the predetermined tumor content threshold:
      (i) determining a gene minor allele frequency (MAF) of the sample and a segment MAF of the sample; and
      (ii) determining a loss of heterozygosity (LOH) status based on the gene MAF of the sample, the segment MAF of the sample, and the tumor content of the sample, wherein an HLA-I LOH positive status is determined if the tumor content is above the predetermined tumor content threshold and the gene MAF is below a first gene threshold and the segment MAF is below a first segment threshold;
   (d) treating the subject with a non-immune-oncology (10) therapy if the subject is determined to have the HLA-I LOH positive status; and
   (e) treating the subject with an IO therapy if the subject is determined to have an HLA-I LOH non-positive status.
204. A method of selecting a treatment for a subject having a cancer, the method comprising:
   (a) receiving a plurality of sequence reads corresponding to a plurality of genomic segments in a sample from a subject, wherein the plurality of sequence reads are aligned with a reference sequence;
   (b) determining:
      (i) whether a number of sequence reads in the plurality of sequence reads exceeds a predetermined sequence read threshold;
      (ii) whether the plurality of genomic segments are germline heterozygous at one or more HLA-I gene loci in the sample; and
      (iii) whether a tumor content of the sample is above a predetermined tumor content threshold;
   (c) when the plurality of genomic segments are germline heterozygous, the number of sequence reads in the plurality of sequence reads exceeds the predetermined sequence read threshold, and the tumor content is above the predetermined tumor content threshold:
      (i) determining a gene minor allele frequency (MAF) of the sample and a segment MAF of the sample; and
      (ii) determining a loss of heterozygosity (LOH) status based on the gene MAF of the sample, the segment MAF of the sample, and the tumor content of the sample, wherein an HLA-I LOH positive status is determined if the tumor content is above the predetermined tumor content threshold and the gene MAF is below a first gene threshold and the segment MAF is below a first segment threshold;
   (d) identifying the subject for treatment with a non-immune-oncology (IO) therapy if the subject is determined to have the HLA-I LOH positive status; and
   (e) identifying the subject for treatment with a IO therapy if the subject is determined to have an HLA-I LOH non-positive status.
205. A method of identifying a subject having a cancer for treatment with an immune oncology (IO) therapy, the method comprising:
   (a) receiving a plurality of sequence reads corresponding to a plurality of genomic segments in a sample from a subject, wherein the plurality of sequence reads are aligned with a reference sequence;
   (b) determining:
      (i) whether a number of sequence reads in the plurality of sequence reads exceeds a predetermined sequence read threshold;
      (ii) whether the plurality of genomic segments are germline heterozygous at one or more HLA-I gene loci in the sample; and
      (iii) whether a tumor content of the sample is above a predetermined tumor content threshold;
   (c) when the plurality of genomic segments are germline heterozygous, the number of sequence reads in the plurality of sequence reads exceeds the predetermined sequence read threshold, and the tumor content is above the predetermined tumor content threshold
      (i) determining a gene minor allele frequency (MAF) of the sample and a segment MAF of the sample; and
      (ii) determining a loss of heterozygosity (LOH) status based on the gene MAF of the sample, the segment MAF of the sample, and the tumor content of the sample, wherein an HLA-I LOH positive status is determined if the tumor content is above the predetermined tumor content threshold and the gene MAF is below a first gene threshold and the segment MAF is below a first segment threshold;
   (d) identifying the subject for treatment with a non-immune-oncology (IO) therapy if the subject is determined to have the HLA-I LOH positive status; and
   (e) identifying the subject for treatment with an IO therapy if the subject is determined to have an HLA-I LOH non-positive status.
206. A method of identifying one or more treatment options for a subject having a cancer, the method comprising:
   (a) receiving a plurality of sequence reads corresponding to a plurality of genomic segments in a sample from a subject, wherein the plurality of sequence reads are aligned with a reference sequence;
   (b) determining:
      (i) whether a number of sequence reads in the plurality of sequence reads exceeds a predetermined sequence read threshold;
      (ii) whether the plurality of genomic segments are germline heterozygous at one or more HLA-I gene loci in the sample; and
      (iii) whether a tumor content of the sample is above a predetermined tumor content threshold;
   (c) when the plurality of genomic segments are germline heterozygous, the number of sequence reads in the plurality of sequence reads exceeds the predetermined sequence read threshold, and the tumor content is above the predetermined tumor content threshold:
      (i) determining a gene minor allele frequency (MAF) of the sample and a segment MAF of the sample; and
      (ii) determining a loss of heterozygosity (LOH) status based on the gene MAF of the sample, the segment MAF of the sample, and the tumor content of the sample, wherein an HLA-I LOH positive status is determined if the tumor content is above the predetermined tumor content threshold and the gene MAF is below a first gene threshold and the segment MAF is below a first segment threshold; and
   (d) generating a report comprising one or more treatment options identified for the subject based at least in part on the HLA-I LOH status determined for the sample, wherein:
      (i) the subject is identified as one who may benefit from treatment with a non-immune-oncology (IO) therapy if the subject is determined to have the HLA-I LOH positive status; and
      (ii) the subject is identified as one who may benefit from treatment with an IO therapy if the subject is determined to have an HLA-I LOH non-positive status.
207. A method of stratifying a subject having cancer for treatment with an immuno-oncology (IO) therapy, the method comprising:
   (a) receiving a plurality of sequence reads corresponding to a plurality of genomic segments in a sample from a subject, wherein the plurality of sequence reads are aligned with a reference sequence;
   (b) determining:
      (i) whether a number of sequence reads in the plurality of sequence reads exceeds a predetermined sequence read threshold;
      (ii) whether the plurality of genomic segments are germline heterozygous at one or more HLA-I gene loci in the sample; and
      (iii) whether a tumor content of the sample is above a predetermined tumor content threshold;
   (c) when the plurality of genomic segments are germline heterozygous, the number of sequence reads in the plurality of sequence reads exceeds the predetermined sequence read threshold, and the tumor content is above the predetermined tumor content threshold:
      (i) determining a gene minor allele frequency (MAF) of the sample and a segment MAF of the sample; and
      (ii) determining a loss of heterozygosity (LOH) status based on the gene MAF of the sample, the segment MAF of the sample, and the tumor content of the sample, wherein an HLA-I LOH positive status is determined if the tumor content is above the predetermined tumor content threshold and the gene MAF is below a first gene threshold and the segment MAF is below a first segment threshold; and
   (d) treating the subject with the IO therapy if the subject is determined to have an HLA-I LOH non-positive status; and
   (e) treating the subject with a non-IO therapy if the subject is determined to have the HLA-I LOH positive status.
208. A method of predicting survival of a subject having cancer, the method comprising:
   (a) receiving a plurality of sequence reads corresponding to a plurality of genomic segments in a sample from a subject, wherein the plurality of sequence reads are aligned with a reference sequence;
   (b) determining:
      (i) whether a number of sequence reads in the plurality of sequence reads exceeds a predetermined sequence read threshold;
      (ii) whether the plurality of genomic segments are germline heterozygous at one or more HLA-I gene loci in the sample; and
      (iii) whether a tumor content of the sample is above a predetermined tumor content threshold;
   (c) when the plurality of genomic segments are germline heterozygous, the number of sequence reads in the plurality of sequence reads exceeds the predetermined sequence read threshold, and the tumor content is above the predetermined tumor content threshold:
      (i) determining a gene minor allele frequency (MAF) of the sample and a segment MAF of the sample; and
      (ii) determining a loss of heterozygosity (LOH) status based on the gene MAF of the sample, the segment MAF of the sample, and the tumor content of the sample, wherein an HLA-I LOH positive status is determined if the tumor content is above the predetermined tumor content threshold and the gene MAF is below a first gene threshold and the segment MAF is below a first segment threshold; and
   (d) wherein:
      (i) if the subject is determined to have the HLA-I LOH positive status, the subject is predicted to have shorter survival when treated with an immune-oncology (IO) therapy, as compared to a subject that was determined to have an HLA-I LOH non-positive status, and
      (ii) if the subject is determined to have an HLA-I LOH non-positive status, the subject is predicted to have longer survival when treated with an IO therapy, as compared to a subject that was determined to have an HLA-I LOH positive status.
209. A method of predicting survival of a subject having cancer, the method comprising:
   (a) receiving a plurality of sequence reads corresponding to a plurality of genomic segments in a sample from a subject, wherein the plurality of sequence reads are aligned with a reference sequence;
   (b) determining:
      (i) whether a number of sequence reads in the plurality of sequence reads exceeds a predetermined sequence read threshold;
      (ii) whether the plurality of genomic segments are germline heterozygous at one or more HLA-I gene loci in the sample; and
      (iii) whether a tumor content of the sample is above a predetermined tumor content threshold;
   (c) when the plurality of genomic segments are germline heterozygous, the number of sequence reads in the plurality of sequence reads exceeds the predetermined sequence read threshold, and the tumor content is above the predetermined tumor content threshold:
      (i) determining a gene minor allele frequency (MAF) of the sample and a segment MAF of the sample; and
      (ii) determining a loss of heterozygosity (LOH) status based on the gene MAF of the sample, the segment MAF of the sample, and the tumor content of the sample, wherein an HLA-I LOH positive status is determined if the tumor content is above the predetermined tumor content threshold and the gene MAF is below a first gene threshold and the segment MAF is below a first segment threshold; and
   (d) wherein if the subject is determined to have the HLA-I LOH positive status, the subject is predicted to have longer survival when treated with a non-immuno-oncology (IO) therapy, as compared to a subject that was treated with an IO therapy.
210. The method of any one of clause 208 to clause 209, further comprising determining a tumor mutational burden (TMB) in the sample from the subject, wherein the predicted survival is further based on the TMB.
211. The method of clause 210, wherein the subject is determined to have a TMB of at least about 4 to 100 mutations/Mb, about 4 to 30 mutations/Mb, 8 to 100 mutations/Mb, 8 to 30 mutations/Mb, 10 to 20 mutations/Mb, less than 4 mutations/Mb, or less than 8 mutations/Mb.
212. The method of clause 211, wherein the TMB is at least about 5 mutations/Mb, is at least about 10 mutations/Mb, at least about 12 mutations/Mb, at least about 16 mutations/Mb, at least about 20 mutations/Mb, or at least about 30 mutations/Mb.
213. The method of clause 211 or clause 212, wherein the TMB is determined based on between about 100 kb to about 10 Mb of genomic sequence.
214. The method of clause 211 or clause 212, wherein the TMB is determined based on between about 0.8 Mb to about 1.1 Mb of genomic sequence.
215. The method of any one of clauses 203-214, further comprising treating the subject determined to have an HLA-I LOH non-positive status with an IO therapy.
216. The method of clause 215, wherein the IO therapy comprises a single IO agent or multiple IO agents.
217. The method of any one of clause 215 or clause 216, wherein the IO therapy comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a cellular therapy, a treatment for cancer being tested in a clinical trial, an immunotherapy, or any combination thereof.
218. The method of any one of clauses 215-217, wherein the IO therapy comprises an immune checkpoint inhibitor.
219. The method of clause 218, wherein the immune checkpoint inhibitor is a PD-1 inhibitor.
220. The method of clause 219, wherein the immune checkpoint inhibitor comprises one or more of nivolumab, pembrolizumab, cemiplimab, or dostarlimab.
221. The method of clause 218, wherein the immune checkpoint inhibitor is a PD-L1-inhibitor.
222. The method of clause 221, wherein the immune checkpoint inhibitor comprises one or more of atezolizumab, avelumab, or durvalumab.
223. The method of clause 218, wherein the immune checkpoint inhibitor is a CTLA-4 inhibitor.
224. The method of clause 223, wherein the CTLA-4 inhibitor comprises ipilimumab.
225. The method of clause 217, wherein the nucleic acid comprises a double-stranded RNA (dsRNA), a small interfering RNA (siRNA), or a small hairpin RNA (shRNA).
226. The method of clause 217, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.
227. The method of any one of clauses 203-226, wherein the non-IO therapy comprises one or more of a small molecule inhibitor, a chemotherapeutic agent, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.
228. The method of clause 227, wherein the chemotherapeutic agent comprises one or more of an alkylating agent, an alkyl sulfonates aziridine, an ethylenimine, a methylamelamine, an acetogenin, a camptothecin, a bryostatin, a callystatin, CC-1065, a cryptophycin, aa dolastatin, a duocarmycin, a eleutherobin, a pancratistatin, a sarcodictyin, a spongistatin, a nitrogen mustard, a nitrosureas, an antibiotic, a dynemicin, a bisphosphonate, an esperamicina a neocarzinostatin chromophore or a related chromoprotein enediyne antiobiotic chromophore, an anti-metabolite, a folic acid analogue, a purine analog, a pyrimidine analog, an androgens, an anti-adrenal, a folic acid replenisher, aldophosphamide glycoside, aminolevulinic acid, eniluracil, amsacrine, bestrabucil, bisantrene, edatraxate, defofamine, demecolcine, diaziquone, elformithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, a PSK polysaccharide complex, razoxane, rhizoxin, sizofiran, spirogermanium, tenuazonic acid, triaziquone, 2,2′,2″-trichlorotriethylamine, a trichothecene, urethan, vindesine, dacarbazine, mannomustine, mitobronitol, mitolactol, pipobroman, gacytosine, arabinoside (“Ara-C”), cyclophosphamide, a taxoid, 6-thioguanine, mercaptopurine, a platinum coordination complex, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluorometlhylomithine (DMFO), a retinoid, capecitabine, carboplatin, procarbazine, plicomycin, gemcitabine, navelbine, farnesyl-protein transferase inhibitors, transplatinum, or any combination thereof.
229. The method of any one of clauses 203-205 or 207-228, further comprising treating the subject determined to have an HLA-I LOH positive status with the non-IO therapy.
230. The method of any one of clauses 203-229, further comprising treating the subject with an additional anti-cancer therapy.
231. The method of clause 230, wherein the additional anti-cancer therapy comprises one of more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.
232. The method of any one of clauses 208-231, wherein the survival is a progression-free survival, an overall survival, a disease-free survival (DFS), an objective response rate (ORR), a time to tumor progression (TTP), a time to treatment failure (TTF), a durable complete response (DCR), or a time to next treatment (TTNT).
233. The method of any one of clauses 208-232, wherein the sample comprises a tissue biopsy sample or a liquid biopsy sample.
234. The method of clause 233, wherein the sample is a tissue biopsy and comprises a tumor biopsy, tumor specimen, or circulating tumor cells.
235. The method of clause 233, wherein the sample is a liquid biopsy sample and comprises blood, serum, plasma, cerebrospinal fluid, sputum, stool, urine, or saliva.
236. The method of any one of clauses 203-235, wherein the sample comprises cells and/or nucleic acids from the cancer.
237. The method of clause 236, wherein the sample comprises mRNA, DNA, circulating tumor DNA (ctDNA), cell-free DNA, cell-free RNA from the cancer, or any combination thereof.
238. The method of clause 237, wherein the sample is a liquid biopsy sample and comprises circulating tumor cells (CTCs).
239. The method of clause 237, wherein the sample is a liquid biopsy sample and comprises cell-free DNA (cfDNA), circulating tumor DNA (ctDNA), or any combination thereof.
240. The method of any one of clauses 203-239, wherein the LOH status of the gene is determined based on sequencing read data derived from sequencing nucleic acid molecules extracted from the sample from the subject.
241. The method of clause 240, wherein the sequencing comprises use of a massively parallel sequencing (MPS) technique, whole genome sequencing (WGS), whole exome sequencing, targeted sequencing, direct sequencing, next-generation sequencing (NGS), or a Sanger sequencing technique.
242. The method of clause 240 or clause 241, wherein the sequencing comprises:
   (a) providing a plurality of nucleic acid molecules obtained from the sample, wherein the plurality of nucleic acid molecules comprises a mixture of tumor nucleic acid molecules and non-tumor nucleic acid molecules;
   (b) optionally, ligating one or more adapters onto one or more nucleic acid molecules from the plurality of nucleic acid molecules;
   (c) amplifying nucleic acid molecules from the plurality of nucleic acid molecules;
   (d) optionally, capturing nucleic acid molecules from the amplified nucleic acid molecules, wherein the captured nucleic acid molecules are captured from the amplified nucleic acid molecules by hybridization to one or more bait molecules; and
   (e) sequencing, by a sequencer, the captured nucleic acid molecules to obtain a plurality of sequence reads corresponding to one or more genomic loci within a subgenomic interval in the sample.
243. The method of clause 242, wherein the adapters comprise one or more of amplification primer sequences, flow cell adapter hybridization sequences, unique molecular identifier sequences, substrate adapter sequences, or sample index sequences.
244. The method of clause 242 or clause 243, wherein amplifying nucleic acid molecules comprises performing a polymerase chain reaction (PCR) technique, a non-PCR amplification technique, or an isothermal amplification technique.
245. The method of any one of clauses 242-244, wherein the one or more bait molecules comprise one or more nucleic acid molecules, each comprising a region that is complementary to a region of a captured nucleic acid molecule.
246. The method of clause 245, wherein the one or more bait molecules each comprise a capture moiety.
247. The method of clause 246, wherein the capture moiety is biotin.
248. The method of any one of clauses 203-247, wherein the cancer is a B cell cancer, a melanoma, breast cancer, lung cancer, bronchus cancer, colorectal cancer or carcinoma, prostate cancer, pancreatic cancer, stomach cancer, ovarian cancer, urinaary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
249. The method of any of clauses 203-248, wherein the subject is a human.
250. The method of any one of clauses 203-249, wherein the subject has previously been treated with an anti-cancer therapy.
251. The method of clause 250, wherein the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

It should be understood from the foregoing that, while particular implementations of the disclosed methods and systems have been illustrated and described, various modifications can be made thereto and are contemplated herein. It is also not intended that the disclosure be limited by the specific examples provided within the specification. While the disclosure has been described with reference to the aforementioned specification, the descriptions and illustrations of the preferable embodiments herein are not meant to be construed in a limiting sense. Furthermore, it shall be understood that all aspects of the disclosure are not limited to the specific depictions, configurations or relative proportions set forth herein which depend upon a variety of conditions and variables. Various modifications in form and detail of the embodiments of the disclosure will be apparent to a person skilled in the art. It is therefore contemplated that the disclosure shall also cover any such modifications, variations and equivalents.

1. A method, comprising:
providing a plurality of nucleic acid molecules obtained from a sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules; sequencing, by a sequencer, the captured nucleic acid molecules to obtain a plurality of sequence reads that represent the captured nucleic acid molecules;
receiving, at one or more processors, sequence read data for the plurality of sequence reads;
identifying, using the one or more processors, a plurality of genomic segments in a sample;
selecting, using the one or more processors, from the plurality of genomic segments, one or more genomic segments that overlap with one or more HLA-I genes in the sample;
determining, using the one or more processors, whether a tumor purity of the sample is in a predetermined range;
when the tumor purity of the sample is in the predetermined range, obtaining, using the one or more processors, a copy number value of the one or more selected genomic segments; and
determining, using the one or more processors, a loss of heterozygosity (LOH) status of the one or more HLA-I genes based on the copy number value.

2. The method of claim 1, further comprising:
performing, using the one or more processors, a quality control procedure prior to obtaining the copy number value; and
foregoing determining the LOH status of the one or more HLA-I genes in accordance with a determination that the sample is associated with a quality control issue.

3. The method of claim 2, wherein the quality control issue includes a sample contamination, a confirmed transplant, a noisy copy number, or a combination thereof.

4. The method of claim 1, wherein the predetermined range comprises a tumor purity between 20-95%.

5. The method of claim 1, wherein the LOH status is determined without baiting the one or more HLA-I genes.

6. The method of claim 1, wherein the one or more HLA-I genes comprise an HLA-A gene, an HLA-B gene, an HLA-C gene, or a combination thereof.

7. The method of claim 1, wherein determining the loss of heterozygosity (LOH) status comprises:
determining the LOH status to be indeterminate if the copy number value is at about a first threshold;
determining the LOH status to be positive if the copy number value is at about a second threshold; and
determining the LOH status to be negative if the copy number value is above a third threshold.

8. The method of claim 7, wherein:
the first threshold is about 0,
the second threshold is about 1, and
the third threshold is about 2.

9. The method of claim 7, further comprising:
when the copy number value is at the third threshold, determining, using the one or more processors, whether the LOH status is a neutral LOH; and
determining, using the one or more processors, the LOH status to be negative if the LOH status does not correspond to the neutral LOH and
determining, using the one or more processors, the LOH status to be positive if the LOH status corresponds to the neutral LOH.

10. The method of claim 1, further comprising determining a zygosity score based on the copy number of the one or more selected genomic segments.

11. The method of claim 10, wherein a zygosity score of about 1 corresponds to a positive LOH status and a zygosity score of about 2 indicates a non-positive LOH status.

12. A method, comprising:
receiving, at one or more processors, one or more sequence reads corresponding to a plurality of genomic segments in a sample from a subject, wherein the one or more sequence reads are aligned with a reference sequence;
determining, using the one or more processors:
whether a number of sequence reads in the one or more sequence reads exceeds a predetermined sequence read threshold;
whether the plurality of genomic segments are germline heterozygous at one or more HLA-I gene loci in the sample; and
whether a tumor content of the sample is above a predetermined tumor content threshold;
when the plurality of genomic segments are germline heterozygous, the number of sequence reads in the one or more sequence reads exceeds the predetermined sequence read threshold, and the tumor content is above the predetermined tumor content threshold:
determining, using the one or more processors, a gene minor allele frequency (MAF) of the sample and a segment MAF of the sample; and
determining, using the one or more processors, a loss of heterozygosity (LOH) status based on the gene MAF of the sample, the segment MAF of the sample, and the tumor content of the sample.

13. The method of claim 12, wherein the sample comprises at least one of a tissue sample, a liquid biopsy sample, or a normal tissue sample.

14. The method of claim 12, wherein the tumor content corresponds to a maximum somatic allele frequency (MSAF), a tumor purity, or a combination thereof.

15. The method of claim 12, wherein the one or more HLA-I gene loci comprise an HLA-A gene, an HLA-B gene, an HLA-C gene, or a combination thereof.

16. The method of claim 12, wherein determining whether the plurality of genomic segments in the sample are germline heterozygous at the one or more HLA-I gene loci in the sample comprises determining, using the one or more processors, an allele fraction of the sample at the one or more HLA-I gene loci is about 0.5.

17. The method of claim 12, wherein determining whether the one or more sequence reads derived from the sample exceeds the predetermined sequence read threshold comprises:
aligning, using the one or more processors, the one or more sequence reads corresponding to the plurality of genomic segments in the sample to the reference sequence, the reference sequence comprising one or more sequences for an HLA allele of the one or more HLA-I gene loci; and
determining, using the one or more processors, the number of sequence reads in the one or more sequence reads aligned with the reference sequence at the one or more HLA-I gene loci; and
comparing, using the one or more processors, the number of sequence reads to the predetermined sequence read threshold.

18. The method of claim 12, wherein the sequence read threshold is one of 1000 reads, 1100 reads, 1200 reads, 1300 reads, 1400 reads, 1500 reads, 1600 reads, 1700 reads, 1800 reads, 1900 reads, 2000 reads, 2100 reads, 2200 reads, 2300 reads, 2400 reads, 2500 reads, 2600 reads, 2700 reads, 2800 reads, 2900 reads, or 3000 reads.

19. The method of claim 12, wherein the tumor content threshold corresponds to a tumor content of 1%, 5%, or 10%.

20. The method of claim 12, further comprising training a model for determining the LOH status of a sample, the training comprising:
receiving, at the one or more processors, one or more reads corresponding to one or more selected genomic segments in a paired sample, wherein the one or more reads corresponding to the one or more selected genomic segments are aligned with the reference sequence;
for each paired sample:
fitting, using the one or more processors, one or more values associated with the one or more reads corresponding to the one or more selected genomic segments to a model;
determining, using the one or more processors, a LOH status of the corresponding paired sample based on the fitted model; and
determining, using the one or more processors, the predetermined sequence read threshold and the tumor content threshold based on the fitted model and the LOH status for the one or more selected genomic segments in the paired sample.
